(19) United States
(12) Patent Application Publication
(10) Pub. No.: US 2022/0257619 A1
(43) Pub. Date: Aug. 18, 2022

(54) LONG-ACTING FORMULATIONS OF TENOFOVIR ALAFENAMIDE

(71) Applicants: Gilead Sciences, Inc., Foster City, CA (US); Durect Corporation, Cupertino, CA (US)

(72) Inventors: Susan Autio, San Jose, CA (US); Keith Edward Branham, Pelham, AL (US); James A. Filice, Los Gatos, CA (US); John W. Gibson, Springville, AL (US); John J. Leonard, Morgan Hill, CA (US); James Matriano, Mountain View, CA (US); Whitney Moro, Birmingham, AL (US); Michael Sekar, Palo Alto, CA (US); Chelsea Alexandra Snyder, San Mateo, CA (US); Robert G. Strickley, San Mateo, CA (US); Raju Subramanian, San Mateo, CA (US); Felix Theeuwes, Los Altos Hills, CA (US); Monica Tijerina, Foster City, CA (US); Jeremy C. Wright, Los Altos, CA (US); Su Il Yum, Los Altos, CA (US); Faye Xu, San Jose, CA (US)

(21) Appl. No.: 17/627,000

(22) PCT Filed: Jul. 17, 2020
(86) PCT No.: PCT/US20/42589

§ 371 Date: Jan. 13, 2022

Related U.S. Application Data
(60) Provisional application No. 62/875,720, filed on Jul. 18, 2019.

(51) Int. Cl. A61K 31/675; A61K 47/26; A61K 47/34; A61K 47/22; A61P 31/18

(52) U.S. Cl. CPC A61K 31/675; A61K 47/26; A61P 31/18; A61K 47/22; A61K 47/34

(57) ABSTRACT

The present disclosure provides long-acting formulations of tenofovir alafenamide, methods of making the same and methods of using the same.

### CROSS REFERENCE TO RELATED APPLICATIONS

This application claims priority to and benefit of U.S. Application No. 62/875,720, filed Jul. 18, 2019, the entire contents of which are hereby incorporated by reference in their entirety.

### JOINT RESEARCH AGREEMENT

The claimed invention was made by or on behalf of one or more of the following parties to a joint research agreement: Durect Corporation and Gilead Sciences, Inc. The agreement was in effect on or before the effective filing date of the claimed invention, and the claimed invention was made as a result of activities undertaken within the scope of the agreement.

### BACKGROUND

Less complicated and less frequent dosing regimens can be advantageous for patients, healthcare providers, and for public health in general. Administration of long-acting medications has several benefits over short-acting oral tablets, e.g., improved convenience and increased compliance, resulting in, e.g., fewer relapses, hospital visits, and healthcare costs.

### SUMMARY

The present disclosure provides long-acting formulations of tenofovir agents, and furthermore demonstrates that provided formulations can achieve particular desirable results (e.g., extended release). Long-acting formulations permit less frequent dosing schedules, which, e.g., can increase patient compliance with antiviral therapy (often comprising multiple drug products administered according to various dosing regimens). In particular, increased patient compliance with antiviral therapy leads to increased efficacy and limits the possibility of developing a resistant viral strain. Therefore, less complicated and less frequent dosing regimens are advantageous.

There remains, however, a need for compositions and methods that provide reproducible, controlled delivery of pharmaceutical active agents with low toxicity. Accordingly, there also remains a need for methods of making these compositions that provide reproducible, controlled delivery of pharmaceutical active agents with low toxicity.

In some embodiments, provided compositions comprise a tenofovir agent and a vehicle comprising a high viscosity liquid carrier material (HVLCM), e.g., sucrose acetate isobutyrate (SAIB). Provided compositions may further comprise a polymer (e.g., poly(lactic acid)(glycolic acid)) and/or a solvent (e.g., propylene carbonate). In some embodiments, provided compositions comprise surprisingly small amounts of water.

In some embodiments, provided compositions comprise tenofovir alafenamide (TAF), or a pharmaceutically acceptable salt thereof, and sucrose acetate isobutyrate. In some embodiments, provided compositions comprise (i) tenofovir alafenamide, or a pharmaceutically acceptable salt thereof; (ii) sucrose acetate isobutyrate; and (iii) propylene carbonate. In some embodiments, provided compositions comprise (i) tenofovir alafenamide, or a pharmaceutically acceptable salt thereof; (ii) sucrose acetate isobutyrate; (iii) propylene carbonate; and (iv) poly(lactic acid)(glycolic acid).

In some embodiments, provided compositions have one or more desirable characteristics, including but not limited to resistance to phase separation, suitable viscosity, stability upon storage, and/or suitable release profile. In some embodiments, provided compositions display a suitable release profile, e.g., a release profile that is sustained at a particular level over a particular period of time and/or that does not display an initial burst release of an active agent.

The present disclosure also provides methods of manufacturing provided compositions, comprising providing a vehicle comprising a HVLCM; and combining the vehicle with a tenofovir agent under suitable conditions to give the provided composition.

The present disclosure also provides methods of administering compositions and dosage forms provided herein. In some embodiments, the present disclosure provides methods of treating and/or preventing HIV and/or HBV infection in a subject in need thereof.

### BRIEF DESCRIPTION OF THE DRAWINGS

FIG. 1 shows an XRPD pattern of tenofovir alafenamide (TAF) sebacate Form I.

FIG. 2 shows a DSC thermogram of TAF sebacate Form I.

FIG. 3 shows the cumulative release (%) of TAF from selected formulations of Table 14. For formulation F5 in FIG. 3, Dulbecco's PBS pH 7.4 buffer was used for the first 10 days. For all other time points in FIG. 3, 20 mM KH₂PO₄, pH 6.0, with 0.9% NaCl buffer was used.

FIG. 4 depicts the delivery rate (μg/h) of TAF from selected formulations of Table 14. For formulations F4, F5, F6, F7 and F10 in FIG. 4, Dulbecco's PBS pH 7.4 buffer was used for the first 10 days. For all other time points in FIG. 4, 20 mM KH₂PO₄, pH 6.0, with 0.9% NaCl buffer was used.

FIG. 5 shows the cumulative release (%) of TAF from selected formulations of Table 14. For formulations F4 and F5 in FIG. 5, Dulbecco's PBS pH 7.4 buffer was used for the first 10 days. For all other time points in FIG. 5, 20 mM KH₂PO₄, pH 6.0, with 0.9% NaCl buffer was used.

FIG. 6 depicts the delivery rate (μg/h) of TAF from selected formulations of Table 14 in 20 mM KH₂PO₄, pH 6.0, with 0.9% NaCl buffer.

FIG. 7 shows the cumulative release (%) of TAF from additional selected formulations of Table 14. For formulations F4 and F5 in FIG. 7, Dulbecco's PBS pH 7.4 buffer was used for the first 10 days. For all other time points in FIG. 7, 20 mM KH₂PO₄, pH 6.0, with 0.9% NaCl buffer was used.

FIG. 8 shows cumulative release (%) of TAF from selected formulations of Table 4B over a 2-day period.

FIG. 9 shows cumulative release (%) of TAF from selected formulations of Table 4B over a 24-day period.

### DETAILED DESCRIPTION

### Definitions

The term “about” or “approximately”, when used herein in reference to a value, refers to a value that is similar, in context to the referenced value. In general, those skilled in the art, familiar with the context, will appreciate the relevant degree of variance encompassed by “about” in that context.

As used herein, the term “administering” or “administration” typically refers to the administration of a composition to a subject to achieve delivery of an agent that is, or is included, in a composition to a target site or a site to be treated. Those of ordinary skill in the art will be aware of a variety of routes that may, in appropriate circumstances, be utilized for administration to a subject, for example a human. For example, in some embodiments, administration may be parenteral. In some embodiments, administration may be by injection (e.g., intramuscular, intravenous, or subcutaneous injection). In some embodiments, administration may involve only a single dose. In some embodiments, administration may involve application of a fixed number of doses. In some embodiments, administration may involve dosing that is intermittent (e.g., a plurality of doses separated in time) and/or periodic (e.g., individual doses separated by a common period of time). In some embodiments, administration may involve continuous dosing (e.g., perfusion) for at least a selected period of time.

As used herein, the term “combination therapy” refers to those situations in which a subject is simultaneously exposed to two or more therapeutic or prophylactic regimens (e.g., two or more therapeutic or prophylactic agents). In some embodiments, the two or more regimens may be administered simultaneously; in some embodiments, such regimens may be administered sequentially (e.g., all “doses” of a first regimen are administered prior to administration of any doses of a second regimen); in some embodiments, such agents are administered in overlapping dosing regimens. In some embodiments, “administration” of combination therapy may involve administration of one or more agent(s) or modality(ies) to a subject receiving the other agent(s) or modality(ies) in the combination. For clarity, combination therapy does not require that individual agents be administered together in a single composition (or even necessarily at the same time), although in some embodiments, two or more agents, or active moieties thereof, may be administered together in a combination composition, or even in a combination compound (e.g., as part of a single chemical complex or covalent entity).

As used herein, the term “comparable” refers to two or more agents, entities, situations, sets of conditions, etc., that may not be identical to one another but that are sufficiently similar to permit comparison there between so that one skilled in the art will appreciate that conclusions may reasonably be drawn based on differences or similarities observed. In some embodiments, comparable sets of conditions, circumstances, individuals, or populations are characterized by a plurality of substantially identical features and one or a small number of varied features. Those of ordinary skill in the art will understand, in context, what degree of identity is required in any given circumstance for two or more such agents, entities, situations, sets of conditions, etc., to be considered comparable. For example, those of ordinary skill in the art will appreciate that sets of circumstances, individuals, or populations are comparable to one another when characterized by a sufficient number and type of substantially identical features to warrant a reasonable conclusion that differences in results obtained or phenomena observed under or with different sets of circumstances, individuals, or populations are caused by or indicative of the variation in those features that are varied.

As used herein, the term “dosage form” refers to a physically discrete unit of an active agent (e.g., a therapeutic, prophylactic, or diagnostic agent) for administration to a subject. Typically, each such unit contains a predetermined quantity of active agent. In some embodiments, such quantity is a unit dosage amount (or a whole fraction thereof) appropriate for administration in accordance with a dosing regimen that has been determined to correlate with a desired or beneficial outcome when administered to a relevant population (i.e., with a prophylactic or therapeutic dosing regimen). Those of ordinary skill in the art appreciate that the total amount of a composition or agent administered to a particular subject is determined by one or more attending physicians and may involve administration of multiple dosage forms.

The term “pharmaceutically acceptable salt”, as used herein, refers to salts of such compounds that are appropriate for use in pharmaceutical contexts, i.e., salts which are, within the scope of sound medical judgment, suitable for use in contact with the tissues of humans and/or animals without undue toxicity, irritation, allergic response and the like, and are commensurate with a reasonable benefit/risk ratio. Pharmaceutically acceptable salts are well known in the art. For example, S. M. Berge, et al. describes several pharmaceutically acceptable salts in detail in J. Pharmaceutical Sciences, 66: 1-19 (1977), which is hereby incorporated by reference in its entirety.

As used herein, the term “subject” refers to an organism, typically a mammal (e.g., a human). In some embodiments, a subject is suffering from a relevant disease, disorder or condition. In some embodiments, a subject is healthy. In some embodiments, a human subject is an adult, adolescent, or pediatric subject. In some embodiments, a subject is susceptible to a disease, disorder, or condition. In some embodiments, a subject displays one or more symptoms or characteristics of a disease, disorder or condition. In some embodiments, a subject does not display any symptom or characteristic of a disease, disorder, or condition. In some embodiments, a subject is someone with one or more features characteristic of susceptibility to or risk of a disease, disorder, or condition. In some embodiments, a subject is a patient. In some embodiments, a subject is an individual to whom diagnosis and/or therapy and/or prophylaxis is and/or has been administered.

As used herein, the term “tenofovir agent” refers to a compound or entity that, when administered to a subject, delivers to that subject a tenofovir active moiety. In some embodiments, a tenofovir agent is or comprises tenofovir. In some embodiments, a tenofovir agent is or comprises tenofovir alafenamide. In some embodiments, a tenofovir agent is or comprises tenofovir diisoproxil fumarate. In some embodiments, a tenofovir agent is provided and/or utilized in a salt form (e.g., as a sebacate salt). In some embodiments, a tenofovir agent is a prodrug of tenofovir wherein tenofovir or tenofovir diphosphate is the intended metabolite for its therapeutic, prophylactic, or diagnostic effect. In some embodiments, a tenofovir agent is provided and/or utilized as a salt, co-crystal, free acid or base, solvate, ester, hydrate, polymorph, or anhydrous form.

As used herein, “therapeutically effective amount” is an amount that produces the desired effect for which it is administered. In some embodiments, the term “therapeutically effective amount” or “therapeutically effective dose” means an amount that is sufficient, when administered to a population suffering from or susceptible to a disease, disorder, and/or condition in accordance with a therapeutic dosing regimen, to treat or prevent the disease, disorder, and/or condition. In some embodiments, a therapeutically effective amount is one that reduces the incidence and/or severity of, stabilizes one or more characteristics of, and/or delays onset of, one or more symptoms of the disease, disorder, and/or condition. Those of ordinary skill in the art will appreciate that the term “therapeutically effective amount” does not in fact require successful treatment or prevention be achieved in a particular individual. Rather, a therapeutically effective amount may be that amount that provides a particular desired pharmacological response in a significant number of subjects when administered to patients in need of such treatment or prevention. In some embodiments, reference to a therapeutically effective amount may be a reference to an amount as measured in one or more specific tissues (e.g., a tissue affected by the disease, disorder or condition) or fluids (e.g., blood, saliva, serum, sweat, tears, urine, etc.). Those of ordinary skill in the art will appreciate that, in some embodiments, a therapeutically effective amount may be formulated and/or administered in a single dose. In some embodiments, a therapeutically effective amount may be formulated and/or administered in a plurality of doses, for example, as part of a dosing regimen.

### Provided Compositions:

Provided herein are novel compositions that comprise and/or deliver a tenofovir agent and are formulated for controlled release (i.e., a long-acting formulation). Provided compositions are useful in methods described herein.

### Components of Provided Compositions

### Tenofovir Agent:

Provided compositions comprise a tenofovir agent. In some embodiments, the tenofovir agent is or comprises tenofovir alafenamide. Tenofovir alafenamide is a nucleotide reverse transcriptase inhibitor having the following structure:

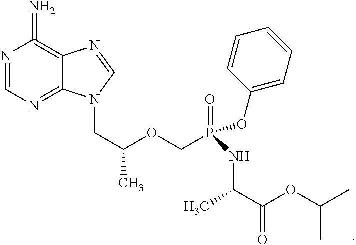

Tenofovir alafenamide was first described in WO 02/08241, the entire contents of which are hereby incorporated by reference. Its IUPAC name is (S)-isopropyl-2-(((S)-((((R)-1-(6-amino-9H-purin-9-yl)propan-2-yl)oxy)methyl)(phenoxy)phosphoryl)amino)propanoate. It is also referred to as {9-[(R)-2-[[(S)-[[(S)-1-(isopropoxycarbonyl)ethyl]amino]phenoxyphosphinyl]-methoxy]propyl]adenine}.

In some embodiments, tenofovir alafenamide is provided and/or utilized as a pharmaceutically acceptable salt. In some embodiments, tenofovir alafenamide, or a pharmaceutically acceptable salt thereof, is provided and/or utilized as a solid form (e.g., an amorphous solid form, a crystalline solid form, or a mixture thereof). For example, salt forms of tenofovir alafenamide and solid forms thereof are described in WO 2013/025788, WO 2016/205141, and WO 2018/144390, each of which is hereby incorporated by reference in its entirety.

In some embodiments, tenofovir alafenamide is provided and/or utilized as a pharmaceutically acceptable salt form selected from tenofovir alafenamide hemipamoate, tenofovir alafenamide sebacate, tenofovir alafenamide napsylate, tenofovir alafenamide orotate, tenofovir alafenamide vanillate, and tenofovir alafenamide bis-xinafoate.

In some embodiments, provided compositions comprise tenofovir alafenamide sebacate. In some embodiments, tenofovir alafenamide sebacate is provided and/or utilized in an amorphous form, a crystalline form, or a mixture thereof.

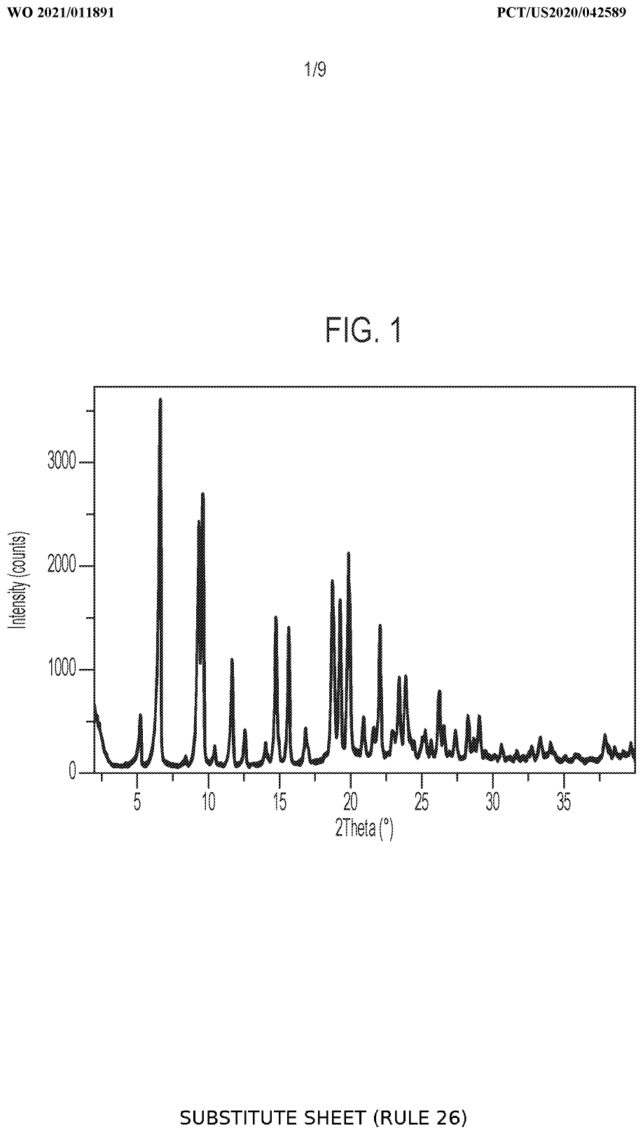

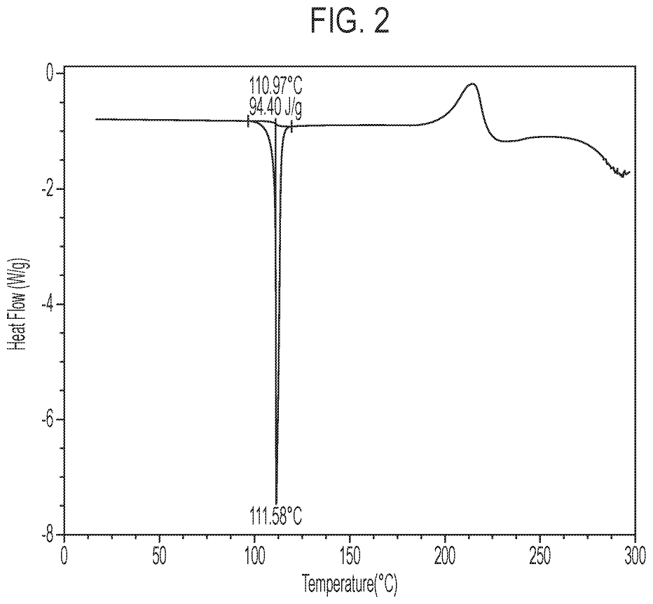

In some embodiments, tenofovir alafenamide sebacate is provided and/or utilized in a crystalline form. In some embodiments, a crystalline form of tenofovir alafenamide sebacate is Form I, wherein Form I is characterized by having an X-ray powder diffraction (XRPD) pattern that is substantially as shown in FIG. 1. In some embodiments, a crystalline form of tenofovir alafenamide sebacate is Form I, wherein Form I is characterized by having a differential scanning calorimetry (DSC) thermogram substantially as shown in FIG. 2. In some embodiments, crystalline tenofovir alafenamide sebacate Form I has an X-ray powder diffraction (XRPD) pattern that is substantially as shown in FIG. 1 and a differential scanning calorimetry (DSC) thermogram substantially as shown in FIG. 2.

Those skilled in the art will appreciate that presence of a particular crystalline form of a tenofovir agent can be determined by detecting characteristic element(s) (e.g., sets of peaks) of an analytic assessment such as an XRPD pattern or DSC thermogram.

In some embodiments, crystalline tenofovir alafenamide sebacate Form I has an XRPD pattern displaying at least two, at least three, at least four, at least five, at least six, at least seven, or at least eight of the degree 2θ-reflections with the greatest intensity as the XRPD pattern substantially as shown in FIG. 1.

In some embodiments, crystalline tenofovir alafenamide sebacate Form I has an XRPD pattern comprising degree 2θ-reflections (+/−0.2 degrees 2θ) at one or more of 5.3°, 6.6°, 9.4°, 9.6°, and 19.8°. In some embodiments, crystalline tenofovir alafenamide sebacate Form I has an XRPD pattern comprising degree 2θ-reflections (+/−0.2 degrees 2θ) at one or more of 5.3°, 6.6°, 9.4°, 9.6°, and 19.8° and one or more of the degree 2θ-reflections (+/−0.2 degrees 2θ) at 14.8°, 15.7°, 18.7°, 19.3° and 22.1°. In some embodiments, crystalline tenofovir alafenamide sebacate Form I has an XRPD pattern comprising degree 2θ-reflections (+/−0.2 degrees 2θ) at one or more of 5.3°, 6.6°, 9.4°, 9.6°, and 19.8° and one of the degree 2θ-reflections (+/−0.2 degrees 2θ) at 14.8°, 15.7°, 18.7°, 19.3° and 22.1°. In some embodiments, crystalline tenofovir alafenamide sebacate Form I has an XRPD pattern comprising degree 2θ-reflections (+/−0.2 degrees 2θ) at one or more of 5.3°, 6.6°, 9.4°, 9.6°, and 19.8° and two of the degree 2θ-reflections (+1-0.2 degrees 2θ) at 14.8°, 15.7°, 18.7°, 19.3° and 22.1°. In some embodiments, crystalline tenofovir alafenamide sebacate Form I has an XRPD pattern comprising degree 2θ-reflections (+1-0.2 degrees 2θ) at one or more of 5.3°, 6.6°, 9.4°, 9.6°, and 19.8° and three of the degree 2θ-reflections (+1-0.2 degrees 2θ) at 14.8°, 15.7°, 18.7°, 19.3° and 22.1°. In some embodiments, crystalline tenofovir alafenamide sebacate Form I has an XRPD pattern comprising degree 2θ-reflections (+1-0.2 degrees 2θ) at one or more of 5.3°, 6.6°, 9.4°, 9.6°, and 19.8° and four of the degree 2θ-reflections (+1-0.2 degrees 2θ) at 14.8°, 15.7°, 18.7°, 19.3° and 22.1°. In some embodiments, crystalline tenofovir alafenamide sebacate Form I has an XRPD pattern comprising degree 2θ-reflections (+1-0.2 degrees 2θ) at one or more of 5.3°, 6.6°, 9.4°, 9.6°, 14.8°, 15.7°, 18.7°, 19.3°, 19.8°, and 22.1°.

In some embodiments, crystalline tenofovir alafenamide sebacate Form I has an XRPD pattern comprising degree 2θ-reflections (+1-0.2 degrees 2θ) at one or more of 5.3°, 6.6°, 9.4°, 9.6°, 14.8°, 15.7°, 18.7°, 19.3°, 19.8°, and 22.1° and one or more of the degree 2θ-reflections (+1-0.2 degrees 2θ) at 11.7°, 12.6°, 20.9°, 23.4°, 23.8°, 26.2°, 28.2°, and 29.0°. In some embodiments, crystalline tenofovir alafenamide sebacate Form I has an XRPD pattern comprising degree 2θ-reflections (+1-0.2 degrees 2θ) at one or more of 5.3°, 6.6°, 9.4°, 9.6°, 14.8°, 15.7°, 18.7°, 19.3°, 19.8°, and 22.1° and one of the degree 2θ-reflections (+1-0.2 degrees 2θ) at 11.7°, 12.6°, 20.9°, 23.4°, 23.8°, 26.2°, 28.2°, and 29.0°. In some embodiments, crystalline tenofovir alafenamide sebacate Form I has an XRPD pattern comprising degree 2θ-reflections (+1-0.2 degrees 2θ) at one or more of 5.3°, 6.6°, 9.4°, 9.6°, 14.8°, 15.7°, 18.7°, 19.3°, 19.8°, and 22.1° and two of the degree 2θ-reflections (+1-0.2 degrees 2θ) at 11.7°, 12.6°, 20.9°, 23.4°, 23.8°, 26.2°, 28.2°, and 29.0°. In some embodiments, crystalline tenofovir alafenamide sebacate Form I has an XRPD pattern comprising degree 2θ-reflections (+1-0.2 degrees 2θ) at one or more of 5.3°, 6.6°, 9.4°, 9.6°, 14.8°, 15.7°, 18.7°, 19.3°, 19.8°, and 22.1° and three of the degree 20-reflections (+1-0.2 degrees 2θ) at 11.7°, 12.6°, 20.9°, 23.4°, 23.8°, 26.2°, 28.2°, and 29.0°. In some embodiments, crystalline tenofovir alafenamide sebacate Form I has an XRPD pattern comprising degree 2θ-reflections (+1-0.2 degrees 2θ) at one or more of 5.3°, 6.6°, 9.4°, 9.6°, 14.8°, 15.7°, 18.7°, 19.3°, 19.8°, and 22.1° and four of the degree 2θ-reflections (+1-0.2 degrees 2θ) at 11.7°, 12.6°, 20.9°, 23.4°, 23.8°, 26.2°, 28.2°, and 29.0°. In some embodiments, crystalline tenofovir alafenamide sebacate Form I has an XRPD pattern comprising degree 2θ-reflections (+1-0.2 degrees 2θ) at one or more of 5.3°, 6.6°, 9.4°, 9.6°, 14.8°, 15.7°, 18.7°, 19.3°, 19.8°, and 22.1° and five of the degree 2θ-reflections (+1-0.2 degrees 2θ) at 11.7°, 12.6°, 20.9°, 23.4°, 23.8°, 26.2°, 28.2°, and 29.0°. In some embodiments, crystalline tenofovir alafenamide sebacate Form I has an XRPD pattern comprising degree 2θ-reflections (+1-0.2 degrees 2θ) at one or more of 5.3°, 6.6°, 9.4°, 9.6°, 14.8°, 15.7°, 18.7°, 19.3°, 19.8°, and 22.1° and six of the degree 20-reflections (+1-0.2 degrees 2θ) at 11.7°, 12.6°, 20.9°, 23.4°, 23.8°, 26.2°, 28.2°, and 29.0°. In some embodiments, crystalline tenofovir alafenamide sebacate Form I has an XRPD pattern comprising degree 2θ-reflections (+1-0.2 degrees 2θ) at one or more of 5.3°, 6.6°, 9.4°, 9.6°, 14.8°, 15.7°, 18.7°, 19.3°, 19.8°, and 22.1° and seven of the degree 2θ-reflections (+1-0.2 degrees 2θ) at 11.7°, 12.6°, 20.9°, 23.4°, 23.8°, 26.2°, 28.2°, and 29.0°. In some embodiments, crystalline tenofovir alafenamide sebacate Form I has an XRPD pattern comprising degree 2θ-reflections (+1-0.2 degrees 2θ) at one or more of 5.3°, 6.6°, 9.4°, 9.6°, 11.7°, 12.6°, 14.8°, 15.7°, 18.7°, 19.3°, 19.8°, 20.9°, 22.1°, 23.4°, 23.8°, 26.2°, 28.2°, and 29.0°. In some embodiments, crystalline tenofovir alafenamide sebacate Form I has an XRPD pattern comprising any five degree 2θ-reflections (+1-0.2 degrees 2θ) selected from the group consisting of 5.3°, 6.6°, 9.4°, 9.6°, 10.5°, 11.7°, 12.6°, 14.0°, 14.8°, 15.7°, 16.9°, 18.7°, 19.3°, 19.8°, 20.9°, 21.6°, 22.1°, 22.9°, 23.4°, 23.8°, 25.3°, 26.2°, 26.5°, 27.4°, 28.2°, 28.7°, 29.0°, 33.3°, and 37.9°. In some embodiments, crystalline tenofovir alafenamide sebacate Form I has an XRPD pattern comprising any seven degree 2θ-reflections (+1-0.2 degrees 2θ) selected from the group consisting of 5.3°, 6.6°, 9.4°, 9.6°, 10.5°, 11.7°, 12.6°, 14.0°, 14.8°, 15.7°, 16.9°, 18.7°, 19.3°, 19.8°, 20.9°, 21.6°, 22.1°, 22.9°, 23.4°, 23.8°, 25.3°, 26.2°, 26.5°, 27.4°, 28.2°, 28.7°, 29.0°, 33.3°, and 37.9°. In some embodiments, crystalline tenofovir alafenamide sebacate Form I has an XRPD pattern comprising any ten degree 2θ-reflections (+1-0.2 degrees 2θ) selected from the group consisting of 5.3°, 6.6°, 9.4°, 9.6°, 10.5°, 11.7°, 12.6°, 14.0°, 14.8°, 15.7°, 16.9°, 18.7°, 19.3°, 19.8°, 20.9°, 21.6°, 22.1°, 22.9°, 23.4°, 23.8°, 25.3°, 26.2°, 26.5°, 27.4°, 28.2°, 28.7°, 29.0°, 33.3°, and 37.9°.

In some embodiments, provided compositions do not comprise any active agent other than a tenofovir agent.

In some embodiments, provided compositions comprise about 1 wt %, about 2 wt %, about 5 wt %, about 8 wt %, about 10 wt %, about 12 wt %, about 15 wt %, about 18 wt %, about 20 wt %, about 25 wt %, about 30 wt %, about 40 wt %, or about 50 wt % tenofovir agent, based on the weight of the vehicle or the total weight of the composition. In some embodiments, provided compositions comprise from about 1 wt % to about 50 wt %, about 2 wt % to about 40 wt %, about 5 wt % to about 30 wt %, about 10 wt % to about 25 wt %, about 10 wt % to about 20 wt %, about 5 wt % to about 10 wt %, about 5 wt % to about 15 wt %, about 8 wt % to about 18 wt %, about 5 wt % to about 20 wt %, about 10 wt % to about 15 wt %, or about 10 wt % to about 12 wt % tenofovir agent, based on the weight of the vehicle or the total weight of the composition.

In some embodiments, provided compositions comprise about 1 wt %, about 2 wt %, about 5 wt %, about 8 wt %, about 10 wt %, about 12 wt %, about 15 wt %, about 18 wt %, about 20 wt %, about 25 wt %, about 30 wt %, about 40 wt %, or about 50 wt % tenofovir alafenamide, based on the weight of the vehicle or the total weight of the composition. In some such embodiments, the tenofovir alafenamide is tenofovir alafenamide sebacate. In some embodiments, provided compositions comprise from about 1 wt % to about 50 wt %, about 2 wt % to about 40 wt %, about 5 wt % to about 30 wt %, about 10 wt % to about 25 wt %, about 10 wt % to about 20 wt %, about 5 wt % to about 10 wt %, about 5 wt % to about 15 wt %, about 8 wt % to about 18 wt %, about 5 wt % to about 20 wt %, about 10 wt % to about 15 wt %, or about 10 wt % to about 12 wt % tenofovir alafenamide, based on the weight of the vehicle or the total weight of the composition. In some such embodiments, the tenofovir alafenamide is tenofovir alafenamide sebacate.

It will be understood that compositions or formulations comprising tenofovir alafenamide may comprise tenofovir alafenamide in one of several forms (e.g., free base form, salt form, etc.). It will be understood, therefore, that reference to an amount (e.g., in mg or wt %) of tenofovir alafenamide means the amount of tenofovir alafenamide in free base form. Accordingly, tenofovir alafenamide may be provided and/or utilized as, e.g., a salt form of tenofovir alafenamide such that the amount of the salt (or other form) is an amount that corresponds to the “free base equivalent” of tenofovir alafenamide. For example, “25 mg tenofovir alafenamide” means, e.g., approx. 35.6 mg of tenofovir alafenamide sebacate, approx. 35.2 mg tenofovir alafenamide hemipamoate, etc.

Without wishing to be bound by any particular theory, salt forms of tenofovir alafenamide that are poorly soluble may be particularly useful in provided compositions. Furthermore, the present disclosure encompasses the recognition that compositions comprising both a long-acting salt of tenofovir alafenamide (e.g., a poorly soluble salt) and a HVLCM, as described herein, are particularly effective as long-acting formulations. Accordingly, in some embodiments, provided compositions comprise tenofovir alafenamide as a pharmaceutically acceptable salt, wherein the tenofovir alafenamide salt has a solubility of less than about 5 mg/mL, less than about 2 mg/mL, or less than about 1 mg/mL in deionized water at about 22° C. In some embodiments, provided compositions comprise tenofovir alafenamide as a pharmaceutically acceptable salt, wherein the tenofovir alafenamide salt has a solubility of less than about 10 mg/mL, less than about 5 mg/mL, less than about 2 mg/mL, or less than about 1 mg/mL in the composition at about 25° C. In some embodiments, provided compositions comprise tenofovir alafenamide as a pharmaceutically acceptable salt, wherein the tenofovir alafenamide salt has a solubility of about 0.2 mg/mL to about 10 mg/mL, about 0.5 mg/mL to about 8 mg/mL, about 1 mg/mL to about 6 mg/mL, or about 2 mg/mL to about 5 mg/mL in the composition at about 25° C.

In some embodiments, the tenofovir agent is dissolved or suspended in the composition. Particles comprising the tenofovir agent, which are used to make the provided compositions, typically have a median particle size, as measured by laser diffraction, from about 0.1 μm to about 100 μm, from about 0.2 μm to about 90 μm, from about 0.25 μm to about 80 μm, from about 0.5 μm to about 70 μm, from about 1 μm to about 70 μm, from about 2 μm to about 60 μm, from about 5 μm to about 60 μm, from about 10 μm to about 50 μm, or from about 10 μm to about 40 μm.

In the context of the present disclosure, the median particle size, as measured by laser diffraction, refers to the size of the particles before addition with the vehicle. Thus, the recited compositions are “made from” or “obtainable by combining” the particles comprising the tenofovir agent and the one or more further specified components.

### Vehicle:

In some embodiments, the present disclosure provides compositions comprising a tenofovir agent and a vehicle. In some embodiments, provided compositions comprise about 50 wt %, about 55 wt %, about 60 wt %, about 65 wt %, about 70 wt %, about 75 wt %, about 80 wt %, about 85 wt %, about 90 wt %, or about 95 wt % vehicle, based on the total weight of the composition. In some embodiments, provided compositions comprise from about 50 wt % to about 99 wt %, about 60 wt % to about 98 wt %, about 70 wt % to about 95 wt %, about 75 wt % to about 90 wt %, or about 80 wt % to about 90 wt % vehicle, based on the total weight of the composition.

In some embodiments, the vehicle comprises one or more of a high viscosity liquid carrier material (HVLCM), a polymer (e.g., a lactic acid-based polymer), and a solvent, or any combination thereof. In some embodiments, the vehicle comprises a HVLCM. In some embodiments, the vehicle comprises a polymer (e.g., a lactic acid-based polymer). In some embodiments, the vehicle comprises a solvent. In some embodiments, the vehicle comprises a HVLCM, a polymer (e.g., a lactic acid-based polymer), and a solvent.

In some embodiments, the vehicle comprises a HVLCM, a polymer, and a solvent, wherein the relative amounts, expressed as weight ratios, are about 1:0.1-2:0.3-10, 1:0.2-1:0.4-5, 1:0.3-0.5:0.5-1, or 1:0.1-0.5:0.3-0.9, respectively.

### High Viscosity Liquid Carrier Materials (HVLCM)

In some embodiments, the present disclosure provides compositions comprising a tenofovir agent, and further comprising one or more high viscosity liquid carrier materials (HVLCMs). In some embodiments, provided compositions comprise a tenofovir agent and a vehicle comprising one or more HVLCMs. Typically, a HVLCM suitable for use in provided compositions is non-polymeric and/or not water-soluble. As used herein, the term “not water-soluble” or “non-water soluble” refers to a material that is soluble in water to a degree of less than 1% by weight under ambient conditions.

In some embodiments, the HVLCM has a viscosity of at least 5000 cP at 37° C. and does not crystallize when neat at 25° C. and at 1 atmosphere. For example, the HVLCM may have a viscosity of at least 10,000 cP, at least 15,000 cP, at least 20,000 cP, at least 25,000 cP, at least 50,000 cP, at least 100,000 cP, at least 200,000 cP, or at least 300,000 cP at 37° C.

In some embodiments, the one or more HVLCMs are selected from sucrose acetate isobutyrate, stearate esters (such as stearate esters of propylene glycol, glyceryl, diethylaminoethyl, and glycol), stearate amides or other long-chain fatty acid amides (such as N,N′-ethylene distearamide, stearamide monoethanolamine, stearamide diethanolamine, or ethylene bistearamide), cocoamine oxide, long-chain fatty alcohols (such as cetyl alcohol and stearyl alcohol), long-chain esters (such as myristyl myristate and beheny erucate), glyceryl phosphates, and acetylated sucrose distearate (i.e., Crodesta A-10). Additional materials suitable for use as the HVLCM are described in US 2004/0101557, the entire contents of which are hereby incorporated by reference.

Without wishing to be bound by any particular theory, the amount of HVLCM in provided compositions can depend on the desired properties of the composition and/or on the solvent capacity of a solvent also present in the composition. For example, if the solvent has poor solvent capacity, then the amount of solvent may be large and a corresponding reduction in the amount of HVLCM is necessary.

In some embodiments, provided compositions comprise about 5 wt %, about 10 wt %, about 25 wt %, about 30 wt %, about 40 wt %, about 50 wt %, about 55 wt %, about 60 wt %, about 70 wt %, about 80 wt %, or about 90 wt % HVLCM, based on the weight of the vehicle or the total weight of the composition. In some embodiments, provided compositions comprise from about 5 wt % to about 95 wt %, about 5 wt % to about 90 wt %, about 10 wt % to about 90 wt %, about 25 wt % to about 80 wt %, about 30 wt % to about 70 wt %, or about 40 wt % to about 60 wt % HVLCM, based on the weight of the vehicle or the total weight of the composition. In some embodiments, provided compositions comprise from about 5 wt % to about 95 wt %, about 5 wt % to about 90 wt %, about 10 wt % to about 90 wt %, about 25 wt % to about 80 wt %, about 25 wt % to about 65 wt %, about 30 wt % to about 70 wt %, or about 40 wt % to about 60 wt % HVLCM, based on the weight of the vehicle or the total weight of the composition.

In some embodiments, the HVLCM is sucrose acetate isobutyrate (SAIB). SAIB comprises a sucrose molecule esterified with acetic acid and isobutyric acid.

SAIB is orally non-toxic and is currently used to stabilize emulsions in the food industry. It is a very viscous liquid yet undergoes dramatic changes in viscosity in the presence of heat and/or the addition of small quantities of solvent(s). For example, SAIB has a viscosity of about 2 million cP at about 25° C., of about 320,000 cP at 37° C., and of about 600 cP at 80° C. (US 2009/0087408 and U.S. Pat. No. 8,133,507, each of which is hereby incorporated by reference in its entirety). SAM is soluble in a large number of biocompatible solvents. When in solution or in an emulsion, SAM can be administered via injection or an aerosol spray. SAIB is compatible with cellulose esters and other polymers suitable for use in provided compositions.

In some embodiments, provided compositions comprise about 5 wt %, about 10 wt %, about 25 wt %, about 30 wt %, about 40 wt %, about 50 wt %, about 55 wt %, about 60 wt %, about 70 wt %, about 80 wt %, or about 90 wt % SAIB, based on the weight of the vehicle or the total weight of the composition. In some embodiments, provided compositions comprise from about 5 wt % to about 95 wt %, about 5 wt % to about 90 wt %, about 10 wt % to about 90 wt %, about 25 wt % to about 80 wt %, about 30 wt % to about 70 wt %, or about 40 wt % to about 60 wt % SAIB, based on the weight of the vehicle or the total weight of the composition. In some embodiments, provided compositions comprise from about 5 wt % to about 95 wt %, about 5 wt % to about 90 wt %, about 10 wt % to about 90 wt %, about 25 wt % to about 80 wt %, about 25 wt % to about 65 wt %, about 30 wt % to about 70 wt %, or about 40 wt % to about 60 wt % SAIB, based on the weight of the vehicle or the total weight of the composition.

### Polymers

In some embodiments, the present disclosure provides compositions comprising a tenofovir agent, and further comprising one or more polymers. In some embodiments, provided compositions further comprise a vehicle comprising one or more polymers. In some embodiments, provided compositions comprise a tenofovir agent and a vehicle comprising one or more polymers. In some embodiments, the polymer is a lactic acid-based polymer, a glycolic acid-based polymer, an orthoester-based polymer, and/or a trimethylene carbonate-based polymer. In some embodiments, the polymer is a lactic acid-based polymer. Polymers that are particularly useful in provided compositions are biodegradable and/or biocompatible.

Without wishing to be bound by any particular theory, particularly useful polymers may alter the release profile of the tenofovir agent, add integrity to the composition and/or otherwise modify the properties of the composition. For example, it is desirable for provided compositions to comprise a HVLCM and a polymer which are miscible, in order to avoid phase separation of the HVLCM and the polymer. Phase separation of the HVLCM and the polymer is undesirable, because remixing may be difficult, e.g., at the time of administration, and improper mixing can affect the release profile of the tenofovir agent. Accordingly, in some embodiments, the polymer is sufficiently miscible with the HVLCM. In some embodiments, the polymer is sufficiently soluble in the composition.

In some embodiments, the polymer is or comprises a linear polymer. In some embodiments, the polymer is or comprises a branched polymer.

In some embodiments, the polymer is or comprises a saturated polymer. In some embodiments, the polymer is or comprises an unsaturated polymer.

In some embodiments, the polymer is or comprises a homopolymer. In some embodiments, the polymer is or comprises poly(lactic acid), i.e., polylactide. The terms “poly(lactic acid)” and polylactide are used interchangeably herein.

In some embodiments, the polymer is or comprises a copolymer. In some embodiments, the polymer (e.g., a lactic acid-based polymer) is or comprises a copolymer of lactic acid repeat units and another suitable repeat unit. Suitable repeat units include, but are not limited to, glycolic acid repeat units, glycolide repeat units, polyethylene glycol repeat units, caprolactone repeat units, valerolactone repeat units, trimethylene-carbonate repeat units, and the like. As used herein, “repeat unit” refers to a repetitive structural unit of a polymer. In some embodiments herein, repeat units are depicted within a set of square brackets as depicted below. It will be appreciated that each repeat unit is independent of the other, e.g., if two different monomers are used in a polymerization reaction.

In some embodiments, the polymer (e.g., a lactic acid-based polymer) is or comprises a copolymer of lactic acid repeat units and glycolic acid repeat units. Accordingly, in some embodiments, the lactic acid-based polymer is or comprises poly(lactic acid)(glycolic acid) (PLGA), i.e., poly(lactide)(glycolide). The terms “poly(lactic acid)(glycolic acid)” and “poly(lactide)(glycolide)” are used interchangeably herein.

In some embodiments, the PLGA comprises lactic acid repeat units and glycolic acid repeat units in a molar ratio of about 100:0, about 90:10, about 85:15, about 75:25, about 65:35, or about 50:50. In some embodiments, the PLGA comprises lactic acid repeat units and glycolic acid repeat units in a molar ratio of about 100:0, about 95:5, about 90:10, about 85:15, about 75:25, about 65:35, or about 50:50. In some embodiments, the PLGA comprises lactic acid repeat units and glycolic acid repeat units in a molar ratio of from about 100:0 to about 50:50, from about 100:0 to about 70:30, from about 100:0 to about 75:25, or from about 95:5 to about 85:15. In some embodiments, the PLGA comprises lactic acid repeat units and glycolic acid repeat units in a molar ratio of from about 100:0 to about 50:50, from about 100:0 to about 70:30, from about 100:0 to about 75:25, from about 95:5 to about 65:35, or from about 95:5 to about 85:15.

Without wishing to be bound by any particular theory, PLGA with a higher molar ratio of lactic acid repeat units to glycolic acid repeat units tend to be more suitable for use with SAIB and/or tend to provide longer release profiles. Accordingly, in some embodiments, the PLGA comprises lactic acid repeat units and glycolic acid repeat units in a molar ratio of greater than about 70:30, greater than about 75:25, greater than about 85:15, or greater than about 90:10.

In some embodiments, the polymer (e.g., PLGA) has a weight average molecular weight of about 4 kDa, about 8 kDa, about 10 kDa, about 12 kDa, about 14 kDa, about 16 kDa, about 18 kDa, about 20 kDa, about 30 kDa, about 40 kDa, or about 50 kDa. In some embodiments, the polymer (e.g., PLGA) has a weight average molecular weight of about 4 kDa, about 8 kDa, about 10 kDa, about 12 kDa, about 14 kDa, about 16 kDa, about 18 kDa, about 20 kDa, about 30 kDa, about 40 kDa, about 50 kDa, about 60 kDa, or about 70 kDa. In some embodiments, the polymer (e.g., PLGA) has a weight average molecular weight of from about 1 kDa to about 50 kDa, from about 4 kDa to about 40 kDa, from about 6 kDa to about 30 kDa, from about 8 kDa to about 18 kDa, from about 10 kDa to about 20 kDa, or from about 15 kDa to about 20 kDa. In some embodiments, the polymer (e.g., PLGA) has a weight average molecular weight of from about 1 kDa to about 70 kDa, from about 1 kDa to about 55 kDa, from about 1 kDa to about 50 kDa, from about 4 kDa to about 40 kDa, from about 5 kDa to about 25 kDa, from about 6 kDa to about 30 kDa, from about 8 kDa to about 18 kDa, from about 10 kDa to about 20 kDa, from about 15 kDa to about 55 kDa, or from about 15 kDa to about 20 kDa. In some embodiments, the polymer (e.g., PLGA) has a weight average molecular weight of greater than about 5 kDa, greater than about 10 kDa, greater than about 15 kDa, greater than about 16 kDa, or greater than about 18 kDa. In some embodiments, the polymer (e.g., PLGA) has a weight average molecular weight of greater than about 20 kDa, greater than about 25 kDa, greater than about 30 kDa, greater than about 40 kDa, greater than about 45 kDa, or greater than about 50 kDa.

As used herein, “weight average molecular weight” or “Mw” refers to the weighted average molecular weight of a polymer. It can be measured by any suitable means known in the art. In some embodiments, Mw is measured using gel permeation chromatography (GPC). Accordingly, in some embodiments, the polymer (e.g., PLGA) has a particular weight average molecular weight (e.g., as described herein) when measured using GPC. GPC is a column fractionation method wherein polymer molecules in solution are separated based on their size. The separated polymer molecules are detected by a detector to generate a GPC chromatogram, which is a plot of elution volume or time (related to molecular weight) versus abundance. A GPC chromatogram may be integrated to determine Mw. In some embodiments, Mw is measured using GPC according to the following exemplary procedure: GPC samples of polymer(s) of interest are dissolved in appropriate solvent, approximately 50 mg in 10 mL of solvent. Injections of 50-200 μL are made to generate chromatograms. Chromatograms may be generated using various systems. In some embodiments, a system comprises an Agilent LC 1100 with a refractive index detector using Chemstation software. In some embodiments, a system comprises a Waters 510 pump, a Shimadzu CTO-10A column oven, and a Waters 410 differential refractometer. Data may be recorded directly to a PC via a Polymer Labs data capture unit using Caliber® software. A calibration curve may be generated using polystyrene standards. Mw, Mn, and MWD relative to polystyrene are calculated. Representative solvents for use in GPC comprise: chloroform, dichloromethane (methylene chloride), and tetrahydrofuran (THF). Representative column sets comprise: (1) a PLgel MIXED guard column in series with two Polymer Labs Mixed C columns, (2) a PLgel MIXED guard column in series with two Polymer Labs Mixed D columns, or (3) two Polymer Labs Mesopore columns in series. Representative polystyrene calibrants comprise: Polymer Labs Easical PS1 kit, Polymer Labs Easical PS2 kit, Polymer Labs S-L-10 kit.

In some embodiments, provided compositions comprise about 1 wt %, about 2 wt %, about 5 wt %, about 8 wt %, about 10 wt %, about 15 wt %, about 20 wt %, about 30 wt %, or about 40 wt % polymer (e.g., PLGA), based on the weight of the vehicle or the total weight of the composition. In some embodiments, provided compositions comprise from about 1 wt % to about 40 wt %, about 2 wt % to about 30 wt %, about 3 wt % to about 20 wt %, or about 5 wt % to about 10 wt % polymer (e.g., PLGA), based on the weight of the vehicle or the total weight of the composition. In some embodiments, provided compositions comprise from about 1 wt % to about 40 wt %, about 2 wt % to about 30 wt %, about 3 wt % to about 20 wt %, about 5 wt % to about 30 wt %, about 10 wt % to about 25 wt %, about 5 wt % to about 20 wt %, or about 5 wt % to about 10 wt % polymer (e.g., PLGA), based on the weight of the vehicle or the total weight of the composition. While not wishing to be bound by any particular theory, in some embodiments, the amount of polymer is minimized in order to minimize formation of acid and/or other byproducts upon sterilization (e.g., with gamma irradiation) and/or to minimize acid formation in the body as the drug is released (e.g., while polymer is degrading).

Polymers described herein can be prepared using techniques that are generally known in the art. For example, polylactide can be prepared via initiation with a monoalcohol according to the following scheme:

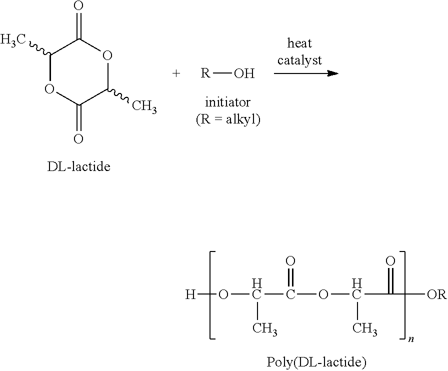

Similarly, poly(lactide)(glycolide) can be prepared via initiation with a monoalcohol according to the following scheme, wherein arrangement of monomers may be random, as opposed to being dimeric as depicted below:

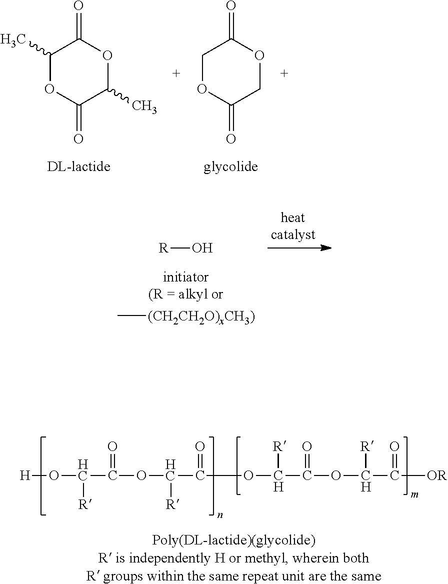

Alternatively, lactic acid-based polymers (e.g., polylactide) described herein can be prepared via initiation with a diol according to the following scheme:

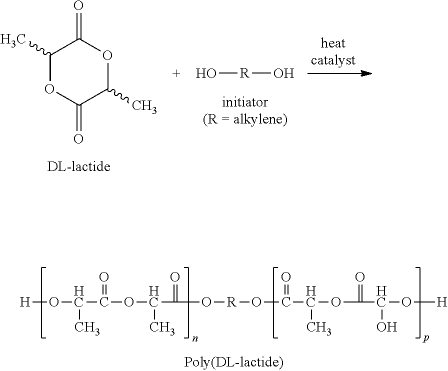

Alternatively, lactic acid-based polymers (e.g., polylactide) described herein can be prepared via initiation with water or a hydroxyl-containing carboxylic acid monomer according to the following scheme:

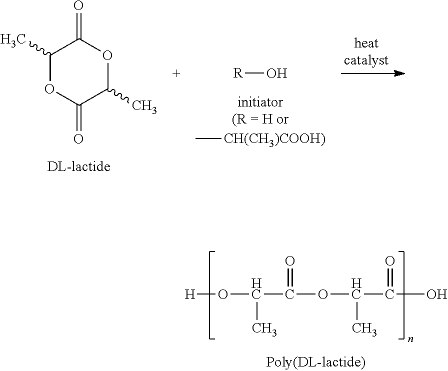

In some embodiments, the lactic acid-based polymer is prepared via initiation with an initiator selected from diols (such as 1,6-hexanediol, 1,2-propanediol, 1,3-propanediol, 1,4-butanediol, and the like), difunctionalized poly(ethyleneglycol)s (PEGs), monofunctionalized alcohols (such as 1-dodecanol, methyl lactate, ethyl lactate, and the like), monofunctional PEGs (such as methoxyPEG and the like), fatty alcohols, water, glycolic acid, lactic acid, and citric acid. In some embodiments, the initiator is a fatty alcohol or an acid. In some embodiments, the initiator is lactic acid. In some embodiments, the initiator is dodecanol (e.g., 1-dodecanol).

In some embodiments, the lactic acid-based polymer (e.g., PLGA) comprises an end group, depending on the method of preparation. In some embodiments, the end group is an alkoxy end group. In some embodiments, the alkoxy end group comprises or consists of 2 to 24 carbon atoms. In some embodiments, the alkoxy end group comprises or consists of 12 carbon atoms. In some embodiments, the end group is a hydroxy end group.

Without wishing to be bound by any particular theory, provided compositions comprising PLGA prepared via initiation with dodecanol (i.e., PLGA with an alkoxy end group comprising 12 carbon atoms) tend to exhibit desirable solubility properties. Thus, such compositions may require less solvent and/or may be more resistant to phase separation. Accordingly, in some embodiments, provided compositions comprise PLGA initiated with dodecanol (e.g., 1-dodecanol).

In some embodiments, provided compositions do not comprise cellulose acetate butyrate.

### Solvent

In some embodiments, the present disclosure provides compositions comprising a tenofovir agent, and further comprising a solvent. In some embodiments, provided compositions further comprise a solvent. In some embodiments, provided compositions further comprise a vehicle comprising a solvent. In some embodiments, provided compositions comprise a tenofovir agent and a vehicle comprising a solvent.

Without wishing to be bound by any particular theory, solvents suitable for use in provided compositions are often biocompatible, hydrophilic, water miscible, water soluble, and/or non-toxic. Suitable solvents do not cause significant tissue irritation or necrosis at the site of administration (e.g., injection or implantation) when used in conjunction with the present disclosure. Furthermore, suitable solvents are often water miscible and/or water soluble, so that they will diffuse into bodily fluids or other aqueous media. Additionally, the polymer (e.g., PLGA) and/or the HVLCM typically are soluble and/or miscible in the solvent.

In some embodiments, the solvent is or comprises an organic solvent. In some embodiments, the solvent is or comprises a polar solvent. In some embodiments, the solvent is or comprises a non-polar solvent. In some embodiments, the solvent is or comprises a hydrophilic solvent. In some embodiments, the solvent is or comprises a hydrophobic solvent.

In some embodiments, the solvent is or comprises one or more of N-methyl-pyrrolidone (NMP), dimethylsulfoxide (DMSO), propylene carbonate (PC), benzyl alcohol (BA), benzyl benzoate (BB), dimethylacetamide, caprylic/capric triglyceride, polyoxyethylene ester of 12-hydroxystearic acid, ethanol, ethyl lactate, glycofurol, propylene glycol, acetone, methyl acetate, ethyl acetate, methyl ethyl ketone, triacetin, dimethylformamide, tetrahydrofuran, caprolactam, caprolactone, decylmethylsulfoxide, oleic acid, tocopherol, linoleic acid, oleic acid, ricinoleic acid, pyrrolidone, diethyl phthalate, isopropylidene glycerol, tripropionin, and 1-dodecylazacycloheptan-2-one. In some embodiments, the solvent is or comprises one or more of N-methyl-pyrrolidone (NMP), dimethylsulfoxide (DMSO), propylene carbonate (PC), benzyl alcohol (BA), benzyl benzoate (BB), dimethylacetamide, caprylic/capric triglyceride, polyoxyethylene ester of 12-hydroxystearic acid, ethanol, ethyl lactate, glycofurol, propylene glycol, acetone, methyl acetate, ethyl acetate, methyl ethyl ketone, triacetin, dimethylformamide, tetrahydrofuran, caprolactam, caprolactone, decylmethylsulfoxide, oleic acid, tocopherol, linoleic acid, oleic acid, ricinoleic acid, pyrrolidone, diethyl phthalate, isopropylidene glycerol, and 1-dodecylazacycloheptan-2-one. In some embodiments, the solvent is or comprises one or more of NMP, DMSO, PC, BA, BB, ethanol, and glycofurol. In some embodiments, the solvent is or comprises one or more of NMP, DMSO, PC, BB, and ethanol.

In some embodiments, the solvent comprises propylene carbonate (PC). In some embodiments, the solvent is PC. In some embodiments, the solvent consists essentially of PC.

Without wishing to be bound by theory, the present disclosure encompasses the recognition that provided compositions that comprise a mixture of solvents may be useful for achieving certain desirable results (e.g., particular release profiles described herein). Accordingly, in some embodiments, the solvent comprises a solvent mixture (e.g., a mixture of two or more of NMP, DMSO, PC, BA, BB, dimethylacetamide, caprylic/capric triglyceride, polyoxyethylene ester of 12-hydroxystearic acid, ethanol, ethyl lactate, glycofurol, propylene glycol, acetone, methyl acetate, ethyl acetate, methyl ethyl ketone, triacetin, dimethylformamide, tetrahydrofuran, caprolactam, caprolactone, decylmethylsulfoxide, oleic acid, tocopherol, linoleic acid, oleic acid, ricinoleic acid, pyrrolidone, diethyl phthalate, isopropylidene glycerol, tripropionin, and 1-dodecylazacycloheptan-2-one).

In some embodiments, the solvent is or comprises propylene carbonate (PC) and one or more solvents selected from NMP, DMSO, BA, BB, dimethylacetamide, caprylic/capric triglyceride, polyoxyethylene ester of 12-hydroxystearic acid, ethanol, ethyl lactate, glycofurol, propylene glycol, acetone, methyl acetate, ethyl acetate, methyl ethyl ketone, triacetin, dimethylformamide, tetrahydrofuran, caprolactam, caprolactone, decylmethylsulfoxide, oleic acid, tocopherol, linoleic acid, oleic acid, ricinoleic acid, pyrrolidone, diethyl phthalate, isopropylidene glycerol, and 1-dodecylazacycloheptan-2-one. In some embodiments, the solvent is or comprises propylene carbonate (PC) and dimethylsulfoxide (DMSO). In some embodiments, the solvent is or comprises propylene carbonate (PC) and ethanol.

As described above, the HVLCM is typically soluble and/or miscible in the solvent suitable for use in provided compositions. For example, SAIB is not miscible with glycerol, corn oil, peanut oil, 1,2-propanediol, polyethylene glycol (PEG200), super refined sesame oil, and super refined peanut oil. Accordingly, in some embodiments, the solvent does not comprise one or more of glycerol, corn oil, peanut oil, 1,2-propanediol, polyethylene glycol (PEG200), super refined sesame oil, and super refined peanut oil.

In some embodiments, the solvent does not comprise an alcohol. For example, in some embodiments, the solvent does not comprise ethanol. In some embodiments, the solvent does not comprise benzyl alcohol. Thus, in some embodiments, the composition is substantially free of alcohol, ethanol, and/or benzyl alcohol.

In some embodiments, the solvent does not comprise NMP.

In some embodiments, provided compositions comprise about 5 wt %, about 10 wt %, about 15 wt %, about 20 wt %, about 25 wt %, about 30 wt %, about 40 wt %, about 50 wt %, about 80 wt %, or about 90 wt % solvent, based on the weight of the vehicle or the total weight of the composition. In some embodiments, provided compositions comprise about 5 wt %, about 10 wt %, about 15 wt %, about 20 wt %, about 25 wt %, about 30 wt %, about 35 wt %, about 40 wt %, about 45 wt %, about 50 wt %, about 55 wt %, about 80 wt %, or about 90 wt % solvent, based on the weight of the vehicle or the total weight of the composition. In some embodiments, provided compositions comprise from about 5 wt % to about 90 wt %, from about 10 wt % to about 90 wt %, from about 10 wt % to about 80 wt %, from about 10 wt % to about 60 wt %, from about 10 wt % to about 40 wt %, or from about 15 wt % to about 35 wt % solvent, based on the weight of the vehicle or the total weight of the composition. In some embodiments, provided compositions comprise from about 5 wt % to about 90 wt %, from about 10 wt % to about 90 wt %, from about 10 wt % to about 80 wt %, from about 10 wt % to about 60 wt %, from about 20 wt % to about 60 wt %, from about 25 wt % to about 55 wt %, from about 10 wt % to about 40 wt %, or from about 15 wt % to about 35 wt % solvent, based on the weight of the vehicle or the total weight of the composition.

In some embodiments, provided compositions comprise about 5 wt %, about 10 wt %, about 15 wt %, about 20 wt %, about 25 wt %, about 30 wt %, about 40 wt %, about 50 wt %, about 80 wt %, or about 90 wt % PC, based on the weight of the vehicle or the total weight of the composition. In some embodiments, provided compositions comprise about 5 wt %, about 10 wt %, about 15 wt %, about 20 wt %, about 25 wt %, about 30 wt %, about 35 wt %, about 40 wt %, about 45 wt %, about 50 wt %, about 55 wt %, about 80 wt %, or about 90 wt % PC, based on the weight of the vehicle or the total weight of the composition. In some embodiments, provided compositions comprise from about 5 wt % to about 90 wt %, from about 10 wt % to about 90 wt %, from about 10 wt % to about 80 wt %, from about 10 wt % to about 60 wt %, from about 10 wt % to about 40 wt %, or from about 15 wt % to about 35 wt % PC, based on the weight of the vehicle or the total weight of the composition. In some embodiments, provided compositions comprise from about 5 wt % to about 90 wt %, from about 10 wt % to about 90 wt %, from about 10 wt % to about 80 wt %, from about 10 wt % to about 60 wt %, from about 20 wt % to about 60 wt %, from about 25 wt % to about 55 wt %, from about 10 wt % to about 40 wt %, or from about 15 wt % to about 35 wt % PC, based on the weight of the vehicle or the total weight of the composition.

### Other Components:

In some embodiments, provided compositions optionally further comprise one or more additional components (i.e., additives) in order to modify the properties of the compositions as desired. The additives may be present in any amount that is sufficient to impart the desired properties. The amount of additive used will generally be a function of the nature of the additive and the effect to be achieved, and can be easily determined by one of skill in the art. For example, when present, additive(s) are typically present in provided compositions from about 0.1 wt % to about 20 wt %, based on the weight of the vehicle or the total weight of the composition.

In some embodiments, provided compositions further comprise a buffer, in order to, e.g., modify the pH of the composition.

In some embodiments, provided compositions further comprise one or more additional polymers (i.e., a polymer other than the lactic acid-based polymer), such as a non-biodegradable polymer. Non-limiting examples of such polymers include polyacrylates, ethylene-vinyl acetate polymers, cellulose and cellulose derivatives, acyl substituted cellulose acetates and derivatives thereof (such as cellulose acetate butyrate and cellulose acetate propionate), non-erodible polyurethanes, polystyrenes, polyvinyl chloride, polyvinyl fluoride, poly(vinyl imidazole), chlorosulphonated polyolefins, polyethylene oxide, polyethylene, polyvinyl pyrrolidone, ethylene vinylacetate, and polyethylene glycol.

In some embodiments, provided compositions further comprise one or more natural or synthetic oils and/or fats in order to, e.g., increase the hydrophobicity of provided compositions and thereby slowing degradation and/or water uptake of the composition. Exemplary suitable natural and synthetic oils include vegetable oil, peanut oil, medium chain triglycerides, almond oil, olive oil, sesame oil, peanut oil, fennel oil, camellia oil, corn oil, castor oil, cotton seed oil, soybean oil, either crude or refined, and medium chain fatty acid triglycerides. Exemplary suitable fats include lard and tallow.

In some embodiments, provided compositions further comprise one or more carbohydrates and/or carbohydrate derivatives. Non-limiting examples of carbohydrates and carbohydrate derivatives include monosaccharides (e.g., simple sugars such as fructose and glucose), disaccharides (such as sucrose, maltose, cellobiose, and lactose), and polysaccharides.

In some embodiments, provided compositions further comprise one or more preservatives (such as paraben derivatives, e.g., methyl paraben and propyl paraben), stabilizers, anti-oxidants (such as butyl hydroxyanisole, butyl hydroxytoluene, propyl gallate, vitamin E acetate, and purified hydroquinone), coloring agents, isotonic agents, humectants (such as sorbitol), sequesterants (such as citric acid), vitamins, vitamin precursors, and/or surfactants.

In some embodiments, provided compositions further comprise one or more viscosity enhancers, antioxidants, preservatives, and particle stabilizers. For instance, provided compositions may comprise one or more of ricinoleic acid, polyoxyethylene-polyoxypropylene block copolymer, polyvinylpyrrolidone, polyethyeleneglycol (e.g., PEG4000), and Cremophor EL® ethoxylated castor oil which includes polyethylene glycol ether.

The present disclosure also encompasses the recognition that it may be desirable to control or reduce water content in provided formulations. For example, if the presence of water increases the rate of polymer and/or active agent degradation, removing and/or minimizing the amount of water may be desirable. Accordingly, the present disclosure also provides compositions having surprisingly low water content. In some embodiments, provided compositions are substantially free of water. In some embodiments, provided compositions comprise less than about 0.5 wt %, less than about 0.35 wt %, less than about 0.25 wt %, less than about 0.2 wt %, less than about 0.15 wt %, less than about 0.1%, less than about 0.01 wt % or less than about 0.005 wt % water, based on the weight of the vehicle or the total weight of the composition. In some embodiments, provided compositions comprise from about 0.001 wt % to about 0.35 wt %, from about 0.001 wt % to about 0.25 wt %, from about 0.001 wt % to about 0.1 wt %, from about 0.001 wt % to about 0.01 wt %, or from about 0.001 wt % to about 0.005 wt % water, based on the weight of the vehicle or the total weight of the composition.

### Provided Compositions:

In some embodiments, provided compositions have a total weight of from about 25 mg to about 10,000 mg, from about 50 mg to about 5000 mg, from about 100 mg to about 4000 mg, from about 150 mg to about 3000 mg, or from about 200 mg to about 2000 mg. In some embodiments, provided compositions have a total weight of about 50 mg, about 100 mg, about 200 mg, about 300 mg, about 400 mg, about 500 mg, about 600 mg, about 700 mg, about 800 mg, about 900 mg, about 1000 mg, about 1500 mg, about 2000 mg, about 2500 mg, about 3000 mg, about 3500 mg, about 4000 mg, about 4500 mg, or about 5000 mg.

In some embodiments, provided compositions have a total volume of from about 0.025 mL to about 10 mL, from about 0.05 mL to about 5 mL, from about 0.1 mL to about 4 mL, from about 0.15 mL to about 3 mL, or from about 0.2 mL to about 2 mL. In some embodiments, provided compositions have a total volume of about 0.05 mL, about 0.1 mL, about 0.2 mL, about 0.3 mL, about 0.4 mL, about 0.5 mL, about 0.6 mL, about 0.7 mL, about 0.8 mL, about 0.9 mL, about 1 mL, about 1.5 mL, or about 2 mL.

In some embodiments, the present disclosure provides a composition comprising:
(i) from about 5 wt % to about 35 wt % tenofovir alafenamide sebacate, based on the weight of the vehicle or the total weight of the composition;
(ii) from about 5 wt % to about 20 wt % poly(lactic acid)(glycolic acid), based on the weight of the vehicle or the total weight of the composition;
(iii) from about 10 wt % to about 40 wt % propylene carbonate, based on the weight of the vehicle or the total weight of the composition; and
(iv) from about 40 wt % to about 60 wt % sucrose acetate isobutyrate, based on the weight of the vehicle or the total weight of the composition.

In some embodiments, the present disclosure provides a composition comprising:
(i) from about 5 wt % to about 15 wt % tenofovir alafenamide sebacate, based on the weight of the vehicle or the total weight of the composition;
(ii) from about 5 wt % to about 10 wt % poly(lactic acid)(glycolic acid), based on the weight of the vehicle or the total weight of the composition;
(iii) from about 20 wt % to about 30 wt % propylene carbonate, based on the weight of the vehicle or the total weight of the composition; and
(iv) from about 50 wt % to about 60 wt % sucrose acetate isobutyrate, based on the weight of the vehicle or the total weight of the composition.

In some embodiments, the present disclosure provides a composition comprising:
(i) from about 5 wt % to about 35 wt % tenofovir alafenamide sebacate, based on the weight of the vehicle or the total weight of the composition;
(ii) from about 5 wt % to about 30 wt % poly(lactic acid)(glycolic acid), based on the weight of the vehicle or the total weight of the composition;
(iii) from about 20 wt % to about 60 wt % propylene carbonate, based on the weight of the vehicle or the total weight of the composition; and
(iv) from about 25 wt % to about 65 wt % sucrose acetate isobutyrate, based on the weight of the vehicle or the total weight of the composition.

In some embodiments, the present disclosure provides a composition comprising, based on the total weight of the composition:
(i) about 11.1 wt % tenofovir alafenamide sebacate;
(ii) about 8.9 wt % poly(lactic acid)(glycolic acid);
(iii) about 24.9 wt % propylene carbonate; and
(iv) about 55.1 wt % sucrose acetate isobutyrate.

In some embodiments, the present disclosure provides a composition comprising:
(i) about 11.1 wt % tenofovir alafenamide sebacate, based on the total weight of the composition;
(ii) about 10 wt % poly(lactic acid)(glycolic acid) (e.g., PLGA with a weight average molecular weight of about 18 kDa, e.g., when measured using GPC, and/or with lactic acid repeat units and glycolic acid repeat units in a molar ratio of about 90:10 and/or initiated with 1-dodecanol), based on the weight of the vehicle;
(iii) about 28 wt % propylene carbonate, based on the weight of the vehicle; and
(iv) about 62 wt % sucrose acetate isobutyrate, based on the weight of the vehicle.

In some embodiments, the present disclosure provides a composition comprising:
(i) about 11.1 wt % tenofovir alafenamide sebacate, based on the total weight of the composition;
(ii) about 19 wt % poly(lactic acid)(glycolic acid) (e.g., PLGA with a weight average molecular weight of about 8 kDa, e.g., when measured using GPC, and/or with lactic acid repeat units and glycolic acid repeat units in a molar ratio of about 75:25 and/or initiated with 1-dodecanol), based on the weight of the vehicle;
(iii) about 37 wt % propylene carbonate, based on the weight of the vehicle; and
(iv) about 44 wt % sucrose acetate isobutyrate, based on the weight of the vehicle.

In some embodiments, the present disclosure provides a composition comprising:
(i) about 22.2 wt % tenofovir alafenamide sebacate, based on the total weight of the composition;
(ii) about 19 wt % poly(lactic acid)(glycolic acid) (e.g., PLGA with a weight average molecular weight of about 8 kDa, e.g., when measured using GPC, and/or with lactic acid repeat units and glycolic acid repeat units in a molar ratio of about 75:25 and/or initiated with 1-dodecanol), based on the weight of the vehicle;
(iii) about 37 wt % propylene carbonate, based on the weight of the vehicle; and
(iv) about 44 wt % sucrose acetate isobutyrate, based on the weight of the vehicle.

In some embodiments, the present disclosure provides a composition comprising:
(i) about 11.1 wt % tenofovir alafenamide sebacate, based on the total weight of the composition;
(ii) about 20 wt % poly(lactic acid)(glycolic acid) (e.g., PLGA with a weight average molecular weight of about 48 kDa, e.g., when measured using GPC, and/or with lactic acid repeat units and glycolic acid repeat units in a molar ratio of about 65:35 and/or initiated with 1-dodecanol), based on the weight of the vehicle;
(iii) about 55 wt % propylene carbonate, based on the weight of the vehicle; and
(iv) about 25 wt % sucrose acetate isobutyrate, based on the weight of the vehicle.

In some embodiments, the present disclosure provides a composition comprising:
(i) about 11.1 wt % tenofovir alafenamide sebacate, based on the total weight of the composition;
(ii) about 25 wt % poly(lactic acid)(glycolic acid) (e.g., PLGA with a weight average molecular weight of about 18 kDa, e.g., when measured using GPC, and/or with lactic acid repeat units and glycolic acid repeat units in a molar ratio of about 90:10 and/or initiated with 1-dodecanol), based on the weight of the vehicle;
(iii) about 37 wt % propylene carbonate, based on the weight of the vehicle; and
(iv) about 38 wt % sucrose acetate isobutyrate, based on the weight of the vehicle.

In some embodiments, the present disclosure provides a composition comprising:
(i) about 11.1 wt % tenofovir alafenamide sebacate, based on the total weight of the composition;
(ii) about 20 wt % poly(lactic acid)(glycolic acid) (e.g., PLGA with a weight average molecular weight of about 18 kDa, e.g., when measured using GPC, and/or with lactic acid repeat units and glycolic acid repeat units in a molar ratio of about 90:10 and/or initiated with 1-dodecanol), based on the weight of the vehicle;
(iii) about 9 wt % dimethylsulfoxide, based on the weight of the vehicle;
(iv) about 21 wt % propylene carbonate, based on the weight of the vehicle; and
(v) about 50 wt % sucrose acetate isobutyrate, based on the weight of the vehicle.

In some embodiments, the present disclosure provides a composition comprising:
(i) about 11.1 wt % tenofovir alafenamide sebacate, based on the total weight of the composition;
(ii) about 20 wt % poly(lactic acid)(glycolic acid) (e.g., PLGA with a weight average molecular weight of about 18 kDa, e.g., when measured using GPC, and/or with lactic acid repeat units and glycolic acid repeat units in a molar ratio of about 90:10 and/or initiated with 1-dodecanol), based on the weight of the vehicle;
(iii) about 5.5 wt % ethanol, based on the weight of the vehicle;
(iv) about 21.5 wt % propylene carbonate, based on the weight of the vehicle; and
(v) about 53 wt % sucrose acetate isobutyrate, based on the weight of the vehicle.

In some embodiments, the present disclosure provides a composition comprising:
(i) about 11.1 wt % tenofovir alafenamide sebacate, based on the total weight of the composition;
(ii) about 20 wt % poly(lactic acid)(glycolic acid) (e.g., PLGA with a weight average molecular weight of about 40 kDa, e.g., when measured using GPC, and/or with lactic acid repeat units and glycolic acid repeat units in a molar ratio of about 75:25 and/or initiated with 1-dodecanol), based on the weight of the vehicle;
(iii) about 46 wt % propylene carbonate, based on the weight of the vehicle; and
(iv) about 34 wt % sucrose acetate isobutyrate, based on the weight of the vehicle.

In some embodiments, the present disclosure provides a composition comprising:
(i) about 11.1 wt % tenofovir alafenamide sebacate, based on the total weight of the composition;
(ii) about 20 wt % poly(lactic acid)(glycolic acid) (e.g., PLGA with a weight average molecular weight of about 51 kDa, e.g., when measured using GPC, and/or with lactic acid repeat units and glycolic acid repeat units in a molar ratio of about 75:25 and/or initiated with 1-dodecanol), based on the weight of the vehicle;
(iii) about 55 wt % propylene carbonate, based on the weight of the vehicle; and
(iv) about 25 wt % sucrose acetate isobutyrate, based on the weight of the vehicle.

In some embodiments, the present disclosure provides a composition comprising:
(i) about 11.1 wt % tenofovir alafenamide sebacate, based on the total weight of the composition;
(ii) about 20 wt % poly(lactic acid)(glycolic acid) (e.g., PLGA with a weight average molecular weight of about 29 kDa, e.g., when measured using GPC, and/or with lactic acid repeat units and glycolic acid repeat units in a molar ratio of about 75:25 and/or initiated with 1-dodecanol), based on the weight of the vehicle;
(iii) about 43 wt % propylene carbonate, based on the weight of the vehicle; and
(iv) about 37 wt % sucrose acetate isobutyrate, based on the weight of the vehicle.

In some embodiments, the present disclosure provides a composition comprising:
(i) from about 1 mg to about 500 mg tenofovir alafenamide sebacate;
(ii) from about 1 mg to about 500 mg poly(lactic acid)(glycolic acid);
(iii) from about 5 mg to about 1000 mg propylene carbonate; and
(iv) from about 10 mg to about 2000 mg sucrose acetate isobutyrate.

In some embodiments, the present disclosure provides a composition of Table 1A, Table 1B, Table 4A, or Table 4B below.

### Characteristics of Provided Compositions

As described above, the present disclosure provides novel long-acting formulations of a tenofovir agent. Provided compositions achieve certain desirable characteristics, as described herein.

For example, without wishing to be bound by any particular theory, compositions (e.g., formulations and/or vehicles provided herein) that are monophasic are easy to store, are stable, and/or enable consistent administration of the active agent. Monophasic compositions are particularly desirable to avoid inconsistencies related to administration of the composition. For instance, if a composition requires re-mixing because phase separation occurs after storage for a period of time, some subjects may not receive the same amount of each component of a provided composition, which may result in suboptimal outcomes, e.g., release profiles. Accordingly, in some embodiments, provided formulations are monophasic. In some embodiments, provided formulations comprise suitable amounts of each component (e.g., tenofovir agent, HVLCM, polymer, and/or solvent), so that the formulation is monophasic. In some embodiments, provided vehicles are monophasic. In some embodiments, provided vehicles comprise suitable amounts of each component (e.g., HVLCM, polymer, and/or solvent), so that the vehicle is monophasic.

Phase separation may be investigated by visual techniques well known to those skilled in the art. Some compositions may be rendered into a uniform clear solution by sufficient heating and mixing. Yet, when cooled to room temperature, two clear liquid phases may form. Sometimes, two clear layers may not be easy to detect, thus requiring strong light and thorough inspection to discern the boundary between the two phases. In some cases, compositions may appear clear and uniform on initial cooling to room temperature, but when left quiescent at room temperature for a period of time, the compositions may separate into two phases. In some cases, the composition may turn cloudy and slowly separate into two phases.

In some embodiments, provided formulations remain monophasic over a period of time (i.e., no phase separation of provided formulations is observed over a period of time). In some embodiments, provided formulations are monophasic after storage for 1 week, 2 weeks, 1 month, 2 months, 6 months, or longer. In some embodiments, provided formulations are monophasic after storage at 0° C., 10° C., 25° C., 37° C., or cooler, or warmer. In some embodiments, provided formulations are monophasic after storage at 0° C., 10° C., 25° C., 37° C., or cooler, or warmer for 1 week, 2 weeks, 1 month, 2 months, 6 months, or longer.

In some embodiments, provided vehicles remain monophasic over a period of time (i.e., no phase separation of provided vehicles is observed over a period of time). In some embodiments, provided vehicles are monophasic after storage for 1 week, 2 weeks, 1 month, 2 months, 6 months, or longer. In some embodiments, provided vehicles are monophasic after storage at −20° C., −10° C., 0° C., 10° C., 25° C., 37° C., or cooler, or warmer. In some embodiments, provided vehicles are monophasic after storage at −20° C., −10° C., 0° C., 10° C., 25° C., 37° C., or cooler, or warmer for 1 week, 2 weeks, 1 month, 2 months, 6 months, or longer.

In some instances, provided formulations comprising a tenofovir agent are not monophasic (e.g., are suspensions). Without wishing to be bound by any particular theory, formulations in which the tenofovir agent is not fully soluble in the vehicle may also be useful as long-acting formulations. In fact, such suspensions may enable even slower release of the tenofovir agent, which upon administration will need to dissolve first before releasing into the body. Accordingly, in some embodiments, provided compositions are suspensions. In some embodiments, provided compositions are suspensions of the tenofovir agent in the vehicle. In some embodiments, provided compositions are suspensions of the tenofovir agent in the vehicle, wherein the vehicle is monophasic.

Without wishing to be bound by any particular theory, a viscosity of provided compositions is within a desirable range, in order to, e.g., be easily administered through a needle or other suitable means for administration while still achieving desirable long-acting release characteristics. Accordingly, in some embodiments, provided compositions have a viscosity of less than about 20,000 cP, less than about 10,000 cP, less than about 8,000 cP, less than about 6,000 cP, less than about 4,000 cP, or less than about 2,000 cP at a shear rate of 500 s⁻¹ at 25° C. In some embodiments, provided compositions have a viscosity of less than about 10,000 cP, less than about 8,000 cP, less than about 6,000 cP, less than about 4,000 cP, or less than about 2,000 cP at a shear rate of 500 s⁻¹ at 25° C. In some embodiments, provided compositions have a viscosity from about 50 cP to about 10,000 cP, from about 500 cP to about 8,000 cP, from about 500 cP to about 6,000 cP, or from about 1,000 cP to about 10,000 cP at a shear rate of 500 s⁻¹ at 25° C. In some embodiments, provided compositions have a viscosity from about 10 cP to about 20,000 cP, from about 50 cP to about 10,000 cP, from about 500 cP to about 8,000 cP, from about 500 cP to about 6,000 cP, or from about 1,000 cP to about 10,000 cP at a shear rate of 500 s⁻¹ at 25° C.

Vehicle viscosity is related to the viscosity of provided formulations. In some embodiments, provided vehicles have a viscosity of less than about 10,000 cP, less than about 8,000 cP, less than about 6,000 cP, less than about 4,000 cP, or less than about 2,000 cP at a shear rate of 500 s⁻¹ at 25° C. In some embodiments, provided vehicles have a viscosity from about 50 cP to about 10,000 cP, from about 100 cP to about 8,000 cP, from about 200 cP to about 6,000 cP, or from about 500 cP to about 2,000 cP at a shear rate of 500 s⁻¹ at 25° C. In some embodiments, provided vehicles have a viscosity from about 10 cP to about 10,000 cP, from about 50 cP to about 10,000 cP, from about 100 cP to about 8,000 cP, from about 200 cP to about 6,000 cP, or from about 500 cP to about 2,000 cP at a shear rate of 500 s⁻¹ at 25° C.

In some embodiments, provided compositions are surprisingly shear-thinning (i.e., provided compositions have lower viscosities at higher shear, compared to viscosity at lower shear or no shear).

Additionally or alternatively, provided compositions can be injected within a suitable amount of time (e.g., within seconds) under a suitable amount of force, which is desirable, e.g., for convenient administration. For example, in some embodiments, provided compositions can be injected in less than about 20 seconds, less than about 15 seconds, less than about 13 seconds, less than about 10 seconds, or less than about 8 seconds with 5 lbf. In some embodiments, provided compositions can be injected in less than about 60 seconds, less than about 50 seconds, less than about 40 seconds, less than about 30 seconds, less than about 20 seconds, less than about 15 seconds, less than about 13 seconds, less than about 10 seconds, or less than about 8 seconds with 5 lbf. In some embodiments, provided compositions can be injected within from about 1 second to about 30 seconds, from about 2 seconds to about 20 seconds, from about 4 seconds to about 15 seconds, from about 6 seconds to about 12 seconds, or from about 6 seconds to about 10 seconds with 5 lbf. In some embodiments, provided compositions can be injected within from about 1 second to about 60 seconds, from about 1 second to about 30 seconds, from about 2 seconds to about 20 seconds, from about 4 seconds to about 15 seconds, from about 6 seconds to about 12 seconds, or from about 6 seconds to about 10 seconds with 5 lbf. In some embodiments, provided compositions can be injected within from about 1 second to about 30 seconds, from about 2 seconds to 20 seconds, from about 4 seconds to about 15 seconds, or from about 6 seconds to about 10 seconds with 10 lbf. In some embodiments, provided compositions can be injected within from about 1 second to about 60 seconds, from about 1 second to about 30 seconds, from about 2 seconds to 20 seconds, from about 4 seconds to about 15 seconds, or from about 6 seconds to about 10 seconds with 10 lbf.

Furthermore, it is desirable for provided compositions to be stable to storage for a period of time. In some embodiments, provided compositions comprise at least about 90%, about 95%, about 97%, about 98%, about 99%, or greater of the tenofovir agent after storage for 1 week, 2 weeks, 1 month, 2 months, 6 months, or longer at 0° C., 10° C., 25° C., 37° C., or cooler, or warmer, relative to the initial amount of the tenofovir agent before storage. In some embodiments, provided compositions comprise no more than about 10%, about 5%, about 3%, about 2%, about 1%, or less of total degradation products after storage for 1 week, 2 weeks, 1 month, 2 months, 6 months, or longer at 0° C., 10° C., 25° C., 37° C., or cooler, or warmer, relative to the initial amount of the total degradation products before storage.

As described above, provided compositions are useful as long-acting formulations. Accordingly, in some embodiments, upon administration of a provided composition to a subject or across a population of subjects, a therapeutically effective concentration of active agent is maintained (i.e., concentration of active agent is above a minimum threshold concentration (Cmin)) for a sufficient period of time.

In some embodiments, when administered subcutaneously as a single dose to a subject, provided compositions achieve a plasma tenofovir alafenamide concentration of greater than about 0.01 ng/mL, about 0.1 ng/mL, or about 0.5 ng/mL for at least about 10 days, about 20 days, about 25 days, about 30 days, about 35 days, about 40 days, about 45 days, about 50 days, about 55 days, about 60 days, about 65 days, or longer. In some embodiments, when administered subcutaneously as a single dose, provided compositions achieve a plasma tenofovir alafenamide concentration of greater than about 0.01 ng/mL, about 0.1 ng/mL, or about 0.5 ng/mL for about 10 days to about 75 days, about 20 days to about 70 days, about 20 days to about 40 days, about 25 days to about 35 days, about 30 days to about 65 days, about 40 days to about 60 days, or about 50 days to about 60 days.

In some embodiments, when administered subcutaneously as a single dose to a population of subjects, provided compositions have been established to achieve a mean or median plasma tenofovir alafenamide concentration of greater than about 0.01 ng/mL, about 0.1 ng/mL, or about 0.5 ng/mL for at least about 10 days, about 20 days, about 25 days, about 30 days, about 35 days, about 40 days, about 45 days, about 50 days, about 55 days, about 60 days, about 65 days, or longer. In some embodiments, when administered subcutaneously as a single dose to a population of subjects, provided compositions have been established to achieve a mean or median plasma tenofovir alafenamide concentration of greater than about 0.01 ng/mL, about 0.1 ng/mL, or about 0.5 ng/mL for about 10 days to about 75 days, about 20 days to about 70 days, about 20 days to about 40 days, about 25 days to about 35 days, about 30 days to about 65 days, about 40 days to about 60 days, or about 50 days to about 60 days.

In some embodiments, when administered subcutaneously as a single dose to a subject, provided compositions achieve an intracellular tenofovir diphosphate concentration in peripheral blood mononuclear cells greater than about 10 nM for at least about 10 days, about 20 days, about 25 days, about 30 days, about 35 days, about 40 days, about 45 days, about 50 days, about 55 days, about 60 days, about 65 days, or longer. In some embodiments, when administered subcutaneously as a single dose, provided compositions achieve an intracellular tenofovir diphosphate concentration in peripheral blood mononuclear cells greater than about 10 nM for about 10 days to about 75 days, about 20 days to about 70 days, about 30 days to about 65 days, about 45 days to about 55 days, about 40 days to about 60 days, or about 50 days to about 60 days.

In some embodiments, when administered subcutaneously as a single dose to a population of subjects, provided compositions have been established to achieve a mean or median intracellular tenofovir diphosphate concentration in peripheral blood mononuclear cells greater than about 10 nM for at least about 10 days, about 20 days, about 25 days, about 30 days, about 35 days, about 40 days, about 45 days, about 50 days, about 55 days, about 60 days, about 65 days, or longer. In some embodiments, when administered subcutaneously as a single dose to a population of subjects, provided compositions have been established to achieve a mean or median intracellular tenofovir diphosphate concentration in peripheral blood mononuclear cells greater than about 10 nM for about 10 days to about 75 days, about 20 days to about 70 days, about 30 days to about 65 days, about 45 days to about 55 days, about 40 days to about 60 days, or about 50 days to about 60 days.

In some embodiments, when administered subcutaneously as a single dose to a subject, provided compositions achieve a therapeutically effective plasma concentration of the tenofovir agent, or a metabolite thereof (e.g. tenofovir diphosphate), for at least about 7 days, about 14 days, about 21 days, about 28 days, or more.

In some embodiments, when administered subcutaneously as a single dose to a subject, provided compositions achieve a therapeutically effective intracellular concentration in peripheral blood mononuclear cells of the tenofovir agent, or a metabolite thereof (e.g., tenofovir diphosphate), for at least about 7 days, about 14 days, about 21 days, about 28 days, or more.

In some embodiments, upon administration, provided compositions achieve a slower release of tenofovir agent when compared to a reference composition. In some such embodiments, the reference composition is a tablet comprising 25 mg tenofovir alafenamide administered once daily.

In some embodiments, upon administration, provided compositions achieve a therapeutically effective concentration of tenofovir agent comparable to that of a reference composition. In some such embodiments, the reference composition is a tablet comprising 25 mg tenofovir alafenamide administered once daily.

In some embodiments, when a provided composition is placed in phosphate-buffered saline at 37° C. (e.g., at pH 6.0 or 7.4), the amount of tenofovir agent released from the provided composition after 4 weeks is about 20%, about 30%, about 40%, about 50%, about 60%, about 70%, about 80%, about 90%, or about 100% of the total amount of tenofovir agent in the provided composition. In some embodiments, when a provided composition is placed in either (1) phosphate-buffered saline at 37° C. (e.g., at pH 6.0 or 7.4) for 4 weeks or (2) phosphate-buffered saline at 37° C. (e.g., at pH 6.0 or 7.4) for 10 days and then 20 mM KH₂PO₄, pH 6.0, with 0.9% NaCl buffer for 18 days, the amount of tenofovir agent released from the provided composition is from about 20% to about 100%, about 20% to about 80%, about 40% to about 100%, about 50% to about 100%, or about 40% to about 80% of the total amount of tenofovir agent in the provided composition.

In some embodiments, the present disclosure encompasses the recognition that provided compositions that achieve a release profile that is not characterized by an initial burst release of active agent (e.g., an initial burst within the first 24 hours, 48 hours, or 72 hours of administration). In some embodiments, when a provided composition is placed in phosphate-buffered saline at 37° C. (e.g., at pH 6.0 or 7.4), the amount of tenofovir agent released from the provided composition after 2 days is less than about 10%, less than about 8%, or less than about 5% of the total amount of tenofovir agent in the provided composition. In some embodiments, when a provided composition is placed in phosphate-buffered saline at 37° C. (e.g., at pH 6.0 or 7.4), the amount of tenofovir agent released from the provided composition after 1 week is less than about 20%, less than about 15%, or less than about 10% of the total amount of tenofovir agent in the provided composition.

In some embodiments, when a provided composition is placed in phosphate-buffered saline at 37° C. (e.g., at pH 6.0 or 7.4), the amount of tenofovir agent released from the provided composition after 24 hours is less than about 40%, less than about 30%, less than about 20%, or less than about 10% of the amount released after 28 days. In some embodiments, when a provided composition is placed in phosphate-buffered saline at 37° C. (e.g., at pH 6.0 or 7.4), the amount of tenofovir agent released from the provided composition after 24 hours is less than about 50%, less than about 40%, less than about 30%, less than about 20%, or less than about 10% of the amount released after 28 days.

In some embodiments, when a provided composition is placed in phosphate-buffered saline at 37° C. (e.g., at pH 6.0 or 7.4), the amount of tenofovir agent released from the provided composition after 28 days is greater than about 30%, greater than about 40%, greater than 50%, greater than about 60%, greater than about 70%, or greater than about 80% of a total amount of active agent in the composition. In some embodiments, when a provided composition is placed in phosphate-buffered saline at 37° C. (e.g., at pH 6.0 or 7.4), the amount of tenofovir agent released from the provided composition after 28 days is greater than about 20%, greater than about 30%, greater than about 40%, greater than 50%, greater than about 60%, greater than about 70%, or greater than about 80% of a total amount of active agent in the composition.

### Methods of Preparing Provided Compositions

The present disclosure also provides methods of manufacturing provided compositions. In some embodiments, a method of manufacturing a provided composition comprises:
(i) providing a vehicle comprising a high viscosity liquid carrier material (HVLCM); and
(ii) combining the vehicle with a tenofovir agent under suitable conditions to give the provided composition.

In some embodiments, the method comprises combining the vehicle with a tenofovir agent under suitable conditions, wherein the suitable conditions comprise combining the vehicle with the tenofovir agent with stirring (e.g., stirring with a stir bar, an overhead stirrer, or a homogenizer). In some embodiments, the tenofovir agent is added to the vehicle (e.g., in a controlled manner). In some embodiments, the method further comprises homogenizing the mixture of tenofovir agent and vehicle (e.g., in order to obtain a uniform dispersion). In some embodiments, the tenofovir agent is provided and/or utilized in crystalline form (e.g., tenofovir alafenamide sebacate Form I).

In some embodiments, the vehicle comprises a HVLCM, a polymer, and a solvent. Accordingly, in some embodiments, the method further comprises mixing the HVLCM, the polymer, and the solvent under suitable conditions. In some such embodiments, suitable conditions comprise a suitable temperature of about 25° C., about 30° C., about 40° C., about 50° C., about 60° C., or about 70° C., or any range therein. In some such embodiments, suitable conditions comprise a suitable period of time of about 30 min, about 1 h, about 2 h, about 3 h, about 4 h, about 8 h, about 16 h, about 24 h, or about 48 h, or any range therein. In some such embodiments, suitable conditions comprise a suitable mixing speed of about 5 rpm, about 10 rpm, about 20 rpm, about 25 rpm, or about 30 rpm.

In some embodiments, the method further comprises mixing the polymer and the solvent, e.g., before combining with the HVLCM. In some embodiments, the method further comprises mixing the polymer and the solvent under suitable conditions. In some such embodiments, suitable conditions comprise a suitable temperature of about 10° C., about 20° C., about 25° C., about 30° C., about 40° C., or about 50° C., or any range therein. In some such embodiments, suitable conditions comprise a suitable period of time of about 30 min, about 1 h, about 2 h, about 3 h, about 4 h, about 8 h, about 12 h, about 16 h, or about 24 h, or any range therein. In some such embodiments, suitable conditions comprise a suitable mixing speed of about 5 rpm, about 10 rpm, about 20 rpm, about 25 rpm, or about 30 rpm. In some embodiments, the method further comprises allowing the polymer to warm to room temperature before combining with the solvent.

In some embodiments, the method further comprises heating the HVLCM before combining with the polymer and the solvent. In some such embodiments, the HVLCM is heated to about 50° C., about 60° C., about 70° C., about 80° C., about 90° C., or about 100° C., or any range therein.

In some embodiments, the method further comprises allowing the vehicle to cool to room temperature before combining with the tenofovir agent.

In some embodiments, the tenofovir agent is milled before combining with the vehicle. In some embodiments, the tenofovir agent is dissolved in the composition. In some embodiments, the tenofovir agent is suspended in the composition. In some embodiments, the tenofovir agent has a median particle size, as measured by laser diffraction, from about 0.1 μm to about 100 μm, about 0.2 μm to about 90 μm, about 0.25 μm to about 80 μm, about 0.5 μm to about 70 μm, about 1 μm to about 70 μm, about 2 μm to about 60 μm, about 5 μm to about 60 μm, about 10 μm to about 50 μm, or about 10 μm to about 40 μm.

In some embodiments, the method further comprises removing water so that the provided composition comprises less than about 0.5 wt %, less than about 0.35 wt %, less than about 0.25 wt %, less than about 0.2 wt %, less than about 0.15 wt %, less than about 0.1%, less than about 0.01 wt % or less than about 0.005 wt % water, based on the weight of the vehicle or the total weight of the composition. In some embodiments, the method further comprises removing water so that the provided composition comprises from about 0.001 wt % to about 0.35 wt %, from about 0.001 wt % to about 0.25 wt %, from about 0.001 wt % to about 0.1 wt %, from about 0.001 wt % to about 0.01 wt %, or from about 0.001 wt % to about 0.005 wt % water based on the weight of the vehicle or the total weight of the composition. In some embodiments, the method further comprises removing the water under an inert gas (e.g., nitrogen). In some embodiments, the method further comprises removing the water by heating and/or mixing the mixture.

In some embodiments, the method further comprises sterilizing the provided composition. In some embodiments, the method further comprises sterilizing the provided composition with gamma irradiation. In some embodiments, the gamma irradiation dose is less than about 25 kGy, less than about 20 kGy, less than about 15 kGy, or less than about 10 kGy. In some embodiments, the gamma irradiation dose is from about 10 kGy to about 25 kGy, about 15 kGy to about 25 kGy, about 15 kGy to about 20 kGy, or about 20 kGy to about 25 kGy.

In some embodiments, the method comprises irradiating the tenofovir agent before combining the tenofovir agent with the vehicle. In some embodiments, the method comprises filter sterilizing the vehicle before combining the vehicle with the tenofovir agent. In some embodiments, the method comprises combining the tenofovir agent (e.g., the tenofovir agent that has been irradiated) with the vehicle (e.g., the vehicle that has been filter sterilized) under aseptic conditions.

In some embodiments, the provided composition comprises at least 95%, at least 97%, at least 98%, at least 99%, or more of the tenofovir agent after gamma irradiation, relative to the initial amount of the tenofovir agent before gamma irradiation. In some embodiments, the provided composition comprises less than about 5%, less than about 3%, less than about 2%, less than about 1%, or less of total degradation products after gamma irradiation. In some embodiments, the provided composition comprises no more than about 5%, no more than about 3%, no more than about 2%, no more than about 1% or less of additional degradation products after gamma irradiation, relative to the initial amount of total degradation products before gamma irradiation.

### Uses of Provided Compositions:

Provided herein are methods of using provided compositions. In some embodiments, the present disclosure provides a method of administering a therapeutically effective dose of a tenofovir agent to a subject in need thereof, the method comprising administering to the subject a composition or dosage form provided herein.

In some embodiments, a method comprises administering a provided composition, such that the administration achieves one or more of the following characteristics in a subject:
(1) plasma tenofovir alafenamide concentration greater than about 0.01 ng/mL, about 0.1 ng/mL, or about 0.5 ng/mL for at least about 10 days, about 20 days, about 25 days, about 30 days, about 35 days, about 40 days, about 45 days, about 50 days, about 55 days, about 60 days, about 65 days, or longer; and
(2) intracellular tenofovir diphosphate concentration in peripheral blood mononuclear cells greater than about 10 nM for at least about 10 days, about 20 days, about 25 days, about 30 days, about 35 days, about 40 days, about 45 days, about 50 days, about 55 days, about 60 days, about 65 days, or longer.

In some embodiments, a method comprises administering a provided composition, wherein the provided composition, when administered subcutaneously to a population of subjects, has been established to achieve one or more of the following characteristics:
(1) a mean or median plasma tenofovir alafenamide concentration greater than about 0.01 ng/mL, about 0.1 ng/mL, or about 0.5 ng/mL for at least about 10 days, about 20 days, about 25 days, about 30 days, about 35 days, about 40 days, about 45 days, about 50 days, about 55 days, about 60 days, about 65 days, or longer; and
(2) a mean or median intracellular tenofovir diphosphate concentration in peripheral blood mononuclear cells greater than about 10 nM for at least about 10 days, about 20 days, about 25 days, about 30 days, about 35 days, about 40 days, about 45 days, about 50 days, about 55 days, about 60 days, about 65 days, or longer.

In some embodiments, a method comprises administering a provided composition, wherein the provided composition has been established to achieve one or more of the following characteristics in a dog subject (e.g., a male beagle dog subject):
(1) plasma tenofovir alafenamide concentration greater than about 0.01 ng/mL, about 0.1 ng/mL, or about 0.5 ng/mL for at least about 10 days, about 20 days, about 25 days, about 30 days, about 35 days, about 40 days, about 45 days, about 50 days, about 55 days, about 60 days, about 65 days, or longer; and
(2) intracellular tenofovir diphosphate concentration in peripheral blood mononuclear cells greater than about 10 nM for at least about 10 days, about 20 days, about 25 days, about 30 days, about 35 days, about 40 days, about 45 days, about 50 days, about 55 days, about 60 days, about 65 days, or longer.

In some embodiments, a method comprises administering a provided composition, wherein the provided composition has been established to achieve one or more of the following characteristics in a population of dog subjects (e.g., a population of male beagle dog subjects):
(1) a mean or median plasma tenofovir alafenamide concentration greater than about 0.01 ng/mL, about 0.1 ng/mL, or about 0.5 ng/mL for at least about 10 days, about 20 days, about 25 days, about 30 days, about 35 days, about 40 days, about 45 days, about 50 days, about 55 days, about 60 days, about 65 days, or longer; and
(2) a mean or median intracellular tenofovir diphosphate concentration in peripheral blood mononuclear cells greater than about 10 nM for at least about 10 days, about 20 days, about 25 days, about 30 days, about 35 days, about 40 days, about 45 days, about 50 days, about 55 days, about 60 days, about 65 days, or longer.

In some embodiments, the present disclosure provides a method of treating and/or preventing a viral infection in a subject in need thereof, comprising administering to the subject a composition or dosage form provided herein.

### HIV Infection

The present disclosure provides methods of treating and/or preventing human immunodeficiency virus (HIV) infection in a subject in need thereof. In some embodiments, a method of treating and/or preventing HIV infection in a subject in need thereof comprises administering to the subject a composition provided herein. In some embodiments, the method is for treating and/or preventing HIV-1 infection. In some embodiments, the method is for treating and/or preventing HIV-2 infection.

In some embodiments, a method of treating HIV infection in a subject in need thereof comprises administering to the subject a composition provided herein. In some such embodiments, the subject is HIV positive. In some such embodiments, the subject is of unknown HIV status. In some such embodiments, the subject is not HIV negative.

In some embodiments, a method of preventing HIV infection in a subject in need thereof comprises administering to the subject a composition provided herein. In some such embodiments, the subject is HIV negative. In some embodiments, the subject is at risk of acquiring HIV infection.

In some embodiments, the present disclosure provides compositions for use in the treatment and/or prevention of HIV infection in a subject.

In some embodiments, the present disclosure provides compositions for the manufacture of a medicament for treating and/or preventing HIV infection in a subject.

In some embodiments, the present disclosure provides a method of treating and/or preventing HIV infection in a subject in need thereof, comprising administering to the subject a combination therapy comprising a composition provided herein and one or more (e.g., one, two, three, one or two, or one to three) additional therapeutic agents. In some embodiments, a method for treating an HIV infection in a human subject having or at risk of having the infection is provided, comprising administering to the human subject a therapeutically effective amount of a composition disclosed herein, in combination with a therapeutically effective amount of one or more (e.g., one, two, three, one or two, or one to three) additional therapeutic agents. In some embodiments, the subject is receiving or has received one or more additional therapeutic agents. In some embodiments, the additional therapeutic agents are selected from the same class of therapeutic agents. In some embodiments, the additional therapeutic agents are selected from a different class of therapeutic agents. In some embodiments, the additional therapeutic agent is suitable for treating and/or preventing HIV infection. In some embodiments, the additional therapeutic agent is not for treating and/or preventing HIV infection.

In some embodiments, the combination therapy comprises administering a composition provided herein and one, two, three, four, or more additional therapeutic agents. In some embodiments, the combination therapy comprises administering a composition provided herein and two additional therapeutic agents. In some embodiments, the combination therapy comprises administering a composition provided herein and three additional therapeutic agents. In some embodiments, the combination therapy comprises administering a composition provided herein and four additional therapeutic agents.

In the some embodiments, the additional therapeutic agent is an anti-HIV agent. For example, in some embodiments, the additional therapeutic agent is selected from HIV protease inhibitors, HIV non-nucleoside or non-nucleotide inhibitors of reverse transcriptase, HIV nucleoside or nucleotide inhibitors of reverse transcriptase, HIV integrase inhibitors, HIV non-catalytic site (or allosteric) integrase inhibitors, HIV entry inhibitors, HIV maturation inhibitors, HIV capsid inhibitors, HIV Tat or Rev inhibitors, immunomodulators, immunotherapeutic agents, antibody-drug conjugates, gene modifiers, gene editors (such as CRISPR/Cas9, zinc finger nucleases, homing nucleases, synthetic nucleases, TALENs), cell therapies (such as chimeric antigen receptor T-cell, CAR-T, and engineered T-cell receptors, TCR-T, autologous T-cell therapies, engineered B cells), latency reversing agents, immune-based therapies, phosphatidylinositol 3-kinase (PI3K) inhibitors, HIV antibodies, bispecific antibodies and “antibody-like” therapeutic proteins, HIV p17 matrix protein inhibitors, IL-13 antagonists, peptidyl-prolyl cis-trans isomerase A modulators, protein disulfide isomerase inhibitors, complement C5a receptor antagonists, DNA methyltransferase inhibitor, HIV vif gene modulators, Vif dimerization antagonists, HIV-1 viral infectivity factor inhibitors, HIV-1 Nef modulators, Hck tyrosine kinase modulators, mixed lineage kinase-3 (MLK-3) inhibitors, HIV-1 splicing inhibitors, integrin antagonists, nucleoprotein inhibitors, splicing factor modulators, COMM domain containing protein 1 modulators, HIV ribonuclease H inhibitors, retrocyclin modulators, CDK-9 inhibitors, dendritic ICAM-3 grabbing nonintegrin 1 inhibitors, HIV GAG protein inhibitors, HIV POL protein inhibitors, Complement Factor H modulators, ubiquitin ligase inhibitors, deoxycytidine kinase inhibitors, cyclin dependent kinase inhibitors, proprotein convertase PC9 stimulators, ATP dependent RNA helicase DDX3X inhibitors, reverse transcriptase priming complex inhibitors, G6PD and NADH-oxidase inhibitors, pharmacokinetic enhancers, HIV gene therapy, HIV vaccines, and combinations thereof.

In some embodiments, the additional therapeutic agent is selected from the group consisting of combination drugs for treating and/or preventing HIV infection, other drugs for treating HIV, HIV protease inhibitors, HIV reverse transcriptase inhibitors, HIV integrase inhibitors, HIV non-catalytic site (or allosteric) integrase inhibitors, HIV entry (fusion) inhibitors, HIV maturation inhibitors, latency reversing agents, capsid inhibitors, immune-based therapies, PI3K inhibitors, HIV antibodies, and bispecific antibodies, and “antibody-like” therapeutic proteins, and combinations thereof.

In some embodiments, the additional therapeutic agent is a combination drug treating and/or preventing HIV infection. Examples of combination drugs for treating and/or preventing HIV infection include ATRIPLA® (efavirenz, tenofovir disoproxil fumarate, and emtricitabine); COMPLERA® (EVIPLERA®; rilpivirine, tenofovir disoproxil fumarate, and emtricitabine); STRIBILD® (elvitegravir, cobicistat, tenofovir disoproxil fumarate, and emtricitabine); TRUVADA® (tenofovir disoproxil fumarate and emtricitabine; TDF+FTC); DESCOVY® (tenofovir alafenamide and emtricitabine); ODEFSEY® (tenofovir alafenamide, emtricitabine, and rilpivirine); GENVOYA® (tenofovir alafenamide, emtricitabine, cobicistat, and elvitegravir); darunavir, tenofovir alafenamide hemifumarate, emtricitabine, and cobicistat; efavirenz, lamivudine, and tenofovir disoproxil fumarate; lamivudine and tenofovir disoproxil fumarate; tenofovir and lamivudine; tenofovir alafenamide and emtricitabine; tenofovir alafenamide hemifumarate and emtricitabine; tenofovir alafenamide hemifumarate, emtricitabine, and rilpivirine; tenofovir alafenamide hemifumarate, emtricitabine, cobicistat, and elvitegravir; COMBIVIR® (zidovudine and lamivudine; AZT+3TC); EPZICOM® (LIVEXA®; abacavir sulfate and lamivudine; ABC+3TC); KALETRA® (ALUVIA®; lopinavir and ritonavir); TRIUMEQ® (dolutegravir, abacavir, and lamivudine); BIKTARVY (bictegravir+emtricitabine+tenofovir alafenamide), DOVATO, TRIZIVIR® (abacavir sulfate, zidovudine, and lamivudine; ABC+AZT+3TC); atazanavir and cobicistat; atazanavir sulfate and cobicistat; atazanavir sulfate and ritonavir; darunavir and cobicistat; dolutegravir and rilpivirine; dolutegravir and rilpivirine hydrochloride; dolutegravir, abacavir sulfate, and lamivudine; lamivudine, nevirapine, and zidovudine; raltegravir and lamivudine; doravirine, lamivudine, and tenofovir disoproxil fumarate; doravirine, lamivudine, and tenofovir disoproxil; dolutegravir+lamivudine, lamivudine+abacavir+zidovudine, lamivudine+abacavir, lamivudine+tenofovir disoproxil fumarate, lamivudine+zidovudine+nevirapine, lopinavir+ritonavir, lopinavir+ritonavir+abacavir+lamivudine, lopinavir+ritonavir+zidovudine+lamivudine, tenofovir+lamivudine, and tenofovir disoproxil fumarate+emtricitabine+rilpivirine hydrochloride, lopinavir, ritonavir, zidovudine and lamivudine; cabotegravir+rilpivirine; elpida (elsulfavirine; VM-1500; VM-1500A).

In some embodiments, the additional therapeutic agent is a drug for treating and/or preventing HIV infection. Examples of other drugs for treating and/or preventing HIV infection include acemannan, alisporivir, BanLec, deferiprone, Gamimune, metenkefalin, naltrexone, Prolastin, REP 9, RPI-MN, VSSP, Hlviral, SB-728-T, 1,5-dicaffeoylquinic acid, rHIV7-shl-TAR-CCR5RZ, AAV-eCD4-Ig gene therapy, MazF gene therapy, BlockAide, ABX-464, AG-1105, APH-0812, BIT-225, CYT-107, HGTV-43, HPH-116, HS-10234, IMO-3100, IND-02, MK-1376, MK-2048, MK-4250, MK-8507, MK-8591, NOV-205, PA-1050040 (PA-040), PGN-007, SCY-635, SB-9200, SCB-719, TR-452, TEV-90110, TEV-90112, TEV-90111, TEV-90113, RN-18, Immuglo, and VIR-576.

In some embodiments, the additional therapeutic agent is a HIV protease inhibitor. Examples of HIV protease inhibitors include amprenavir, atazanavir, brecanavir, darunavir, fosamprenavir, fosamprenavir calcium, indinavir, indinavir sulfate, lopinavir, nelfinavir, nelfinavir mesylate, ritonavir, saquinavir, saquinavir mesylate, tipranavir, DG-17, TMB-657 (PPL-100), T-169, BL-008, MK-8122, TMB-607, and TMC-310911.

In some embodiments, the additional therapeutic agent is a reverse transcriptase inhibitor. Reverse transcriptase inhibitors can be non-nucleoside/non-nucleotide inhibitors or nucleoside/nucleotide inhibitors.

In some embodiments, the additional therapeutic agent is non-nucleoside or non-nucleotide reverse transcriptase inhibitors. Examples of HIV non-nucleoside or non-nucleotide inhibitors of reverse transcriptase include dapivirine, delavirdine, delavirdine mesylate, doravirine, efavirenz, etravirine, lentinan, nevirapine, rilpivirine, ACC-007, AIC-292, KM-023, PC-1005, and elsulfavirine (VM-1500).

In some embodiments, the additional therapeutic agent is a nucleoside or nucleotide reverse transcriptase inhibitor. Examples of nucleoside or nucleotide inhibitors of reverse transcriptase include adefovir, adefovir dipivoxil, azvudine, emtricitabine, tenofovir, tenofovir alafenamide, tenofovir alafenamide fumarate, tenofovir alafenamide hemifumarate, tenofovir disoproxil, tenofovir disoproxil fumarate, tenofovir disoproxil hemifumarate, VIDEX® and VIDEX EC® (didanosine, ddl), abacavir, abacavir sulfate, alovudine, apricitabine, censavudine, didanosine, elvucitabine, festinavir, fosalvudine tidoxil, CMX-157, dapivirine, doravirine, etravirine, OCR-5753, tenofovir disoproxil orotate, fozivudine tidoxil, lamivudine, phosphazid, stavudine, zalcitabine, zidovudine, rovafovir etalafenamide (GS-9131), GS-9148, MK-8504, MK-8591, MK-8583, VM-2500 and KP-1461.

In some embodiments, the additional therapeutic agent is a HIV integrase inhibitor. Examples of HIV integrase inhibitors include elvitegravir, curcumin, derivatives of curcumin, chicoric acid, derivatives of chicoric acid, 3,5-dicaffeoylquinic acid, derivatives of 3,5-dicaffeoylquinic acid, aurintricarboxylic acid, derivatives of aurintricarboxylic acid, caffeic acid phenethyl ester, derivatives of caffeic acid phenethyl ester, tyrphostin, derivatives of tyrphostin, quercetin, derivatives of quercetin, raltegravir, dolutegravir, JTK-351, bictegravir, AVX-15567, cabotegravir (long-acting injectable), diketo quinolin-4-1 derivatives, integrase-LEDGF inhibitor, ledgins, M-522, M-532, NSC-310217, NSC-371056, NSC-48240, NSC-642710, NSC-699171, NSC-699172, NSC-699173, NSC-699174, stilbenedisulfonic acid, T-169, VM-3500 and cabotegravir.

In some embodiments, a HIV integrase inhibitor is a non-catalytic site (i.e., allosteric) integrase inhibitor (NCINI). Examples of NCINIs include CX-05045, CX-05168, and CX-1442.

In some embodiments, the additional therapeutic agent is a HIV entry (fusion) inhibitor. Examples of HIV entry (fusion) inhibitors include cenicriviroc, CCR5 inhibitors, gp41 inhibitors, CD4 attachment inhibitors, gp120 inhibitors, and CXCR4 inhibitors.

In some embodiments, the additional therapeutic agent is a CCR5 inhibitor. Examples of CCR5 inhibitors include aplaviroc, vicriviroc, maraviroc, cenicriviroc, leronlimab (PRO-140), adaptavir (RAP-101), nifeviroc (TD-0232), anti-GP120/CD4 or CCR5 bispecific antibodies, B-07, MB-66, polypeptide C25P, TD-0680, and vMIP (Haimipu).

In some embodiments, the additional therapeutic agent is a gp41 inhibitor. Examples of gp41 inhibitors include albuvirtide, enfuvirtide, BMS-986197, enfuvirtide biobetter, enfuvirtide biosimilar, HIV-1 fusion inhibitors (P26-Bapc), ITV-1, ITV-2, ITV-3, ITV-4, PIE-12 trimer and sifuvirtide.

In some embodiments, the additional therapeutic agent is a CD4 attachment inhibitor. Examples of CD4 attachment inhibitors include ibalizumab and CADA analogs.

In some embodiments, the additional therapeutic agent is a gp120 inhibitor. Examples of gp120 inhibitors include Radha-108 (receptol) 3B3-PE38, BanLec, bentonite-based nanomedicine, fostemsavir tromethamine, IQP-0831, and BMS-663068.

In some embodiments, the additional therapeutic agent is a CXCR4 inhibitor. Examples of CXCR4 inhibitors include plerixafor, ALT-1188, N15 peptide, and vMIP (Haimipu).

In some embodiments, the additional therapeutic agent is a HIV maturation inhibitor. Examples of HIV maturation inhibitors include BMS-955176, GSK-3640254 and GSK-2838232.

In some embodiments, the additional therapeutic agent is a latency reversing agent. Examples of latency reversing agents include toll-like receptor (TLR) agonists (including TLR7 agonists, e.g., GS-9620), histone deacetylase (HDAC) inhibitors, proteasome inhibitors such as velcade, protein kinase C (PKC) activators, Smyd2 inhibitors, BET-bromodomain 4 (BRD4) inhibitors, ionomycin, IAP antagonists (inhibitor of apoptosis proteins, such as APG-1387, LBW-242), SMAC mimetics (including TL32711, LCL161, GDC-0917, HGS1029, AT-406), PMA, SAHA (suberanilohydroxamic acid, or suberoyl, anilide, and hydroxamic acid), NIZ-985, IL-15 modulating antibodies (including IL-15, IL-15 fusion proteins and IL-15 receptor agonists), JQ1, disulfiram, amphotericin B, and ubiquitin inhibitors such as largazole analogs, APH-0812, and GSK-343.

In some embodiments, the additional therapeutic agent is a HDAC (e.g., histone deacetylase 9 (HDAC9, HD7, HD7b, HD9, HDAC, HDAC7, HDAC7B, HDAC9B, HDAC9FL, HDRP, MITR; Gene ID: 9734) inhibitor. Examples of HDAC inhibitors include abexinostat, ACY-241, AR-42, BEBT-908, belinostat, CKD-581, CS-055 (HBI-8000), CUDC-907 (fimepinostat), entinostat, givinostat, mocetinostat, panobinostat, pracinostat, quisinostat (JNJ-26481585), resminostat, ricolinostat, romidepsin, SHP-141, valproic acid (VAL-001), vorinostat, tinostamustine, remetinostat, entinostat

In some embodiments, the additional therapeutic agent is a PKC activator. Examples of PKC activators include indolactam, prostratin, ingenol B, and DAG-lactones.

In some embodiments, the additional therapeutic agent is a capsid inhibitor. Examples of capsid inhibitors include capsid polymerization inhibitors or capsid disrupting compounds, HIV nucleocapsid p7 (NCp7) inhibitors such as azodicarbonamide, HIV p24 capsid protein inhibitors, GS-6207, GS-CA1, AVI-621, AVI-101, AVI-201, AVI-301, and AVI-CAN1-15 series, and compounds described in this patent (GSK WO2019/087016).

In some embodiments, the additional therapeutic agent is selected from one or more blockers or inhibitors of inhibitory immune checkpoint proteins or receptors and/or with one or more stimulators, activators or agonists of one or more stimulatory immune checkpoint proteins or receptors. Blockade or inhibition of inhibitory immune checkpoints can positively regulate T-cell or NK cell activation and prevent immune escape of infected cells. Activation or stimulation of stimulatory immune checkpoints can augment the effect of immune checkpoint inhibitors in infective therapeutics. In some embodiments, the immune checkpoint proteins or receptors regulate T cell responses (e.g., reviewed in Xu, et al., J Exp Clin Cancer Res. (2018) 37:110). In various embodiments, the immune checkpoint proteins or receptors regulate NK cell responses (e.g., reviewed in Davis, et al., Semin Immunol. (2017) 31:64-75 and Chiossone, et al., Nat Rev Immunol. (2018) 18(11):671-688)

Examples of immune checkpoint proteins or receptors include without limitation CD27, CD70; CD40, CD40LG; CD47, CD48 (SLAMF2), transmembrane and immunoglobulin domain containing 2 (TMIGD2, CD28H), CD84 (LY9B, SLAMF5), CD96, CD160, MS4A1 (CD20), CD244 (SLAMF4); CD276 (B7H3); V-set domain containing T cell activation inhibitor 1 (VTCN1, B7H4); V-set immunoregulatory receptor (VSIR, B7H5, VISTA); immunoglobulin superfamily member 11 (IGSF11, VSIG3); natural killer cell cytotoxicity receptor 3 ligand 1 (NCR3LG1, B7H6); HERV-H LTR-associating 2 (HHLA2, B7H7); inducible T cell co-stimulator (ICOS, CD278); inducible T cell costimulator ligand (ICOSLG, B7H2); TNF receptor superfamily member 4 (TNFRSF4, OX40); TNF superfamily member 4 (TNFSF4, OX40L); TNFRSF8 (CD30), TNFSF8 (CD30L); TNFRSF10A (CD261, DR4, TRAILR1), TNFRSF9 (CD137), TNFSF9 (CD137L); TNFRSF10B (CD262, DR5, TRAILR2), TNFRSF10 (TRAIL); TNFRSF14 (HVEM, CD270), TNFSF14 (HVEML); CD272 (B and T lymphocyte associated (BTLA)); TNFRSF17 (BCMA, CD269), TNFSF13B (BAFF); TNFRSF18 (GITR), TNFSF18 (GITRL); MHC class I polypeptide-related sequence A (MICA); MHC class I polypeptide-related sequence B (MICB); CD274 (CD274, PDL1, PD-L1); programmed cell death 1 (PDCD1, PD1, PD-1); cytotoxic T-lymphocyte associated protein 4 (CTLA4, CD152); CD80 (B7-1), CD28; nectin cell adhesion molecule 2 (NECTIN2, CD112); CD226 (DNAM-1); Poliovirus receptor (PVR) cell adhesion molecule (PVR, CD155); PVR related immunoglobulin domain containing (PVRIG, CD112R); T cell immunoreceptor with Ig and ITIM domains (TIGIT); T cell immunoglobulin and mucin domain containing 4 (TIMD4; TIM4); hepatitis A virus cellular receptor 2 (HAVCR2, TIMD3, TIM3); galectin 9 (LGALS9); lymphocyte activating 3 (LAG3, CD223); signaling lymphocytic activation molecule family member 1 (SLAMF1, SLAM, CD150); lymphocyte antigen 9 (LY9, CD229, SLAMF3); SLAM family member 6 (SLAMF6, CD352); SLAM family member 7 (SLAMF7, CD319); UL16 binding protein 1 (ULBP1); UL16 binding protein 2 (ULBP2); UL16 binding protein 3 (ULBP3); retinoic acid early transcript 1E (RAET1E; ULBP4); retinoic acid early transcript 1G (RAET1G; ULBP5); retinoic acid early transcript 1L (RAET1L; ULBP6); lymphocyte activating 3 (CD223); killer cell immunoglobulin like receptor, three Ig domains and long cytoplasmic tail 1 (KIR, CD158E1); killer cell lectin like receptor C1 (KLRC1, NKG2A, CD159A); killer cell lectin like receptor K1 (KLRK1, NKG2D, CD314); killer cell lectin like receptor C2 (KLRC2, CD159c, NKG2C); killer cell lectin like receptor C3 (KLRC3, NKG2E); killer cell lectin like receptor C4 (KLRC4, NKG2F); killer cell immunoglobulin like receptor, two Ig domains and long cytoplasmic tail 1 (KIR2DL1); killer cell immunoglobulin like receptor, two Ig domains and long cytoplasmic tail 2 (KIR2DL2); killer cell immunoglobulin like receptor, two Ig domains and long cytoplasmic tail 3 (KIR2DL3); killer cell immunoglobulin like receptor, three Ig domains and long cytoplasmic tail 1 (KIR3DL1); killer cell lectin like receptor D1 (KLRD1); and SLAM family member 7 (SLAMF7).

In some embodiments, the additional therapeutic agent is a blocker or inhibitor of one or more T-cell inhibitory immune checkpoint proteins or receptors. Examples of T-cell inhibitory immune checkpoint proteins or receptors include without limitation CD274 (CD274, PDL1, PD-L1); programmed cell death 1 ligand 2 (PDCD1LG2, PD-L2, CD273); programmed cell death 1 (PDCD1, PD1, PD-1); cytotoxic T-lymphocyte associated protein 4 (CTLA4, CD152); CD276 (B7H3); V-set domain containing T cell activation inhibitor 1 (VTCN1, B7H4); V-set immunoregulatory receptor (VSIR, B7H5, VISTA); immunoglobulin superfamily member 11 (IGSF11, VSIG3); TNFRSF14 (HVEM, CD270), TNFSF14 (HVEML); CD272 (B and T lymphocyte associated (BTLA)); PVR related immunoglobulin domain containing (PVRIG, CD112R); T cell immunoreceptor with Ig and ITIM domains (TIGIT); lymphocyte activating 3 (LAG3, CD223); hepatitis A virus cellular receptor 2 (HAVCR2, TIMD3, TIM3); galectin 9 (LGALS9); killer cell immunoglobulin like receptor, three Ig domains and long cytoplasmic tail 1 (KIR, CD158E1); killer cell immunoglobulin like receptor, two Ig domains and long cytoplasmic tail 1 (KIR2DL1); killer cell immunoglobulin like receptor, two Ig domains and long cytoplasmic tail 2 (KIR2DL2); killer cell immunoglobulin like receptor, two Ig domains and long cytoplasmic tail 3 (KIR2DL3); and killer cell immunoglobulin like receptor, three Ig domains and long cytoplasmic tail 1 (KIR3DL1). In various embodiments, the agents, as described herein, are combined with one or more agonist or activators of one or more T-cell stimulatory immune checkpoint proteins or receptors. Illustrative T-cell stimulatory immune checkpoint proteins or receptors include without limitation CD27, CD70; CD40, CD40LG; inducible T cell costimulator (ICOS, CD278); inducible T cell costimulator ligand (ICOSLG, B7H2); TNF receptor superfamily member 4 (TNFRSF4, OX40); TNF superfamily member 4 (TNFSF4, OX40L); TNFRSF9 (CD137), TNFSF9 (CD137L); TNFRSF18 (GITR), TNF SF18 (GITRL); CD80 (B7-1), CD28; nectin cell adhesion molecule 2 (NECTIN2, CD112); CD226 (DNAM-1); CD244 (2B4, SLAMF4), Poliovirus receptor (PVR) cell adhesion molecule (PVR, CD155). See, e.g., Xu, et al., J Exp Clin Cancer Res. (2018) 37:110.

In some embodiments, the additional therapeutic agent is a blocker or inhibitor of one or more NK-cell inhibitory immune checkpoint proteins or receptors. Examples of NK-cell inhibitory immune checkpoint proteins or receptors include without limitation killer cell immunoglobulin like receptor, three Ig domains and long cytoplasmic tail 1 (KIR, CD158E1); killer cell immunoglobulin like receptor, two Ig domains and long cytoplasmic tail 1 (KIR2DL1); killer cell immunoglobulin like receptor, two Ig domains and long cytoplasmic tail 2 (KIR2DL2); killer cell immunoglobulin like receptor, two Ig domains and long cytoplasmic tail 3 (KIR2DL3); killer cell immunoglobulin like receptor, three Ig domains and long cytoplasmic tail 1 (KIR3DL1); killer cell lectin like receptor C1 (KLRC1, NKG2A, CD159A); and killer cell lectin like receptor D1 (KLRD1, CD94). In various embodiments, the agents as described herein, are combined with one or more agonist or activators of one or more NK-cell stimulatory immune checkpoint proteins or receptors. Illustrative NK-cell stimulatory immune checkpoint proteins or receptors include without limitation CD16, CD226 (DNAM-1); CD244 (2B4, SLAMF4); killer cell lectin like receptor K1 (KLRK1, NKG2D, CD314); SLAM family member 7 (SLAMF7). See, e.g., Davis, et al., Semin Immunol. (2017) 31:64-75; Fang, et al., Semin Immunol. (2017) 31:37-54; and Chiossone, et al., Nat Rev Immunol. (2018) 18(11):671-688.

In some embodiments, the one or more immune checkpoint inhibitors comprises a proteinaceous (e.g., antibody or fragment thereof, or antibody mimetic) inhibitor of PD-L1 (CD274), PD-1 (PDCD1) or CTLA4. In some embodiments, the one or more immune checkpoint inhibitors comprises a small organic molecule inhibitor of PD-L1 (CD274), PD-1 (PDCD1) or CTLA4. In some embodiments, the small molecule inhibitor of CD274 or PDCD1 is selected from the group consisting of GS-4224, GS-4416, INCB086550 and MAX10181. In some embodiments, the small molecule inhibitor of CTLA4 comprises BPI-002.

In some embodiments, the additional therapeutic agent is an inhibitor of CLTA4. Examples of inhibitors of CLTA4 include ipilimumab, tremelimumab, BMS-986218, AGEN1181, AGEN1884, BMS-986249, MK-1308, REGN-4659, ADU-1604, CS-1002, BCD-145, APL-509, JS-007, BA-3071, ONC-392, AGEN-2041, JHL-1155, KN-044, CG-0161, ATOR-1144, PBI-5D3H5, BPI-002, as well as multi-specific inhibitors FPT-155 (CTLA4/PD-L1/CD28), PF-06936308 (PD-1/CTLA4), MGD-019 (PD-1/CTLA4), KN-046 (PD-1/CTLA4), MEDI-5752 (CTLA4/PD-1), XmAb-20717 (PD-1/CTLA4), and AK-104 (CTLA4/PD-1).

In some embodiments, the additional therapeutic agent is an inhibitors of PD-L1 (CD274) or PD-1 (PDCD1). Examples of inhibitors of PD-L1 (CD274) or PD-1 (PDCD1) include pembrolizumab, nivolumab, cemiplimab, pidilizumab, AMP-224, MEDI0680 (AMP-514), spartalizumab, atezolizumab, avelumab, durvalumab, BMS-936559, CK-301, PF-06801591, BGB-A317 (tislelizumab), GLS-010 (WBP-3055), AK-103 (HX-008), AK-105, CS-1003, HLX-10, MGA-012, BI-754091, AGEN-2034, JS-001 (toripalimab), JNJ-63723283, genolimzumab (CBT-501), LZM-009, BCD-100, LY-3300054, SHR-1201, SHR-1210 (camrelizumab), Sym-021, ABBV-181, PD1-PIK, BAT-1306, (MSB0010718C), CX-072, CBT-502, TSR-042 (dostarlimab), MSB-2311, JTX-4014, BGB-A333, SHR-1316, CS-1001 (WBP-3155, KN-035, IBI-308 (sintilimab), HLX-20, KL-A167, STI-A1014, STI-A1015 (IMC-001), BCD-135, FAZ-053, TQB-2450, MDX1105-01, GS-4224, GS-4416, INCB086550, MAX10181, as well as multi-specific inhibitors FPT-155 (CTLA4/PD-L1/CD28), PF-06936308 (PD-1/CTLA4), MGD-013 (PD-1/LAG-3), FS-118 (LAG-3/PD-L1) MGD-019 (PD-1/CTLA4), KN-046 (PD-1/CTLA4), MEDI-5752 (CTLA4/PD-1), RO-7121661 (PD-1/TIM-3), XmAb-20717 (PD-1/CTLA4), AK-104 (CTLA4/PD-1), M7824 (PD-L1/TGFβ-EC domain), CA-170 (PD-L1/VISTA), CDX-527 (CD27/PD-L1), LY-3415244 (TIM3/PDL1), and INBRX-105 (4-1BB/PDL1).

In some embodiments, the additional therapeutic agent is an anti-TIGIT antibody. Examples of anti-TIGIT antibodies include BMS-986207, RG-6058, and AGEN-1307.

In some embodiments, the additional therapeutic agent is an agonist of one or more TNF receptor superfamily (TNFRSF) members, e.g., an agonist of one or more of TNFRSF1A (NCBI Gene ID: 7132), TNFRSF1B (NCBI Gene ID: 7133), TNFRSF4 (OX40, CD134; NCBI Gene ID: 7293), TNFRSF5 (CD40; NCBI Gene ID: 958), TNFRSF6 (FAS, NCBI Gene ID: 355), TNFRSF7 (CD27, NCBI Gene ID: 939), TNFRSF8 (CD30, NCBI Gene ID: 943), TNFRSF9 (4-1BB, CD137, NCBI Gene ID: 3604), TNFRSF10A (CD261, DR4, TRAILR1, NCBI Gene ID: 8797), TNFRSF10B (CD262, DR5, TRAILR2, NCBI Gene ID: 8795), TNFRSF10C (CD263, TRAILR3, NCBI Gene ID: 8794), TNFRSF10D (CD264, TRAILR4, NCBI Gene ID: 8793), TNFRSF11A (CD265, RANK, NCBI Gene ID: 8792), TNFRSF11B (NCBI Gene ID: 4982), TNFRSF12A (CD266, NCBI Gene ID: 51330), TNFRSF13B (CD267, NCBI Gene ID: 23495), TNFRSF13C (CD268, NCBI Gene ID: 115650), TNFRSF16 (NGFR, CD271, NCBI Gene ID: 4804), TNFRSF17 (BCMA, CD269, NCBI Gene ID: 608), TNFRSF18 (GITR, CD357, NCBI Gene ID: 8784), TNFRSF19 (NCBI Gene ID: 55504), TNFRSF21 (CD358, DR6, NCBI Gene ID: 27242), and TNFRSF25 (DR3, NCBI Gene ID: 8718).

In some embodiments, the additional therapeutic agent is an anti-TNFRSF4 (OX40) antibody. Examples of anti-TNFRSF4 (OX40) antibodies include MEDI6469, MEDI6383, MEDI0562 (tavolixizumab), MOXR0916, PF-04518600, RG-7888, GSK-3174998, INCAGN1949, BMS-986178, GBR-8383, ABBV-368, and those described in WO2016179517, WO2017096179, WO2017096182, WO2017096281, and WO2018089628.

In some embodiments, the additional therapeutic agent is an anti-TNFRSF5 (CD40) antibody. Examples of anti-TNFRSF5 (CD40) antibodies include RG7876, SEA-CD40, APX-005M and ABBV-428.

In some embodiments, the additional therapeutic agent is an anti-TNFRSF7 (CD27) antibody. An example of an anti-TNFRSF7 (CD27) antibody is varlilumab (CDX-1127).

In some embodiments, the additional therapeutic agent is an anti-TNFRSF9 (4-1BB, CD137) antibody. Examples of anti-TNFRSF9 (4-1BB, CD137) antibodies include urelumab, utomilumab (PF-05082566), AGEN2373 and ADG-106.

In some embodiments, the additional therapeutic agent is an anti-TNFRSF18 (GITR) antibody. Examples of anti-TNFRSF18 (GITR) antibodies include MEDI1873, FPA-154, INCAGN-1876, TRX-518, BMS-986156, MK-1248, GWN-323, and those described in WO2017096179, WO2017096276, WO2017096189, and WO2018089628.

In some embodiments, the additional therapeutic agent is an antibody, or fragment thereof, co-targeting TNFRSF4 (OX40) and TNFRSF18 (GITR). Such antibodies are described, e.g., in WO2017096179 and WO2018089628.

In some embodiments, the additional therapeutic agent is a bi-specific NK-cell engager (BiKE) or a tri-specific NK-cell engager (TriKE) (e.g., not having an Fc) or bi-specific antibody (e.g., having an Fc) against an NK cell activating receptor, e.g., CD16A, C-type lectin receptors (CD94/NKG2C, NKG2D, NKG2E/H and NKG2F), natural cytotoxicity receptors (NKp30, NKp44 and NKp46), killer cell C-type lectin-like receptor (NKp65, NKp80), Fc receptor FcγR (which mediates antibody-dependent cell cytotoxicity), SLAM family receptors (e.g., 2B4, SLAM6 and SLAM7), killer cell immunoglobulin-like receptors (KIR) (KIR-2DS and KIR-3DS), DNAM-1 and CD137 (41BB). As appropriate, the anti-CD16 binding bi-specific molecules may or may not have an Fc. Illustrative bi-specific NK-cell engagers include those that target CD16 and one or more HIV-associated antigens. BiKEs and TriKEs are described, e.g., in Felices, et al., Methods Mol Biol. (2016) 1441:333-346; Fang, et al., Semin Immunol. (2017) 31:37-54. Examples of a trispecific NK cell engager (TRiKE) include OXS-3550, and CD16-IL-15-B7H3 TriKe.

In some embodiments, the additional therapeutic agent is an inhibitor of indoleamine 2,3-dioxygenase 1 (IDO1; NCBI Gene ID: 3620). Examples of IDO1 inhibitors include BLV-0801, epacadostat, F-001287, GBV-1012, GBV-1028, GDC-0919, indoximod, NKTR-218, NLG-919-based vaccine, PF-06840003, pyranonaphthoquinone derivatives (SN-35837), resminostat, SBLK-200802, BMS-986205, and shIDO-ST, EOS-200271, KHK-2455, LY-3381916.

In some embodiments, the additional therapeutic agent is an agonist of a toll-like receptor (TLR), e.g., an agonist of TLR1 (NCBI Gene ID: 7096), TLR2 (NCBI Gene ID: 7097), TLR3 (NCBI Gene ID: 7098), TLR4 (NCBI Gene ID: 7099), TLR5 (NCBI Gene ID: 7100), TLR6 (NCBI Gene ID: 10333), TLR7 (NCBI Gene ID: 51284), TLR8 (NCBI Gene ID: 51311), TLR9 (NCBI Gene ID: 54106), and/or TLR10 (NCBI Gene ID: 81793). Example TLR7 agonists include AL-034, DSP-0509, GS-9620 (vesatolimod), LHC-165, TMX-101 (imiquimod), GSK-2245035, resiquimod, DSR-6434, DSP-3025, IMO-4200, MCT-465, MEDI-9197, 3M-051, SB-9922, 3M-052, Limtop, TMX-30X, TMX-202, RG-7863, RG-7854, RG-7795, and the compounds disclosed in US20100143301 (Gilead Sciences), US20110098248 (Gilead Sciences), and US20090047249 (Gilead Sciences), US20140045849 (Janssen), US20140073642 (Janssen), WO2014/056953 (Janssen), WO2014/076221 (Janssen), WO2014/128189 (Janssen), US20140350031 (Janssen), WO2014/023813 (Janssen), US20080234251 (Array Biopharma), US20080306050 (Array Biopharma), US20100029585 (Ventirx Pharma), US20110092485 (Ventirx Pharma), US20110118235 (Ventirx Pharma), US20120082658 (Ventirx Pharma), US20120219615 (Ventirx Pharma), US20140066432 (Ventirx Pharma), US20140088085 (Ventirx Pharma), US20140275167 (Novira Therapeutics), and US20130251673 (Novira Therapeutics). An example of a TLR7/TLR8 agonist is NKTR-262, telratolimod and BDB-001. Examples of TLR8 agonists include E-6887, IMO-4200, IMO-8400, IMO-9200, MCT-465, MEDI-9197, motolimod, resiquimod, GS-9688, VTX-1463, VTX-763, 3M-051, 3M-052, and the compounds disclosed in US20140045849 (Janssen), US20140073642 (Janssen), WO2014/056953 (Janssen), WO2014/076221 (Janssen), WO2014/128189 (Janssen), US20140350031 (Janssen), WO2014/023813 (Janssen), US20080234251 (Array Biopharma), US20080306050 (Array Biopharma), US20100029585 (Ventirx Pharma), US20110092485 (Ventirx Pharma), US20110118235 (Ventirx Pharma), US20120082658 (Ventirx Pharma), US20120219615 (Ventirx Pharma), US20140066432 (Ventirx Pharma), US20140088085 (Ventirx Pharma), US20140275167 (Novira Therapeutics), and US20130251673 (Novira Therapeutics). Example TLR9 agonists include AST-008, cobitolimod, CMP-001, IMO-2055, IMO-2125, litenimod, MGN-1601, BB-001, BB-006, IMO-3100, IMO-8400, IR-103, IMO-9200, agatolimod, DIMS-9054, DV-1079, DV-1179, AZD-1419, lefitolimod (MGN-1703), CYT-003, CYT-003-QbG10, tilsotolimod and PUL-042. Examples of TLR3 agonists include rintatolimod, poly-ICLC, RIBOXXON®, Apoxxim, RIBOXXIM®, IPH-33, MCT-465, MCT-475, and ND-1.1. Examples of TLR4 agonists include G-100, and GSK-1795091.

In some embodiments, the additional therapeutic agent is a stimulator of interferon genes (STING) agonist or activator. Examples of STING receptor agonists or activators include ADU-S100 (MIW-815), SB-11285, MK-1454, SR-8291, AdVCA0848, GSK-532, SYN-STING, MSA-1, SR-8291, 5,6-dimethylxanthenone-4-acetic acid (DMXAA), cyclic-GAMP (cGAMP) and cyclic-di-AMP.

In some embodiments, the additional therapeutic agent is a RIG-I modulator such as RGT-100, or a NOD2 modulator, such as SB-9200, and IR-103.

In some embodiments, the additional therapeutic agent is an anti-TIM-3 antibody, such as TSR-022, LY-3321367, MBG-453, INCAGN-2390.

In some embodiments, the additional therapeutic agent is an anti LAG-3 (Lymphocyte-activation) antibody, such as relatlimab (ONO-4482), LAG-525, MK-4280, REGN-3767, INCAGN2385.

In some embodiments, the additional therapeutic agent is an interleukin agonist, such as IL-2, IL-7, IL-15, IL-10, IL-12 agonists; examples of IL-2 agonists such as proleukin (aldesleukin, IL-2); pegylated IL-2 (eg NKTR-214); modified variants of IL-2 (eg THOR-707), bempegaldesleukin, AIC-284, ALKS-4230, CUI-101, Neo-2/15; examples of IL-15 agonists, such as ALT-803, NKTR-255, and hetIL-15, interleukin-15/Fc fusion protein, AM-0015, NIZ-985, SO-C101, IL-15 Synthorin (pegylated Il-15), P-22339, and a IL-15-PD-1 fusion protein N-809; examples of IL-7 include CYT-107.

In some embodiments, the additional therapeutic agent is an immune-based therapy selected from include interferon alfa; interferon alfa-2b; interferon alfa-n3; pegylated interferon alfa; interferon gamma; Flt3 agonists; gepon; normferon, peginterferon alfa-2a, peginterferon alfa-2b, and RPI-MN.

In some embodiments, the additional therapeutic agent is a phosphatidylinositol 3-kinase (PI3K) inhibitor. Examples of PI3K inhibitors include idelalisib, alpelisib, buparlisib, CAI orotate, copanlisib, duvelisib, gedatolisib, neratinib, panulisib, perifosine, pictilisib, pilaralisib, puquitinib mesylate, rigosertib, rigosertib sodium, sonolisib, taselisib, AMG-319, AZD-8186, BAY-1082439, CLR-1401, CLR-457, CUDC-907, DS-7423, EN-3342, GSK-2126458, GSK-2269577, GSK-2636771, INCB-040093, LY-3023414, MLN-1117, PQR-309, RG-7666, RP-6530, RV-1729, SAR-245409, SAR-260301, SF-1126, TGR-1202, UCB-5857, VS-5584, XL-765, and ZSTK-474.

In some embodiments, the additional therapeutic agent is an integrin alpha-4/beta-7 antagonist. Examples of integrin alpha-4/beta-7 antagonists include PTG-100, TRK-170, abrilumab, etrolizumab, carotegrast methyl, and vedolizumab.

In some embodiments, the additional therapeutic agent is a HIV antibody, bispecific antibody, or “antibody-like” therapeutic protein. Examples of HIV antibodies, bispecific antibodies, and “antibody-like” therapeutic proteins include DARTs®, DUOBODIES®, BITES®, XmAbs®, TandAbs®, Fab derivatives, bNAbs (broadly neutralizing HIV-1 antibodies), TMB-360, and those targeting HIV gp120 or gp41, antibody-Recruiting Molecules targeting HIV, anti-CD63 monoclonal antibodies, anti-GB virus C antibodies, anti-GP120/CD4, CCR5 bispecific antibodies, anti-Nef single domain antibodies, anti-Rev antibody, camelid derived anti-CD18 antibodies, camelid-derived anti-ICAM-1 antibodies, DCVax-001, gp140 targeted antibodies, gp41-based HIV therapeutic antibodies, human recombinant mAbs (PGT-121), ibalizumab, Immuglo, and MB-66.

In some embodiments, the additional therapeutic agent is a bNAbs. Examples include those described in U.S. Pat. Nos. 8,673,307, 9,493,549, 9,783,594, WO2014/063059, WO2012/158948, WO2015/117008, and PCT/US2015/41272, and WO2017/096221, including antibodies 12A12, 12A21, NIH45-46, bANC131, 8ANC134, IB2530, INC9, 8ANC195. 8ANC196, 10-259, 10-303, 10-410, 10-847, 10-996, 10-1074, 10-1121, 10-1130, 10-1146, 10-1341, 10-1369, and 10-1074GM. Additional examples include those described in Klein et al., Nature, 492(7427): 118-22 (2012), Horwitz et al., Proc Natl Acad Sci USA, 110(41): 16538-43 (2013), Scheid, et al., Science, 333: 1633-1637 (2011), Scheid, et al., Nature, 458:636-640 (2009), Eroshkin et al, Nucleic Acids Res., 42 (Database issue):D1 133-9 (2014), Mascola et al., Immunol Rev., 254(0:225-44 (2013), such as 2F5, 4E10, M66.6, CAP206-CH12, 10E81 (all of which bind the MPER of gp41); PG9, PG16, CH01-04 (all of which bind V1V2-glycan), 2G12 (which binds to outer domain glycan); b12, HJ16, CH103-106, VRC01-03, VRC-PG04, 04b, VRC-CH30-34, 3BNC62, 3BNC89, 3BNC91, 3BNC95, 3BNC104, 3BNC176, and 8ANC131 (all of which bind to the CD4 binding site).

In some embodiments, the additional therapeutic agent is a broadly neutralizing antibody, such as those described in e.g., U.S. Pat. Nos. 8,673,307; 9,493,549; 9,783,594; and WO 2012/154312; WO2012/158948; WO 2013/086533; WO 2013/142324; WO2014/063059; WO 2014/089152, WO 2015/048462; WO 2015/103549; WO 2015/117008; WO2016/014484; WO 2016/154003; WO 2016/196975; WO 2016/149710; WO2017/096221; WO 2017/133639; WO 2017/133640, which are hereby incorporated herein by reference in their entireties for all purposes. Additional examples include those described in Sajadi, et al., Cell. (2018) 173(7):1783-1795; Sajadi, et al., J Infect Dis. (2016) 213(1):156-64; Klein et al., Nature, 492(7427): 118-22 (2012), Horwitz et al., Proc Natl Acad Sci USA, 110(41): 16538-43 (2013), Scheid, et al., Science, 333: 1633-1637 (2011), Scheid, et al., Nature, 458:636-640 (2009), Eroshkin et al, Nucleic Acids Res., 42 (Database issue):D1 133-9 (2014), Mascola et al., Immunol Rev., 254(0:225-44 (2013), such as 2F5, 4E10, M66.6, CAP206-CH12, 10E8, 10E8v4, 10E8-5R-100cF, DH511.11P, 7b2, and LN01 (all of which bind the MPER of gp41).

Additional antibodies that can be used as the additional therapeutic agent include bavituximab, UB-421, BF520.1, CH01, CH59, C2F5, C4E10, C2F5+C2G12+C4E10, 3BNC117, 3BNC117-LS, 3BNC60, DH270.1, DH270.6, D1D2, 10-1074-LS, GS-9722, DH411-2, BG18, PGT145, PGT121, PGT-121.60, PGT-121.66, PGT122, PGT-123, PGT-124, PGT-125, PGT-126, PGT-151, PGT-130, PGT-133, PGT-134, PGT-135, PGT-128, PGT-136, PGT-137, PGT-138, PGT-139, MDX010 (ipilimumab), DH511, DH511-2, N6, N6LS, N49P6, N49P7, N49P7.1, N49P9, N49P11, N60P1.1, N60P25.1, N60P2.1, N60P31.1, N60P22, NIH 45-46, PGC14, PGG14, PGT-142, PGT-143, PGT-144, PGDM1400, PGDM12, PGDM21, PCDN-33A, 2Dm2m, 4Dm2m, 6Dm2m, PGDM1400, MDX010 (ipilimumab), VRC01, VRC-01-LS, A32, 7B2, 10E8, VRC-07-523, VRC07-523LS, VRC24, VRC41.01, 10E8VLS, 3810109, 10E8v4, IMC-HIV, iMabm36, eCD4-Ig, IOMA, CAP256-VRC26.25, DRVIA7, VRC-HIVMAB080-00-AB, VRC-HIVMAB060-00-AB, P2G12, VRC07, 354BG8, 354BG18, 354BG42, 354BG33, 354BG129, 354BG188, 354BG411, 354BG426, VRC29.03, CAP256, CAP256-VRC26.08, CAP256-VRC26.09, CAP256-VRC26.25, PCT64-24E and VRC38.01, PGT-151, CAP248-2B, 35022, ACS202, VRC34 and VRC34.01, 10E8, 10E8v4, 10E8-5R-100cF, 4E10, DH511.11P, 2F5, 7b2, and LN01.

Examples of HIV bispecific and trispecific antibodies include MGD014, B12BiTe, TMB-bispecific, SAR-441236, VRC-01/PGDM-1400/10E8v4, 10E8.4/iMab, 10E8v4/PGT121-VRC01.

Examples of in vivo delivered bNAbs include AAV8-VRC07; mRNA encoding anti-HIV antibody VRC01; and engineered B-cells encoding 3BNC117 (Hartweger et al, J. Exp. Med. 2019, 1301).

In some embodiments, the additional therapeutic agent is a pharmacokinetic enhancer. Examples pharmacokinetic enhancers include cobicistat and ritonavir.

Examples of additional therapeutic agents include the compounds disclosed in WO 2004/096286 (Gilead Sciences), WO 2006/015261 (Gilead Sciences), WO 2006/110157 (Gilead Sciences), WO 2012/003497 (Gilead Sciences), WO 2012/003498 (Gilead Sciences), WO 2012/145728 (Gilead Sciences), WO 2013/006738 (Gilead Sciences), WO 2013/159064 (Gilead Sciences), WO 2014/100323 (Gilead Sciences), US 2013/0165489 (University of Pennsylvania), US 2014/0221378 (Japan Tobacco), US 2014/0221380 (Japan Tobacco), WO 2009/062285 (Boehringer Ingelheim), WO 2010/130034 (Boehringer Ingelheim), WO 2013/006792 (Pharma Resources), US 20140221356 (Gilead Sciences), US 20100143301 (Gilead Sciences) and WO 2013/091096 (Boehringer Ingelheim).

In some embodiments, the additional therapeutic agent is a HIV vaccine. Examples of HIV vaccines include peptide vaccines, recombinant subunit protein vaccines, live vector vaccines, DNA vaccines, CD4-derived peptide vaccines, vaccine combinations, adenoviral vector vaccines (an adenoviral vector such as Ad5, Ad26 or Ad35), simian adenovirus (chimpanzee, gorilla, rhesus i.e. rhAd), adeno-associated virus vector vaccines, Chimpanzee adenoviral vaccines (e.g., ChAdOX1, ChAd68, ChAd3, ChAd63, ChAd83, ChAd155, ChAd157, Pan5, Pan6, Pan7, Pan9), Coxsackieviruses based vaccines, enteric virus based vaccines, Gorilla adenovirus vaccines, lentiviral vector based vaccine, arenavirus vaccines (such as LCMV, Pichinde), bi-segmented or tri-segmented arenavirus based vaccine, measles virus based vaccine, flavivirus vector based vaccines, tobacco mosaic virus vector based vaccine, Varicella-zoster virus based vaccine, Human parainfluenza virus 3 (PIV3) based vaccines, poxvirus based vaccine (modified vaccinia virus Ankara (MVA), orthopoxvirus-derived NYVAC, and avipoxvirus-derived ALVAC (canarypox virus) strains); fowlpox virus based vaccine, rhabdovirus-based vaccines, such as VSV and marabavirus; recombinant human CMV (rhCMV) based vaccine, alphavirus-based vaccines, such as semliki forest virus, venezuelan equine encephalitis virus and sindbis virus; (see Lauer, Clinical and Vaccine Immunology, 2017, DOI: 10.1128/CVI.00298-16); LNP formulated mRNA based therapeutic vaccines; LNP-formulated self-replicating RNA/self-amplifying RNA vaccines.

Examples of vaccines include: rgp120 (AIDSVAX), ALVAC HIV (vCP1521)/AIDSVAX B/E (gp120) (RV144), monomeric gp120 HIV-1 subtype C vaccine, Remune, ITV-1, Contre Vir, Ad5-ENVA-48, DCVax-001 (CDX-2401), Vacc-4x, Vacc-05, VAC-3 S, multiclade DNA recombinant adenovirus-5 (rAd5), rAd5 gag-pol env A/B/C vaccine, Pennvax-G, Pennvax-GP, Pennvax-G/MVA-CMDR, HIV-TriMix-mRNA vaccine, HIV-LAMP-vax, Ad35, Ad35-GRIN, NAcGM3/VSSP ISA-51, poly-ICLC adjuvanted vaccines, TatImmune, GTU-multiHIV (FIT-06), gp140[delta]V2.TV1+MF-59, rVSVIN HIV-1 gag vaccine, SeV-Gag vaccine, AT-20, DNK-4, ad35-Grin/ENV, TBC-M4, HIVAX, HIVAX-2, NYVAC-HIV-PT1, NYVAC-HIV-PT4, DNA-HIV-PT123, rAAV1-PG9DP, GOVX-B11, GOVX-B21, TVI-HIV-1, Ad-4 (Ad4-env Clade C+Ad4-mGag), Paxvax, EN41-UGR7C, EN41-FPA2, PreVaxTat, AE-H, MYM-V101, CombiHlVvac, ADVAX, MYM-V201, MVA-CMDR, DNA-Ad5 gag/pol/nef/nev (HVTN505), MVATG-17401, ETV-01, CDX-1401, rcAD26.MOS1.HIV-Env, Ad26.Mod.HIV vaccine, Ad26.Mod.HIV+MVA mosaic vaccine+gp140, AGS-004, AVX-101, AVX-201, PEP-6409, SAV-001, ThV-01, TL-01, TUTI-16, VGX-3300, IHV-001, and virus-like particle vaccines such as pseudovirion vaccine, CombiVICHvac, LFn-p24 B/C fusion vaccine, GTU-based DNA vaccine, HIV gag/pol/nef/env DNA vaccine, anti-TAT HIV vaccine, conjugate polypeptides vaccine, dendritic-cell vaccines (such as DermaVir), gag-based DNA vaccine, GI-2010, gp41 HIV-1 vaccine, HIV vaccine (PIKA adjuvant), i-key/MHC class II epitope hybrid peptide vaccines, ITV-2, ITV-3, ITV-4, LIPO-5, multiclade Env vaccine, MVA vaccine, Pennvax-GP, pp71-deficient HCMV vector HIV gag vaccine, rgp160 HIV vaccine, RNActive HIV vaccine, SCB-703, Tat Oyi vaccine, TBC-M4, UBI HIV gp120, Vacc-4x+romidepsin, variant gp120 polypeptide vaccine, rAd5 gag-pol env A/B/C vaccine, DNA.HTI and MVA.HTI, VRC-HIVDNA016-00-VP+VRC-HIVADV014-00-VP, INO-6145, JNJ-9220, gp145 C.6980; eOD-GT8 60mer based vaccine, PD-201401, env (A, B, C, A/E)/gag (C) DNA Vaccine, gp120 (A, B, C, A/E) protein vaccine, PDPHV-201401, Ad4-EnvCN54, EnvSeq-1 Envs HIV-1 vaccine (GLA-SE adjuvanted), HIV p24gag prime-boost plasmid DNA vaccine, arenavirus vector-based vaccines (Vaxwave, TheraT), MVA-BN HIV-1 vaccine regimen, UBI HIV gp120, mRNA based prophylactic vaccines, and TBL-1203HI.

In some embodiments, the additional therapeutic agent is birth control (i.e., a contraceptive). Examples of birth control include cyproterone acetate, desogestrel, dienogest, drospirenone, estradiol valerate, ethinyl Estradiol, ethynodiol, etonogestrel, levomefolate, levonorgestrel, lynestrenol, medroxyprogesterone acetate, mestranol, mifepristone, misoprostol, nomegestrol acetate, norelgestromin, norethindrone, noretynodrel, norgestimate, ormeloxifene, segestersone acetate, ulipristal acetate, and any combinations thereof.

In some embodiments, a provided composition is combined with one, two, three, four or more additional therapeutic agents selected from ATRIPLA® (efavirenz, tenofovir disoproxil fumarate, and emtricitabine); COMPLERA® (EVIPLERA®; rilpivirine, tenofovir disoproxil fumarate, and emtricitabine); STRIBILD® (elvitegravir, cobicistat, tenofovir disoproxil fumarate, and emtricitabine); TRUVADA® (tenofovir disoproxil fumarate and emtricitabine; TDF+FTC); DESCOVY® (tenofovir alafenamide and emtricitabine); ODEFSEY® (tenofovir alafenamide, emtricitabine, and rilpivirine); GENVOYA® (tenofovir alafenamide, emtricitabine, cobicistat, and elvitegravir); BIKTARVY (bictegravir+emtricitabine+tenofovir alafenamide), adefovir; adefovir dipivoxil; cobicistat; emtricitabine; tenofovir; tenofovir disoproxil; tenofovir disoproxil fumarate; tenofovir alafenamide; tenofovir alafenamide hemifumarate; TRIUMEQ® (dolutegravir, abacavir, and lamivudine); dolutegravir, abacavir sulfate, and lamivudine; raltegravir; raltegravir and lamivudine; maraviroc; enfuvirtide; ALUVIA® (KALETRA®; lopinavir and ritonavir); COMBIVIR® (zidovudine and lamivudine; AZT+3TC); EPZICOM® (LIVEXA®; abacavir sulfate and lamivudine; ABC+3TC); TRIZIVIR® (abacavir sulfate, zidovudine, and lamivudine; ABC+AZT+3TC); rilpivirine; rilpivirine hydrochloride; atazanavir sulfate and cobicistat; atazanavir and cobicistat; darunavir and cobicistat; atazanavir; atazanavir sulfate; dolutegravir; elvitegravir; ritonavir; atazanavir sulfate and ritonavir; darunavir; lamivudine; prolastin; fosamprenavir; fosamprenavir calcium efavirenz; etravirine; nelfinavir; nelfinavir mesylate; interferon; didanosine; stavudine; indinavir; indinavir sulfate; tenofovir and lamivudine; zidovudine; nevirapine; saquinavir; saquinavir mesylate; aldesleukin; zalcitabine; tipranavir; amprenavir; delavirdine; delavirdine mesylate; Radha-108 (receptol); lamivudine and tenofovir disoproxil fumarate; efavirenz, lamivudine, and tenofovir disoproxil fumarate; phosphazid; lamivudine, nevirapine, and zidovudine; abacavir; and abacavir sulfate.

In some embodiments, a provided composition is combined with an HIV nucleoside or nucleotide inhibitor of reverse transcriptase and an HIV non-nucleoside inhibitor of reverse transcriptase. In another specific embodiment, an agent disclosed herein, or a pharmaceutical composition thereof, is combined with an HIV nucleoside or nucleotide inhibitor of reverse transcriptase, and an HIV protease inhibiting compound. In an additional embodiment, an agent disclosed herein, or a pharmaceutical composition thereof, is combined with an HIV nucleoside or nucleotide inhibitor of reverse transcriptase, an HIV non-nucleoside inhibitor of reverse transcriptase, and a pharmacokinetic enhancer. In certain embodiments, an agent disclosed herein, or a pharmaceutical composition thereof, is combined with at least one HIV nucleoside inhibitor of reverse transcriptase, an integrase inhibitor, and a pharmacokinetic enhancer. In another embodiment, an agent disclosed herein, or a pharmaceutical composition thereof, is combined with two HIV nucleoside or nucleotide inhibitors of reverse transcriptase.

In some embodiments, a provided composition is combined with abacavir sulfate, tenofovir, tenofovir disoproxil, tenofovir disoproxil fumarate, tenofovir disoproxil hemifumarate, tenofovir alafenamide, or tenofovir alafenamide hemifumarate.

In some embodiments, a provided composition is combined with tenofovir, tenofovir disoproxil, tenofovir disoproxil fumarate, tenofovir alafenamide, or tenofovir alafenamide hemifumarate.

In some embodiments, a provided composition is combined with a first additional therapeutic agent selected from the group consisting of abacavir sulfate, tenofovir, tenofovir disoproxil, tenofovir disoproxil fumarate, tenofovir alafenamide, and tenofovir alafenamide hemifumarate, and a second additional therapeutic agent selected from the group consisting of emtricitabine and lamivudine.

In some embodiments, a provided composition is combined with a first additional therapeutic agent selected from the group consisting of tenofovir, tenofovir disoproxil, tenofovir disoproxil fumarate, tenofovir alafenamide, and tenofovir alafenamide hemifumarate, and a second additional therapeutic agent, wherein the second additional therapeutic agent is emtricitabine.

In some embodiments, a provided composition is combined with a first additional therapeutic agent (a contraceptive) selected from the group consisting of cyproterone acetate, desogestrel, dienogest, drospirenone, estradiol valerate, ethinyl Estradiol, ethynodiol, etonogestrel, levomefolate, levonorgestrel, lynestrenol, medroxyprogesterone acetate, mestranol, mifepristone, misoprostol, nomegestrol acetate, norelgestromin, norethindrone, noretynodrel, norgestimate, ormeloxifene, segestersone acetate, ulipristal acetate, and any combinations thereof.

In some embodiments, the additional therapeutic agent is gene therapy or cell therapy. Gene therapy and cell therapy include genetic modification to silence a gene; genetic approaches to directly kill the infected cells; the infusion of immune cells designed to replace most of the patient's own immune system to enhance the immune response to infected cells, or activate the patient's own immune system to kill infected cells, or find and kill the infected cells; genetic approaches to modify cellular activity to further alter endogenous immune responsiveness against the infection. Examples of dendritic cell therapy include AGS-004. CCR5 gene editing agents include SB-728T. CCR5 gene inhibitors include Cal-1. In some embodiments, C34-CCR5/C34-CXCR4 expressing CD4-positive T-cells are co-administered with one or more multi-specific antigen binding molecules. In some embodiments, the agents described herein are co-administered with AGT-103-transduced autologous T-cell therapy or AAV-eCD4-Ig gene therapy.

In some embodiments, the additional therapeutic agent is a gene editor (e.g., an HIV targeted gene editor). In some embodiments a genome editing system is selected from the group consisting of a CRISPR/Cas9 complex, a zinc finger nuclease complex, a TALEN complex, a homing endonucleases complex, and a meganuclease complex. An illustrative HIV targeting CRISPR/Cas9 system includes without limitation EBT-101.

In some embodiments, the additional therapeutic agent is CAR-T cell therapy. CAR-T cell therapy comprises a population of immune effector cells engineered to express a chimeric antigen receptor (CAR), wherein the CAR comprises an HIV antigen-binding domain. The HIV antigen includes an HIV envelope protein or a portion thereof, gp120 or a portion thereof, a CD4 binding site on gp120, the CD4-induced binding site on gp120, N glycan on gp120, the V2 of gp120, the membrane proximal region on gp41. The immune effector cell is a T-cell or an NK cell. In some embodiments, the T-cell is a CD4+ T-cell, a CD8+ T-cell, or a combination thereof. Cells can be autologous or allogeneic. Examples of HIV CAR-T include VC-CAR-T, CMV-N6-CART, anti-CD4 CART-cell therapy, CD4 CAR+C34-CXCR4+CCR5 ZFN T-cells, autologous hematopoietic stem cells genetically engineered to express a CD4 CAR and the C46 peptide.

In some embodiments, the additional therapeutic agent is TCR-T cell therapy. TCR-T cell therapy comprises TCR-T cells engineered to target HIV derived proteins present on the surface of virus-infected cells, for example ImmTAV.

In some embodiments, the antibodies or antigen-binding fragments described herein are combined with a population of B cells genetically modified to express broadly neutralizing antibodies, such as 3BNC117 (Hartweger et al, J. Exp. Med. 2019, 1301, Moffett et al., Sci. Immunol. 4, eaax0644 (2019) 17 May 2019).

### HBV Infection

The present disclosure provides methods of treating and/or preventing hepatitis B virus (HBV) infection in a subject in need thereof. In some embodiments, a method of treating and/or preventing HBV infection in a subject in need thereof comprises administering to the subject a composition provided herein.

In some embodiments, a method of treating HBV infection in a subject in need thereof comprises administering to the subject a composition provided herein.

In some embodiments, a method of preventing HBV infection in a subject in need thereof comprises administering to the subject a composition provided herein. In some such embodiments, the subject is at risk of acquiring HBV infection.

In some embodiments, the present disclosure provides compositions for use in the treatment and/or prevention of HBV infection in a subject.

In some embodiments, the present disclosure provides compositions for the manufacture of a medicament for treating and/or preventing HBV infection in a subject.

In some embodiments, the present disclosure provides a method of treating and/or preventing HBV infection in a subject in need thereof, comprising administering to the subject a combination therapy comprising a composition provided herein and one or more additional therapeutic agents. In some embodiments, the subject is receiving or has received one or more additional therapeutic agents. In some embodiments, the additional therapeutic agents are selected from the same class of therapeutic agents. In some embodiments, the additional therapeutic agents are selected from a different class of therapeutic agents. In some embodiments, the additional therapeutic agent is for treating and/or preventing HBV infection. In some embodiments, the additional therapeutic agent is not for treating and/or preventing HBV infection.

In some embodiments, the present disclosure provides a method for treating an HBV infection, comprising administering to a subject in need thereof a therapeutically effective amount of a composition disclosed herein, in combination with a therapeutically effective amount of one or more (e.g., one, two, three, four, one or two, one to three, or one to four) additional therapeutic agents which are suitable for treating an HBV infection.

In some embodiments, a composition disclosed herein is combined with one, two, three, four, or more additional therapeutic agents. In some embodiments, a composition disclosed herein is combined with two additional therapeutic agents. In some embodiments, a composition disclosed herein is combined with three additional therapeutic agents. In some embodiments, a composition disclosed herein is combined with four additional therapeutic agents. The one, two, three, four, or more additional therapeutic agents can be different therapeutic agents selected from the same class of therapeutic agents, and/or they can be selected from different classes of therapeutic agents.

The compositions described herein may be used or combined with one or more of a chemotherapeutic agent, an immunomodulator, an immunotherapeutic agent, a therapeutic antibody, a therapeutic vaccine, a bispecific antibody and “antibody-like” therapeutic protein (such as DARTs®, Duobodies®, Bites®, XmAbs®, TandAbs®, Fab derivatives), an antibody-drug conjugate (ADC), gene modifiers or gene editors (such as CRISPR Cas9, zinc finger nucleases, homing endonucleases, synthetic nucleases, TALENs), cell therapies such as CAR-T (chimeric antigen receptor T-cell), and TCR-T (an engineered T cell receptor) agent or any combination thereof.

In the some embodiments, the additional therapeutic agent may be an anti-HBV agent. For example, the additional therapeutic agent may be selected from the group consisting of HBV combination drugs, other drugs for treating HBV, 3-dioxygenase (IDO) inhibitors, antisense oligonucleotide targeting viral mRNA, Apolipoprotein A1 modulator, arginase inhibitors, B- and T-lymphocyte attenuator inhibitors, Bruton's tyrosine kinase (BTK) inhibitors, CCR2 chemokine antagonist, CD137 inhibitors, CD160 inhibitors, CD305 inhibitors, CD4 agonist and modulator, compounds targeting HBcAg, compounds targeting hepatitis B core antigen (HBcAg), covalently closed circular DNA (cccDNA) inhibitors, cyclophilin inhibitors, cytokines, cytotoxic T-lymphocyte-associated protein 4 (ipi4) inhibitors, DNA polymerase inhibitor, Endonuclease modulator, epigenetic modifiers, Farnesoid X receptor agonist, gene modifiers or editors, HBsAg inhibitors, HBsAg secretion or assembly inhibitors, HBV antibodies, HBV DNA polymerase inhibitors, HBV replication inhibitors, HBV RNAse inhibitors, HBV vaccines, HBV viral entry inhibitors, HBx inhibitors, Hepatitis B large envelope protein modulator, Hepatitis B large envelope protein stimulator, Hepatitis B structural protein modulator, hepatitis B surface antigen (HBsAg) inhibitors, hepatitis B surface antigen (HBsAg) secretion or assembly inhibitors, hepatitis B virus E antigen inhibitors, hepatitis B virus replication inhibitors, Hepatitis virus structural protein inhibitor, HIV-1 reverse transcriptase inhibitor, Hyaluronidase inhibitor, IAPB inhibitors, IL-2 agonist, IL-7 agonist, Immunoglobulin agonist, Immunoglobulin G modulator, immunomodulators, indoleamine-2, inhibitors of ribonucleotide reductase, Interferon agonist, Interferon alpha 1 ligand, Interferon alpha 2 ligand, Interferon alpha 5 ligand modulator, Interferon alpha ligand, Interferon alpha ligand modulator, interferon alpha receptor ligands, Interferon beta ligand, Interferon ligand, Interferon receptor modulator, Interleukin-2 ligand, ipi4 inhibitors, lysine demethylase inhibitors, histone demethylase inhibitors, KDM5 inhibitors, KDM1 inhibitors, killer cell lectin-like receptor subfamily G member 1 inhibitors, lymphocyte-activation gene 3 inhibitors, lymphotoxin beta receptor activators, microRNA (miRNA) gene therapy agents, modulators of Ax1, modulators of B7-H3, modulators of B7-H4, modulators of CD160, modulators of CD161, modulators of CD27, modulators of CD47, modulators of CD70, modulators of GITR, modulators of HEVEM, modulators of ICOS, modulators of Mer, modulators of NKG2A, modulators of NKG2D, modulators of OX40, modulators of SIRPalpha, modulators of TIGIT, modulators of Tim-4, modulators of Tyro, Na+-taurocholate cotransporting polypeptide (NTCP) inhibitors, natural killer cell receptor 2B4 inhibitors, NOD2 gene stimulator, Nucleoprotein inhibitor, nucleoprotein modulators, PD-1 inhibitors, PD-L1 inhibitors, PEG-Interferon Lambda, Peptidylprolyl isomerase inhibitor, phosphatidylinositol-3 kinase (PI3K) inhibitors, recombinant scavenger receptor A (SRA) proteins, recombinant thymosin alpha-1, Retinoic acid-inducible gene 1 stimulator, Reverse transcriptase inhibitor, Ribonuclease inhibitor, RNA DNA polymerase inhibitor, short interfering RNAs (siRNA), short synthetic hairpin RNAs (sshRNAs), SLC10A1 gene inhibitor, SMAC mimetics, Src tyrosine kinase inhibitor, stimulator of interferon gene (STING) agonists, stimulators of NOD1, T cell surface glycoprotein CD28 inhibitor, T-cell surface glycoprotein CD8 modulator, Thymosin agonist, Thymosin alpha 1 ligand, Tim-3 inhibitors, TLR-3 agonist, TLR-7 agonist, TLR-9 agonist, TLR9 gene stimulator, toll-like receptor (TLR) modulators, Viral ribonucleotide reductase inhibitor, zinc finger nucleases or synthetic nucleases (TALENs), and combinations thereof.

In some embodiments, provided compositions are combined with one, two, three, four or more additional therapeutic agents selected from HBV combination drugs, HBV vaccines, HBV DNA polymerase inhibitors, immunomodulators toll-like receptor (TLR) modulators, interferon alpha receptor ligands, hyaluronidase inhibitors, hepatitis b surface antigen (HBsAg) inhibitors, cytotoxic T-lymphocyte-associated protein 4 (ipi4) inhibitors, cyclophilin inhibitors, HBV viral entry inhibitors, antisense oligonucleotide targeting viral mRNA, short interfering RNAs (siRNA) and ddRNAi endonuclease modulators, ribonucelotide reductase inhibitors, HBV E antigen inhibitors, covalently closed circular DNA (cccDNA) inhibitors, farnesoid X receptor agonists, HBV antibodies, CCR2 chemokine antagonists, thymosin agonists, cytokines, nucleoprotein modulators, retinoic acid-inducible gene 1 stimulators, NOD2 stimulators, phosphatidylinositol 3-kinase (PI3K) inhibitors, indoleamine-2, 3-dioxygenase (IDO) pathway inhibitors, PD-1 inhibitors, PD-L1 inhibitors, recombinant thymosin alpha-1, bruton's tyrosine kinase (BTK) inhibitors, KDM inhibitors, HBV replication inhibitors, arginase inhibitors, and other HBV drugs.

In some embodiments, the additional therapeutic agent is a HBV combination drug. Examples of HBV combination drugs include TRUVADA® (tenofovir disoproxil fumarate and emtricitabine); ABX-203, lamivudine, and PEG-IFN-alpha; ABX-203 adefovir, and PEG-IFNalpha; and INO-1800 (INO-9112 and RG7944).

In some embodiments, the additional therapeutic agent is an other HBV drug. Examples of other drugs for the treatment of HBV infection include alpha-hydroxytropolones, amdoxovir, beta-hydroxycytosine nucleosides, AL-034, CCC-0975, elvucitabine, ezetimibe, cyclosporin A, gentiopicrin (gentiopicroside), JNJ-56136379, nitazoxanide, birinapant, NJK14047, NOV-205 (molixan, BAM-205), oligotide, mivotilate, feron, GST-HG-131, levamisole, Ka Shu Ning, alloferon, WS-007, Y-101 (Ti Fen Tai), rSIFN-co, PEG-IIFNm, KW-3, BP-Inter-014, oleanolic acid, HepB-nRNA, cTP-5 (rTP-5), HSK-II-2, HEISCO-106-1, HEISCO-106, Hepbarna, IBPB-006IA, Hepuyinfen, DasKloster 0014-01, ISA-204, Jiangantai (Ganxikang), MIV-210, OB-AI-004, PF-06, picroside, DasKloster-0039, hepulantai, IMB-2613, TCM-800B, reduced glutathione, RO-6864018, RG-7834, UB-551, and ZH-2N, and the compounds disclosed in US20150210682, (Roche), US 2016/0122344 (Roche), WO2015173164, WO2016023877, US2015252057A (Roche), WO16128335A1 (Roche), WO16120186A1 (Roche), US2016237090A (Roche), WO16107833A1 (Roche), WO16107832A1 (Roche), US2016176899A (Roche), WO16102438A1 (Roche), WO16012470A1 (Roche), US2016220586A (Roche), and US2015031687A (Roche).

In some embodiments, the additional therapeutic agent is a HBV vaccine. In some embodiments, the HBV vaccine is a prophylactic HBV vaccine. Examples of prophylactic HBV vaccines include Vaxelis, Hexaxim, Heplisav, Mosquirix, DTwP-HBV vaccine, Bio-Hep-B, D/T/P/HBV/M (LBVP-0101; LBVW-0101), DTwP-Hepb-Hib-IPV vaccine, Heberpenta L, DTwP-HepB-Hib, V-419, CVI-HBV-001, Tetrabhay, hepatitis B prophylactic vaccine (Advax Super D), Hepatrol-07, GSK-223192A, ENGERIX B®, recombinant hepatitis B vaccine (intramuscular, Kangtai Biological Products), recombinant hepatitis B vaccine (Hansenual polymorpha yeast, intramuscular, Hualan Biological Engineering), recombinant hepatitis B surface antigen vaccine, Bimmugen, Euforavac, Eutravac, anrix-DTaP-IPV-Hep B, HBAI-20, Infanrix-DTaP-IPV-Hep B-Hib, Pentabio Vaksin DTP-HB-Hib, Comvac 4, Twinrix, Euvax-B, Tritanrix HB, Infanrix Hep B, Comvax, DTP-Hib-HBV vaccine, DTP-HBV vaccine, Yi Tai, Heberbiovac HB, Trivac HB, GerVax, DTwP-Hep B-Hib vaccine, Bilive, Hepavax-Gene, SUPERVAX, Comvac5, Shanvac-B, Hebsulin, Recombivax HB, Revac B mcf, Revac B+, Fendrix, DTwP-HepB-Hib, DNA-001, Shan5, Shan6, rhHBsAG vaccine, HBI pentavalent vaccine, LBVD, Infanrix HeXa, and DTaP-rHB-Hib vaccine.

In some embodiments, the HBV vaccine is a therapeutic HBV vaccine. Examples of therapeutic HBV vaccines include HBsAG-HBIG complex, ARB-1598, Bio-Hep-B, NASVAC, abi-HB (intravenous), ABX-203, Tetrabhay, GX-110E, GS-4774, peptide vaccine (epsilonPA-44), Hepatrol-07, NASVAC (NASTERAP), IMP-321, BEVAC, Revac B mcf, Revac B+, MGN-1333, KW-2, CVI-HBV-002, AltraHepB, VGX-6200, FP-02, FP-02.2, TG-1050, NU-500, HBVax, im/TriGrid/antigen vaccine, Mega-CD40L-adjuvanted vaccine, HepB-v, RG7944 (INO-1800), recombinant VLP-based therapeutic vaccine (HBV infection, VLP Biotech), AdTG-17909, AdTG-17910 AdTG-18202, ChronVac-B, TG-1050, and Lm HBV.

In some embodiments, the additional therapeutic agent is a HBV DNA polymerase inhibitor. Examples of HBV DNA polymerase inhibitors include adefovir (HEPSERA®), emtricitabine (EMTRIVA®), tenofovir disoproxil fumarate (VIREAD®), tenofovir alafenamide, tenofovir, tenofovir disoproxil, tenofovir alafenamide fumarate, tenofovir alafenamide hemifumarate, tenofovir dipivoxil, tenofovir dipivoxil fumarate, tenofovir octadecyloxyethyl ester, CMX-157, besifovir, entecavir)(BARACLUDE®), entecavir maleate, telbivudine (TYZEKA®), pradefovir, clevudine, ribavirin, lamivudine (EPIVIR-HBV®), phosphazide, famciclovir, fusolin, metacavir, SNC-019754, FMCA, AGX-1009, AR-II-04-26, HIP-1302, tenofovir disoproxil aspartate, tenofovir disoproxil orotate, and HS-10234.

In some embodiments, the additional therapeutic agent is an immunomodulatory. Examples of immunomodulators include rintatolimod, imidol hydrochloride, ingaron, dermaVir, plaquenil (hydroxychloroquine), proleukin, hydroxyurea, mycophenolate mofetil (MPA) and its ester derivative mycophenolate mofetil (MMF), WF-10, ribavirin, IL-12, INO-9112, polymer polyethyleneimine (PEI), Gepon, VGV-1, MOR-22, BMS-936559, RO-7011785, RO-6871765, AIC-649, and IR-103.

In some embodiments, the additional therapeutic agent is a toll-like receptor (TLR) modulator. TLR modulators include modulators of TLR1, TLR2, TLR3, TLR4, TLR5, TLR6, TLR7, TLR8, TLR9, TLR10, TLR11, TLR12, and TLR13.

In some embodiments, the TLR modulator is a TLR3 modulator. Examples of TLR3 modulators include rintatolimod, poly-ICLC, RIBOXXON®, Apoxxim, RIBOXXIM®, IPH-33, MCT-465, MCT-475, GS-9688 and ND-1.1.

In some embodiments, the TLR modulator is a TLR7 modulator. Examples of TLR7 modulators include GS-9620, GSK-2245035, imiquimod, resiquimod, DSR-6434, DSP-3025, IMO-4200, MCT-465, MEDI-9197, 3M-051, SB-9922, 3M-052, Limtop, TMX-30X, TMX-202, RG-7863, RG-7795, RG-7854, and the compounds disclosed in US20100143301 (Gilead Sciences), US20110098248 (Gilead Sciences), and US20090047249 (Gilead Sciences).

In some embodiments, the TLR modulator is a TLR8 modulator. Examples of TLR8 modulators include motolimod, resiquimod, 3M-051, 3M-052, MCT-465, IMO-4200, VTX-763, VTX-1463, and the compounds disclosed in US20140045849 (Janssen), US20140073642 (Janssen), WO2014/056953 (Janssen), WO2014/076221 (Janssen), WO2014/128189 (Janssen), US20140350031 (Janssen), WO2014/023813 (Janssen), US20080234251 (Array Biopharma), US20080306050 (Array Biopharma), US20100029585 (Ventirx Pharma), US20110092485 (Ventirx Pharma), US20110118235 (Ventirx Pharma), US20120082658 (Ventirx Pharma), US20120219615 (Ventirx Pharma), US20140066432 (Ventirx Pharma), US20140088085 (Ventirx Pharma), US20140275167 (Novira Therapeutics), and US20130251673 (Novira Therapeutics).

In some embodiments, the TLR modulator is a TLR9 modulator. Examples of TLR9 modulators include BB-001, BB-006, CYT-003, IMO-2055, IMO-2125, IMO-3100, IMO-8400, IR-103, IMO-9200, agatolimod, DIMS-9054, DV-1079, DV-1179, AZD-1419, leftolimod (MGN-1703), litenimod, and CYT-003-QbG10.

In some embodiments, the additional therapeutic agent is an interferon alpha receptor ligand. Examples of interferon alpha receptor ligands include interferon alpha-2b (INTRON A®), pegylated interferon alpha-2a (PEGASYS®), PEGylated interferon alpha-1b, interferon alpha 1b (HAPGEN®), Veldona, Infradure, Roferon-A, YPEG-interferon alfa-2a (YPEG-rhlFNalpha-2a), P-1101, Algeron, Alfarona, Ingaron (interferon gamma), rSIFN-co (recombinant super compound interferon), Ypeginterferon alfa-2b (YPEG-rhlFNalpha-2b), MOR-22, peginterferon alfa-2b (PEG-INTRON®), Bioferon, Novaferon, Inmutag (Inferon), MULTIFERON®, interferon alfa-n1 (HUMOFERON®), interferon beta-1a)(AVONEX®), Shaferon, interferon alfa-2b (Axxo), Alfaferone, interferon alfa-2b (BioGeneric Pharma), interferon-alpha 2 (CJ), Laferonum, VIPEG, BLAUFERON-A, BLAUFERON-B, Intermax Alpha, Realdiron, Lanstion, Pegaferon, PDferon-B PDferon-B, interferon alfa-2b (IFN, Laboratorios Bioprofarma), alfainterferona 2b, Kalferon, Pegnano, Feronsure, PegiHep, interferon alfa 2b (Zydus-Cadila), interferon alfa 2a, Optipeg A, Realfa 2B, Reliferon, interferon alfa-2b (Amega), interferon alfa-2b (Virchow), ropeginterferon alfa-2b, rHSA-IFN alpha-2a (recombinant human serum albumin intereferon alpha 2a fusion protein), rHSA-IFN alpha 2b, recombinant human interferon alpha-(1b, 2a, 2b), peginterferon alfa-2b (Amega), peginterferon alfa-2a, Reaferon-EC, Proquiferon, Uniferon, Urifron, interferon alfa-2b (Changchun Institute of Biological Products), Anterferon, Shanferon, Layfferon, Shang Sheng Lei Tai, INTEFEN, SINOGEN, Fukangtai, Pegstat, rHSA-IFN alpha-2b, SFR-9216, and Interapo (Interapa).

In some embodiments, the additional therapeutic agent is a hyaluronidase inhibitor. An example of a hyaluronidase inhibitor is astodrimer.

In some embodiments, the additional therapeutic agent is a hepatitis B surface antigen (HBsAg) inhibitor. Examples of HBsAg inhibitors include HBF-0259, PBHBV-001, PBHBV-2-15, PBHBV-2-1, REP-9AC, REP-9C, REP-9, REP-2139, REP-2139-Ca, REP-2165, REP-2055, REP-2163, REP-2165, REP-2053, REP-2031 and REP-006, and REP-9AC′. An example of HBsAg secretion inhibitor is BM601.

In some embodiments, the additional therapeutic agent is a cytotoxic T-lymphocyte-associated protein 4 (ipi4) inhibitor. Examples of cytotoxic T-lymphocyte-associated protein 4 (ipi4) inhibitors include AGEN-2041, AGEN-1884, ipilumimab, belatacept, PSI-001, PRS-010, Probody mAbs, tremelimumab, and JHL-1155.

In some embodiments, the additional therapeutic agent is a cyclophilin inhibitor. Examples of cyclophilin inhibitors include CPI-431-32, EDP-494, OCB-030, SCY-635, NVP-015, NVP-018, NVP-019, STG-175, and the compounds disclosed in U.S. Pat. No. 8,513,184 (Gilead Sciences), US20140030221 (Gilead Sciences), US20130344030 (Gilead Sciences), and US20130344029 (Gilead Sciences).

In some embodiments, the additional therapeutic agent is a HBV viral entry inhibitor. An example of a HBV viral entry inhibitor is Myrcludex B.

In some embodiments, the additional therapeutic agent is an antisense oligonucleotide targeting viral mRNA. Examples of antisense oligonucleotide targeting viral mRNA include ISIS-HBVRx, IONIS-HBVRx, IONIS-GSK6-LRx, GSK-3389404, RG-6004.

In some embodiments, the additional therapeutic agent is a short interfering RNA (siRNA). Examples of siRNA include TKM-HBV (TKM-HepB), ALN-HBV, SR-008, HepB-nRNA, and ARC-520, ARC-521, ARB-1740, ARB-1467.

In some embodiments, the additional therapeutic agent is a DNA-directed RNA interference (ddRNAi). An example of ddRNAi is BB-HB-331.

In some embodiments, the additional therapeutic agent is an endonuclease modulator. An example of an endonuclease modulator is PGN-514.

In some embodiments, the additional therapeutic agent is a ribonucleotide reductase inhibitor. An example of a ribonucleotide reductase inhibitor is Trimidox.

In some embodiments, the additional therapeutic agent is an HBV E antigen inhibitor. An example of a HBV E antigen inhibitor is wogonin.

In some embodiments, the additional therapeutic agent is a covalently closed circular DNA (cccDNA) inhibitor. Examples of cccDNA inhibitors include BSBI-25 and CHR-101.

In some embodiments, the additional therapeutic agent is a farnesoid x receptor agonist. An example of a farnesoid x receptor agonist is EYP-001.

In some embodiments, the additional therapeutic agent is an HBV antibody. Examples of HBV antibodies targeting the surface antigens of the hepatitis B virus include GC-1102, XTL-17, XTL-19, KN-003, IV Hepabulin SN, and fully human monoclonal antibody therapy (hepatitis B virus infection, Humabs BioMed). Examples of HBV antibodies, including monoclonal antibodies and polyclonal antibodies, include Zutectra, Shang Sheng Gan Di, Uman Big (Hepatitis B Hyperimmune), Omri-Hep-B, Nabi-HB, Hepatect CP, HepaGam B, igantibe, Niuliva, CT-P24, hepatitis B immunoglobulin (intravenous, pH4, HBV infection, Shanghai RAAS Blood Products), and Fovepta (BT-088). Fully human monoclonal antibodies such as HBC-34.

In some embodiments, the additional therapeutic agent is a CCR2 chemokine antagonist. An example of a CCR2 chemokine antagonist is propagermanium.

In some embodiments, the additional therapeutic agent is a thymosin agonist. An example of a thymosin agonist is Thymalfasin, recombinant thymosin alpha 1 (GeneScience).

In some embodiments, the additional therapeutic agent is a cytokine. Examples of cytokines include recombinant IL-7, CYT-107, interleukin-2 (IL-2, Immunex), recombinant human interleukin-2 (Shenzhen Neptunus), IL-15, IL-21, IL-24, and celmoleukin.

In some embodiments, the additional therapeutic agent is a nucleoprotein modulator. In some embodiments, the nucleoprotein modulator is a HBV core or capsid protein inhibitor. Examples of nucleoprotein modulators include AB-423, AT-130, GLS4, NVR-1221, NVR-3778, BAY 41-4109, morphothiadine mesilate, JNJ-379, RG-7907, ABI-H0731, ABI-H2158 and DVR-23. Examples of capsid inhibitors include the compounds disclosed in

US20140275167 (Novira Therapeutics), US20130251673 (Novira Therapeutics), US20140343032 (Roche), WO2014037480 (Roche), US20130267517 (Roche), WO2014131847 (Janssen), WO2014033176 (Janssen), WO2014033170 (Janssen), WO2014033167 (Janssen), WO2015/059212 (Janssen), WO2015118057 (Janssen), WO2015011281 (Janssen), WO2014184365 (Janssen), WO2014184350 (Janssen), WO2014161888 (Janssen), WO2013096744 (Novira), US20150225355 (Novira), US20140178337 (Novira), US20150315159 (Novira), US20150197533 (Novira), US20150274652 (Novira), US20150259324, (Novira), US20150132258 (Novira), U.S. Pat. No. 9,181,288 (Novira), WO2014184350 (Janssen), WO2013144129 (Roche).

In some embodiments, the additional therapeutic agent is a stimulator of retinoic acid-inducible gene 1. Examples of stimulators of retinoic acid-inducible gene 1 include SB-9200, SB-40, SB-44, ORI-7246, ORI-9350, ORI-7537, ORI-9020, ORI-9198, and ORI-7170, RGT-100.

In some embodiments, the additional therapeutic agent is a stimulator of NOD2. An example of a stimulator of NOD2 is SB-9200.

In some embodiments, the additional therapeutic agent is a phosphatidylinositol 3-kinase (PI3K) inhibitor. Examples of PI3K inhibitors include idelalisib, ACP-319, AZD-8186, AZD-8835, buparlisib, CDZ-173, CLR-457, pictilisib, neratinib, rigosertib, rigosertib sodium, EN-3342, TGR-1202, alpelisib, duvelisib, IPI-549, UCB-5857, taselisib, XL-765, gedatolisib, ME-401, VS-5584, copanlisib, CAI orotate, perifosine, RG-7666, GSK-2636771, DS-7423, panulisib, GSK-2269557, GSK-2126458, CUDC-907, PQR-309, INCB-40093, pilaralisib, BAY-1082439, puquitinib mesylate, SAR-245409, AMG-319, RP-6530, ZSTK-474, MLN-1117, SF-1126, RV-1729, sonolisib, LY-3023414, SAR-260301, TAK-117, HMPL-689, tenalisib, voxtalisib, and CLR-1401.

In some embodiments, the additional therapeutic agent is an indoleamine-2, 3-dioxygenase (IDO) pathway inhibitor. Examples of IDO inhibitors include epacadostat (INCB24360), resminostat (4SC-201), indoximod, F-001287, SN-35837, NLG-919, GDC-0919, GBV-1028, GBV-1012, NKTR-218, and the compounds disclosed in US20100015178 (Incyte), US2016137652 (Flexus Biosciences, Inc.), WO2014073738 (Flexus Biosciences, Inc.), and WO2015188085 (Flexus Biosciences, Inc.).

In some embodiments, the additional therapeutic agent is a PD-1 inhibitor. Examples of PD-1 inhibitors include nivolumab, pembrolizumab, pidilizumab, BGB-108, SHR-1210, PDR-001, PF-06801591, IBI-308, GB-226, STI-1110, and mDX-400.

In some embodiments, the additional therapeutic agent is a PD-L1 inhibitor. Examples of PD-L1 inhibitors include atezolizumab, avelumab, AMP-224, MEDI-0680, RG-7446, GX-P2, durvalumab, KY-1003, KD-033, MSB-0010718C, TSR-042, ALN-PDL, STI-A1014, CX-072, and BMS-936559.

In some embodiments, provided compositions are combined with compounds such as those disclosed in WO2018026971, US20180044329, US20180044305, US20180044304, US20180044303, US20180044350, US20180057455, US20180057486, US20180045142, WO20180044963, WO2018044783, WO2018009505, WO20180044329, WO2017066227, WO2017087777, US20170145025, WO2017079669, WO2017070089, US2017107216, WO2017222976, US20170262253, WO2017205464, US20170320875, WO2017192961, WO2017112730, US20170174679, WO2017106634, WO2017202744, WO2017202275, WO2017202273, WO2017202274, WO2017202276, WO2017180769, WO2017118762, WO2016041511, WO2016039749, WO2016142835, WO2016142852, WO2016142886, WO2016142894, and WO2016142833.

In some embodiments, the additional therapeutic agent is a recombinant thymosin alpha-1. Examples of recombinant thymosin alpha-1 include NL-004 and PEGylated thymosin alpha-1.

In some embodiments, the additional therapeutic agent is a Bruton's tyrosine kinase (BTK) inhibitor. Examples of BTK inhibitors include ABBV-105, acalabrutinib (ACP-196), ARQ-531, BMS-986142, dasatinib, ibrutinib, GDC-0853, PRN-1008, SNS-062, ONO-4059, BGB-3111, ML-319, MSC-2364447, RDX-022, X-022, AC-058, RG-7845, spebrutinib, TAS-5315, TP-0158, TP-4207, HM-71224, KBP-7536, M-2951, TAK-020, AC-0025, and the compounds disclosed in US20140330015 (Ono Pharmaceutical), US20130079327 (Ono Pharmaceutical), and US20130217880 (Ono Pharmaceutical).

In some embodiments, the additional therapeutic agent is a KDM inhibitor. In some embodiments, the KDM inhibitor is a KDM5 inhibitor. Examples of KDM5 inhibitors include the compounds disclosed in WO2016057924 (Genentech/Constellation Pharmaceuticals), US20140275092 (Genentech/Constellation Pharmaceuticals), US20140371195 (Epitherapeutics) and US20140371214 (Epitherapeutics), US20160102096 (Epitherapeutics), US20140194469 (Quanticel), US20140171432, US20140213591 (Quanticel), US20160039808 (Quanticel), US20140275084 (Quanticel), WO2014164708 (Quanticel). In some embodiments, the KDM inhibitor is a KDM1 inhibitor. Examples of KDM1 inhibitors include the compounds disclosed in U.S. Pat. No. 9,186,337B2 (Oryzon Genomics), and GSK-2879552, RG-6016, ORY-2001.

In some embodiments, the additional therapeutic agent is a hepatitis B virus replication inhibitor. Examples of hepatitis B virus replication inhibitors include isothiafludine, IQP-HBV, RM-5038, and Xingantie.

In some embodiments, the additional therapeutic agent is an arginase inhibitor. Examples of arginase inhibitors include CB-1158, C-201, and resminostat.

In some embodiments, combination therapy described herein comprises gene therapy and/or cell therapy. Gene therapy and cell therapy includes: genetic modification to silence a gene; genetic approaches to directly kill infected cells; infusion of immune cells designed to replace most of the subject's own immune system to enhance the immune response to infected cells, or activate the patient's own immune system to kill infected cells, or find and kill the infected cells; and genetic approaches to modify cellular activity to further alter endogenous immune responsiveness against the infection.

In some embodiments, combination therapy described herein comprises gene editors. The genome editing system can by selected from the group consisting of: a CRISPR/Cas9 system, a zinc finger nuclease system, a TALEN system, a homing endonucleases system, and a meganuclease system; e.g., cccDNA elimination via targeted cleavage, and altering one or more of the hepatitis B virus (HBV) viral genes. Altering (e.g., knocking out and/or knocking down) the PreC, C, X, PreSI, PreS2, S, P or SP gene refers to (1) reducing or eliminating PreC, C, X PreSI, PreS2, S, P or SP gene expression, (2) interfering with Precore, Core, X protein, Long surface protein, middle surface protein, S protein (also known as HBs antigen and HBsAg), polymerase protein, and/or Hepatitis B spliced protein function (HBe, HBc, HBx, PreS1, PreS2, S, Pol, and/or HBSP or (3) reducing or eliminating the intracellular, serum and/or intraparenchymal levels of HBe, HBc, HBx, LHBs, MHBs, SHBs, Pol, and/or HBSP proteins. Knockdown of one or more of the PreC, C, X, PreSI, PreS2, S, P and/or SP gene(s) is performed by targeting the gene(s) within HBV cccDNA and/or integrated HBV DNA.

In some embodiments, combination therapy described herein comprises CAR-T cell therapy. CAR-T cell therapy can comprise a population of immune effector cells engineered to express a chimeric antigen receptor (CAR), wherein the CAR comprises an HBV antigen-binding domain. The immune effector cell is a T cell or an NK cell. In some embodiments, the T cell is a CD4+ T cell, a CD8+ T cell, or a combination thereof. Cells can be autologous or allogeneic.

In some embodiments, combination therapy described herein comprises TCR-T cell therapy. TCR-T cell therapy can comprise: T cells expressing HBV-specific T cell receptors. TCR-T cells are engineered to target HBV derived peptides presented on the surface of virus-infected cells. T-Cells expressing HBV surface antigen (HBsAg)-specific TCR. TCR-T therapy directed to treatment of HBV, such as LTCR-H2-1.

In some embodiments, a method of treating and/or preventing HBV infection comprises administering combination therapy comprising a provided composition and one, two, three, or four additional therapeutic agents selected from the group consisting of adefovir (HEPSERA®), tenofovir disoproxil fumarate (VIREAD®), tenofovir alafenamide, tenofovir, tenofovir disoproxil, tenofovir alafenamide fumarate, tenofovir alafenamide hemifumarate, entecavir (BARACLUDE®), telbivudine (TYZEKA®), or lamivudine (EPIVIR-HBV®).

In some embodiments, a method of treating and/or preventing HBV infection comprises administering combination therapy comprising a provided composition and a first additional therapeutic agent selected from the group consisting of adefovir (HEPSERA®), tenofovir disoproxil fumarate (VIREAD®), tenofovir alafenamide, tenofovir, tenofovir disoproxil, tenofovir alafenamide fumarate, tenofovir alafenamide hemifumarate, entecavir (BARACLUDE®), telbivudine (TYZEKA®), or lamivudine (EPIVIR-HBV®).

In some embodiments, a method of treating and/or preventing HBV infection comprises administering combination therapy comprising a provided composition and an HBV DNA polymerase inhibitor. In some embodiments, a method of treating and/or preventing HBV infection comprises administering combination therapy comprising a provided composition, an HBV DNA polymerase inhibitor and at least one additional therapeutic agent selected from the group consisting of: immunomodulators, TLR modulators, interferon alpha receptor ligands, hyaluronidase inhibitors, recombinant IL-7, HBsAg inhibitors, HBsAg secretion or assembly inhibitors, compounds targeting HBcAg, cyclophilin inhibitors, HBV vaccines, HBV viral entry inhibitors, NTCP inhibitors, antisense oligonucleotide targeting viral mRNA, siRNA, miRNA gene therapy agents, endonuclease modulators, inhibitors of ribonucleotide reductase, hepatitis B virus E antigen inhibitors, recombinant SRA proteins, src kinase inhibitors, HBx inhibitors, cccDNA inhibitors, sshRNAs, HBV antibodies including HBV antibodies targeting the surface antigens of the hepatitis B virus and bispecific antibodies and “antibody-like” therapeutic proteins (such as DARTs®, DUOBODIES®, BITES®, XmAbs®, TandAbs®, Fab derivatives, or TCR-like antibodies), CCR2 chemokine antagonists, thymosin agonists, cytokines, nucleoprotein modulators (HBV core or capsid protein modulators), stimulators of retinoic acid-inducible gene 1, stimulators of RIG-I like receptors, stimulators of NOD2, stimulators of NOD1, Arginase inhibitors, STING agonists, PI3K inhibitors, lymphotoxin beta receptor activators, natural killer cell receptor 2B4 inhibitors, Lymphocyte-activation gene 3 inhibitors, CD160 inhibitors, cytotoxic T-lymphocyte-associated protein 4 (ipi4) inhibitors, CD137 inhibitors, Killer cell lectin-like receptor subfamily G member 1 inhibitors, TIM-3 inhibitors, B- and T-lymphocyte attenuator inhibitors, CD305 inhibitors, PD-1 inhibitors, PD-L1 inhibitors, PEG-Interferon Lambda, recombinant thymosin alpha-1, BTK inhibitors, modulators of TIGIT, modulators of CD47, modulators of SIRPalpha, modulators of ICOS, modulators of CD27, modulators of CD70, modulators of OX40, epigenetic modifiers, modulators of NKG2D, modulators of Tim-4, modulators of B7-H4, modulators of B7-H3, modulators of NKG2A, modulators of GITR, modulators of CD160, modulators of HEVEM, modulators of CD161, modulators of Axl, modulators of Mer, modulators of Tyro, gene modifiers or editors such as CRISPR (including CRISPR Cas9), zinc finger nucleases or synthetic nucleases (TALENs), IAPB inhibitors, SMAC mimetics, KDM5 inhibitors, IDO inhibitors, and hepatitis B virus replication inhibitors.

In some embodiments, a method of treating and/or preventing HBV infection comprises administering combination therapy comprising (i) a provided composition, (ii) an HBV DNA polymerase inhibitor, (iii) one or two additional therapeutic agents selected from the group consisting of immunomodulators, TLR modulators, HBsAg inhibitors, HBsAg secretion or assembly inhibitors, HBV therapeutic vaccines, HBV antibodies including HBV antibodies targeting the surface antigens of the hepatitis B virus and bispecific antibodies and “antibody-like” therapeutic proteins (such as DARTs®, DUOBODIES®, BITES®, XmAbs®, TandAbs®, Fab derivatives, or TCR-like antibodies), cyclophilin inhibitors, stimulators of retinoic acid-inducible gene 1, stimulators of RIG-I like receptors, PD-1 inhibitors, PD-L1 inhibitors, Arginase inhibitors, PI3K inhibitors, IDO inhibitors, and stimulators of NOD2, and one or two additional therapeutic agents selected from the group consisting of HBV viral entry inhibitors, NTCP inhibitors, HBx inhibitors, cccDNA inhibitors, HBV antibodies targeting the surface antigens of the hepatitis B virus, siRNA, miRNA gene therapy agents, sshRNAs, KDM5 inhibitors, and nucleoprotein modulators (HBV core or capsid protein modulators).

In some embodiments, a method of treating and/or preventing HBV infection comprises administering combination therapy comprising a provided composition, an HBV DNA polymerase inhibitor, and at least a second additional therapeutic agent selected from the group consisting of immunomodulators, TLR modulators, HBsAg inhibitors, HBV therapeutic vaccines, HBV antibodies including HBV antibodies targeting the surface antigens of the hepatitis B virus and bispecific antibodies and “antibody-like” therapeutic proteins (such as DARTs® DUOBODIES®, BITES®, XmAbs®, TandAbs®, Fab derivatives, or TCR-like antibodies), cyclophilin inhibitors, stimulators of retinoic acid-inducible gene 1, stimulators of RIG-I like receptors, PD-1 inhibitors, PD-L1 inhibitors, Arginase inhibitors, PI3K inhibitors, IDO inhibitors, and stimulators of NOD2.

In some embodiments, a method of treating and/or preventing HBV infection comprises administering combination therapy comprising a provided composition, an HBV DNA polymerase inhibitor, and at least a second additional therapeutic agent selected from the group consisting of: HBV viral entry inhibitors, NTCP inhibitors, HBx inhibitors, cccDNA inhibitors, HBV antibodies targeting the surface antigens of the hepatitis B virus, siRNA, miRNA gene therapy agents, sshRNAs, KDM5 inhibitors, and nucleoprotein modulators (HBV core or capsid protein inhibitors).

In some embodiments, a method of treating and/or preventing HBV infection comprises administering combination therapy comprising (i) a provided composition; (ii) a first additional therapeutic agent selected from the group consisting of adefovir (HEPSERA®), tenofovir disoproxil fumarate (VIREAD®), tenofovir alafenamide, tenofovir, tenofovir disoproxil, tenofovir alafenamide fumarate, tenofovir alafenamide hemifumarate, entecavir (BARACLUDE®), telbivudine (TYZEKA®), or lamivudine (EPIVIR-HBV®), and at least a second additional therapeutic agent selected from the group consisting of immunomodulators, TLR modulators, interferon alpha receptor ligands, hyaluronidase inhibitors, recombinant IL-7, HBsAg inhibitors, HBsAg secretion or assembly inhibitors, compounds targeting HBcAg, cyclophilin inhibitors, HBV vaccines, HBV viral entry inhibitors, NTCP inhibitors, antisense oligonucleotide targeting viral mRNA, siRNA, miRNA gene therapy agents, endonuclease modulators, inhibitors of ribonucleotide reductase, hepatitis B virus E antigen inhibitors, recombinant SRA proteins, src kinase inhibitors, HBx inhibitors, cccDNA inhibitors, sshRNAs, HBV antibodies including HBV antibodies targeting the surface antigens of the hepatitis B virus and bispecific antibodies and “antibody-like” therapeutic proteins (such as DARTs®, DUOBODIES®, BITES®, XmAbs®, TandAbs®, Fab derivatives, and TCR-like antibodies), CCR2 chemokine antagonists, thymosin agonists, cytokines, nucleoprotein modulators (HBV core or capsid protein modulators), stimulators of retinoic acid-inducible gene 1, stimulators of RIG-I like receptors, stimulators of NOD2, stimulators of NOD1, IDO inhibitors, recombinant thymosin alpha-1, Arginase inhibitors, STING agonists, PI3K inhibitors, lymphotoxin beta receptor activators, natural killer cell receptor 2B4 inhibitors, Lymphocyte-activation gene 3 inhibitors, CD160 inhibitors, ipi4 inhibitors, CD137 inhibitors, killer cell lectin-like receptor subfamily G member 1 inhibitors, TIM-3 inhibitors, B- and T-lymphocyte attenuator inhibitors, epigenetic modifiers, CD305 inhibitors, PD-1 inhibitors, PD-L1 inhibitors, PEG-Interferon Lambd, BTK inhibitors, modulators of TIGIT, modulators of CD47, modulators of SIRPalpha, modulators of ICOS, modulators of CD27, modulators of CD70, modulators of OX40, modulators of NKG2D, modulators of Tim-4, modulators of B7-H4, modulators of B7-H3, modulators of NKG2A, modulators of GITR, modulators of CD160, modulators of HEVEM, modulators of CD161, modulators of Axl, modulators of Mer, modulators of Tyro, gene modifiers or editors such as CRISPR (including CRISPR Cas9), zinc finger nucleases or synthetic nucleases (TALENs), IAPB inhibitors, SMAC mimetics, KDM5 inhibitors, and hepatitis B virus replication inhibitors.

In some embodiments, a method of treating and/or preventing HBV infection comprises administering combination therapy comprising (i) a provided composition; (ii) a first additional therapeutic agent selected from the group consisting of adefovir (HEPSERA®), tenofovir disoproxil fumarate (VIREAD®), tenofovir alafenamide, tenofovir, tenofovir disoproxil, tenofovir alafenamide fumarate, tenofovir alafenamide hemifumarate, entecavir (BARACLUDE®), telbivudine (TYZEKA®) or lamivudine (EPIVIR-HBV®); and (iii) at least a second additional therapeutic agent selected from the group consisting of peginterferon alfa-2b (PEG-INTRON®), MULTIFERON®, interferon alpha 1b (HAPGEN®), interferon alpha-2b (INTRON A®), pegylated interferon alpha-2a)(PEGASYS®), interferon alfa-n1 (HUMOFERON®), ribavirin, interferon beta-1a (AVONEX®), Bioferon, Ingaron, Inmutag (Inferon), Algeron, Roferon-A, Oligotide, Zutectra, Shaferon, interferon alfa-2b (AXXO), Alfaferone, interferon alfa-2b (BioGeneric Pharma), Feron, interferon-alpha 2 (CJ), BEVAC, Laferonum, VIPEG, BLAUFERON-B, BLAUFERON-A, Intermax Alpha, Realdiron, Lanstion, Pegaferon, PDferon-B, interferon alfa-2b (IFN, Laboratorios Bioprofarma), alfainterferona 2b, Kalferon, Pegnano, Feronsure, PegiHep, interferon alfa 2b (Zydus-Cadila), Optipeg A, Realfa 2B, Reliferon, interferon alfa-2b (Amega), interferon alfa-2b (Virchow), peginterferon alfa-2b (Amega), Reaferon-EC, Proquiferon, Uniferon, Urifron, interferon alfa-2b (Changchun Institute of Biological Products), Anterferon, Shanferon, MOR-22, interleukin-2 (IL-2, Immunex), recombinant human interleukin-2 (Shenzhen Neptunus), Layfferon, Ka Shu Ning, Shang Sheng Lei Tai, INTEFEN, SINOGEN, Fukangtai, Alloferon, and celmoleukin.

In some embodiments, a method of treating and/or preventing HBV infection comprises administering combination therapy comprising (i) a provided composition; (ii) a first additional therapeutic agent selected from the group consisting of adefovir (HEPSERA®), tenofovir disoproxil fumarate (VIREAD®), tenofovir alafenamide, tenofovir, tenofovir disoproxil, tenofovir alafenamide fumarate, tenofovir alafenamide hemifumarate, entecavir (BARACLUDE®), telbivudine (TYZEKA®), or lamivudine (EPIVIR-HBV®); and (iii) at least a second additional therapeutic agent selected from the group consisting of immunomodulators, TLR modulators, HBsAg inhibitors, HBsAg secretion or assembly inhibitors, HBV therapeutic vaccines, HBV antibodies including HBV antibodies targeting the surface antigens of the hepatitis B virus and bispecific antibodies and “antibody-like” therapeutic proteins (such as DARTs®, DUOBODIES®, BITES®, XmAbs®, TandAbs®, Fab derivatives, or TCR-like antibodies), cyclophilin inhibitors, stimulators of retinoic acid-inducible gene 1, stimulators of RIG-I like receptors, Arginase inhibitors, PI3K inhibitors, PD-1 inhibitors, PD-L1 inhibitors, IDO inhibitors, and stimulators of NOD2.

In some embodiments, a method of treating and/or preventing HBV infection comprises administering combination therapy comprising (i) a provided composition; (ii) a first additional therapeutic agent selected from the group consisting of: adefovir (HEPSERA®), tenofovir disoproxil fumarate (VIREAD®), tenofovir alafenamide, tenofovir, tenofovir disoproxil, tenofovir alafenamide fumarate, tenofovir alafenamide hemifumarate, entecavir (BARACLUDE®), telbivudine (TYZEKA®), or lamivudine (EPIVIR-HBV®); and (iii) at least a second additional therapeutic agent selected from the group consisting of HBV viral entry inhibitors, NTCP inhibitors, HBx inhibitors, cccDNA inhibitors, HBV antibodies targeting the surface antigens of the hepatitis B virus, siRNA, miRNA gene therapy agents, sshRNAs, KDM5 inhibitors, and nucleoprotein modulators (HBV core or capsid protein modulators).

In some embodiments, a method of treating and/or preventing HBV infection comprises administering combination therapy comprising (i) a provided composition; (ii) a first additional therapeutic agent selected from the group consisting of adefovir (HEPSERA®), tenofovir disoproxil fumarate (VIREAD®), tenofovir alafenamide, tenofovir, tenofovir disoproxil, tenofovir alafenamide fumarate, tenofovir alafenamide hemifumarate, entecavir (BARACLUDE®), telbivudine (TYZEKA®), or lamivudine (EPIVIR-HBV®); (iii) one, two, or three additional therapeutic agents selected from the group consisting of immunomodulators, TLR modulators, HBsAg inhibitors, HBsAg secretion or assembly inhibitors, HBV therapeutic vaccines, HBV antibodies including HBV antibodies targeting the surface antigens of the hepatitis B virus and bispecific antibodies and “antibody-like” therapeutic proteins (such as DARTs®, DUOBODIES®, BITES®, XmAbs®, TandAbs®, Fab derivatives, or TCR-like antibodies), cyclophilin inhibitors, stimulators of retinoic acid-inducible gene 1, stimulators of RIG-I like receptors, PD-1 inhibitors, PD-L1 inhibitors, Arginase inhibitors, PI3K inhibitors, IDO inhibitors, and stimulators of NOD2; and (iv) one or two additional therapeutic agents selected from the group consisting of HBV viral entry inhibitors, NTCP inhibitors, HBx inhibitors, cccDNA inhibitors, HBV antibodies targeting the surface antigens of the hepatitis B virus, siRNA, miRNA gene therapy agents, sshRNAs, KDM5 inhibitors, and nucleoprotein modulators (HBV core or capsid protein modulators).

In some embodiments, a method of treating and/or preventing HBV infection comprises administering combination therapy comprising (i) a provided composition; (ii) a first additional therapeutic agent selected from the group consisting of adefovir (HEPSERA®), tenofovir disoproxil fumarate (VIREAD®), tenofovir alafenamide, tenofovir, tenofovir disoproxil, tenofovir alafenamide fumarate, tenofovir alafenamide hemifumarate, entecavir (BARACLUDE®), telbivudine (TYZEKA®), or lamivudine (EPIVIR-HBV®); (iii) one or two additional therapeutic agents selected from the group consisting of immunomodulators, TLR modulators, HBsAg inhibitors, HBsAg secretion or assembly inhibitors, HBV therapeutic vaccines, HBV antibodies including HBV antibodies targeting the surface antigens of the hepatitis B virus and bispecific antibodies and “antibody-like” therapeutic proteins (such as DARTs®, DUOBODIES®, BITES®, XmAbs®, TandAbs®, Fab derivatives, or TCR-like antibodies), cyclophilin inhibitors, stimulators of retinoic acid-inducible gene 1, stimulators of RIG-I like receptors, PD-1 inhibitors, PD-L1 inhibitors, Arginase inhibitors, PI3K inhibitors, IDO inhibitors, and stimulators of NOD2; and (iv) one or two additional therapeutic agents selected from the group consisting of HBV viral entry inhibitors, NTCP inhibitors, HBx inhibitors, cccDNA inhibitors, HBV antibodies targeting the surface antigens of the hepatitis B virus, siRNA, miRNA gene therapy agents, sshRNAs, KDM5 inhibitors, and nucleoprotein modulators (HBV core or capsid protein modulators).

In some embodiments, a method of treating and/or preventing HBV infection comprises administering combination therapy comprising (i) a provided composition; (ii) a first additional therapeutic agent selected from the group consisting of adefovir (HEPSERA®), tenofovir disoproxil fumarate (VIREAD®), tenofovir alafenamide, tenofovir, tenofovir disoproxil, tenofovir alafenamide fumarate, tenofovir alafenamide hemifumarate, entecavir (BARACLUDE®), telbivudine (TYZEKA®), or lamivudine (EPIVIR-HBV®); and (iii) one, two, three, or four additional therapeutic agents selected from the group consisting of immunomodulators, TLR7 modulators, TLR8 modulators, HBsAg inhibitors, HBsAg secretion or assembly inhibitors, HBV therapeutic vaccines, HBV antibodies including HBV antibodies targeting the surface antigens of the hepatitis B virus and bispecific antibodies and “antibody-like” therapeutic proteins (such as DARTs®, DUOBODIES®, BITES®, XmAbs®, TandAbs®, Fab derivatives, or TCR-like antibodies), cyclophilin inhibitors, stimulators of retinoic acid-inducible gene 1, stimulators of RIG-I like receptors, PD-1 inhibitors, PD-L1 inhibitors, Arginase inhibitors, PI3K inhibitors, IDO inhibitors, stimulators of NOD2 HBV viral entry inhibitors, NTCP inhibitors, HBx inhibitors, cccDNA inhibitors, siRNA, miRNA gene therapy agents, sshRNAs, KDM5 inhibitors, and nucleoprotein modulators (HBV core or capsid protein modulators).

In some embodiments, a method of treating and/or preventing HBV infection comprises administering combination therapy comprising a provided composition and one or more compounds such as those disclosed in U.S. Publication No. 2010/0143301 (Gilead Sciences), U.S. Publication No. 2011/0098248 (Gilead Sciences), U.S. Publication No. 2009/0047249 (Gilead Sciences), U.S. Pat. No. 8,722,054 (Gilead Sciences), U.S. Publication No. 2014/0045849 (Janssen), U.S. Publication No. 2014/0073642 (Janssen), WO2014/056953 (Janssen), WO2014/076221 (Janssen), WO2014/128189 (Janssen), U.S. Publication No. 2014/0350031 (Janssen), WO2014/023813 (Janssen), U.S. Publication No. 2008/0234251 (Array Biopharma), U.S. Publication No. 2008/0306050 (Array Biopharma), U.S. Publication No. 2010/0029585 (Ventirx Pharma), U.S. Publication No. 2011/0092485 (Ventirx Pharma), US2011/0118235 (Ventirx Pharma), U.S. Publication No. 2012/0082658 (Ventirx Pharma), U.S. Publication No. 2012/0219615 (Ventirx Pharma), U.S. Publication No. 2014/0066432 (Ventirx Pharma), U.S. Publication No. 2014/0088085 (Ventirx Pharma), U.S. Publication No. 2014/0275167 (Novira Therapeutics), U.S. Publication No. 2013/0251673 (Novira Therapeutics), U.S. Pat. No. 8,513,184 (Gilead Sciences), U.S. Publication No. 2014/0030221 (Gilead Sciences), U.S. Publication No. 2013/0344030 (Gilead Sciences), U.S. Publication No. 2013/0344029 (Gilead Sciences), US20140275167 (Novira Therapeutics), US20130251673 (Novira Therapeutics), U.S. Publication No. 2014/0343032 (Roche), WO2014037480 (Roche), U.S. Publication No. 2013/0267517 (Roche), WO2014131847 (Janssen), WO2014033176 (Janssen), WO2014033170 (Janssen), WO2014033167 (Janssen), WO2015/059212 (Janssen), WO2015118057 (Janssen), WO2015011281 (Janssen), WO2014184365 (Janssen), WO2014184350 (Janssen), WO2014161888 (Janssen), WO2013096744 (Novira), US20150225355 (Novira), US20140178337 (Novira), US20150315159 (Novira), US20150197533 (Novira), US20150274652 (Novira), US20150259324, (Novira), US20150132258 (Novira), U.S. Pat. No. 9,181,288 (Novira), WO2014184350 (Janssen), WO2013144129 (Roche), US20100015178 (Incyte), US2016137652 (Flexus Biosciences, Inc.), WO2014073738 (Flexus Biosciences, Inc.), WO2015188085 (Flexus Biosciences, Inc.), U.S. Publication No. 2014/0330015 (Ono Pharmaceutical), U.S. Publication No. 2013/0079327 (Ono Pharmaceutical), U.S. Publication No. 2013/0217880 (Ono pharmaceutical), WO2016057924 (Genentech/Constellation Pharmaceuticals), US20140275092 (Genentech/Constellation Pharmaceuticals), US20140371195 (Epitherapeutics) and US20140371214 (Epitherapeutics), US20160102096 (Epitherapeutics), US20140194469 (Quanticel), US20140171432, US20140213591 (Quanticel), US20160039808 (Quanticel), US20140275084 (Quanticel), WO2014164708 (Quanticel), U.S. Pat. No. 9,186,337B2 (Oryzon Genomics), and other drugs for treating HBV, and combinations thereof.

In certain embodiments, provided compositions may be combined with one or more (e.g., one, two, three, four, one or two, one to three, or one to four) additional therapeutic agents in any dosage amount of the provided composition.

In some embodiments, a provided composition is combined with 5-30 mg tenofovir alafenamide fumarate, tenofovir alafenamide hemifumarate, or tenofovir alafenamide. In some embodiments, a provided composition is combined with 5-10; 5-15; 5-20; 5-25; 25-30; 20-30; 15-30; or 10-30 mg tenofovir alafenamide fumarate, tenofovir alafenamide hemifumarate, or tenofovir alafenamide. In some embodiments, a provided composition is combined with 10 mg tenofovir alafenamide fumarate, tenofovir alafenamide hemifumarate, or tenofovir alafenamide. In some embodiments, a provided composition is combined with 25 mg tenofovir alafenamide fumarate, tenofovir alafenamide hemifumarate, or tenofovir alafenamide. A provided composition may be combined with agents provided herein in any dosage amount of the composition, the same as if each combination of dosages were specifically and individually listed.

In some embodiments, a provided composition is combined with 100-400 mg tenofovir disoproxil fumarate, tenofovir disoproxil hemifumarate, or tenofovir disoproxil. In some embodiments, a provided composition is combined with 100 mg to 150 mg; 100 mg to 200 mg; 100 mg to 250 mg; 100 mg to 300 mg; 100 mg to 350 mg; 150 mg to 200 mg; 150 mg to 250 mg; 150 mg to 300 mg; 150 mg to 350 mg; 150 mg to 400 mg; 200 mg to 250 mg; 200 mg to 300 mg; 200 mg to 350 mg; 200 mg to 400 mg; 250 mg to 350 mg; 250 mg to 400 mg; 350 mg to 400 or 300 mg to 400 mg tenofovir disoproxil fumarate, tenofovir disoproxil hemifumarate, or tenofovir disoproxil. In some embodiments, a provided composition is combined with 300 mg tenofovir disoproxil fumarate, tenofovir disoproxil hemifumarate, or tenofovir disoproxil. In some embodiments, a provided composition is combined with 250 mg tenofovir disoproxil fumarate, tenofovir disoproxil hemifumarate, or tenofovir disoproxil. In some embodiments, a provided composition is combined with 150 mg tenofovir disoproxil fumarate, tenofovir disoproxil hemifumarate, or tenofovir disoproxil. A provided composition may be combined with agents provided herein in any dosage amount of the provided composition, the same as if each combination of dosages were specifically and individually listed.

In some embodiments, kits comprising a composition disclosed herein in combination with one or more (e.g., one, two, three, four, one or two, or one to three, or one to four) additional therapeutic agents are provided. Provided compositions may be used in the kits, the same as if each and every composition were specifically and individually listed for use in a kit.

### Dosing and Administration

The present disclosure also provides compositions comprising a tenofovir agent in various forms for administration, which are useful in the methods described herein.

In some embodiments, provided compositions are formulated for subcutaneous, intramuscular, or parenteral administration. Accordingly, in some embodiments, provided methods comprise administering a provided composition subcutaneously, intramuscularly, or parenterally.

In some embodiments, administration of provided compositions is accomplished with a syringe and needle, pump, patch-pump, bolus injector, infusion, auto-injector, needle-free injector, or the like. Accordingly, the present disclosure also provides a receptacle containing a provided composition. In some embodiments, the receptacle is a syringe, pump, patch-pump, bolus injector, infusion, auto-injector, or needle-free injector.

In some embodiments, administration of provided compositions is accomplished via a syringe and needle. Accordingly, in some embodiments, the present disclosure provides a syringe comprising a provided composition. In some embodiments, the syringe is equipped with a needle. In some such embodiments, the needle has a length of ≤1 inch, ≤0.625 inches, or ≤0.5 inches. In some embodiments, the needle has a gauge ranging from 18 G to 26 G, such as 19 G to 25 G, 20 G to 24 G, or 21 G to 23 G. In some embodiments, the needle has a gauge ranging from 16 G to 26 G or from 18 G to 26 G, such as 19 G to 25 G, 20 G to 24 G, or 21 G to 23 G.

In some embodiments, administration of provided compositions is accomplished via a pre-filled syringe or an auto-injector. Accordingly, in some embodiments, the present disclosure provides a pre-filled syringe or an auto-injector comprising a provided composition.

In some embodiments, administration of provided compositions is accomplished via a needle-free injector. Accordingly, in some embodiments, the present disclosure provides a needle-free injector comprising a provided composition.

The present disclosure also provides a vial containing a provided composition.

In some embodiments, provided compositions are administered by a health care professional. In some embodiments, provided compositions are administered by a non-health care professional. In some embodiments, provided compositions are self-administered.

In some embodiments, the present disclosure provides a dosage form comprising a tenofovir agent. In some embodiments, the dosage form further comprises sucrose acetate isobutyrate, a lactic acid-based polymer, and/or a solvent, according to compositions described herein.

In some embodiments, the dosage form is a liquid dosage form. In some embodiments, the liquid dosage form is provided as a solution or a suspension.

In some embodiments, the dosage form is provided in a receptacle selected from a syringe, pump, patch-pump, bolus injector, infusion, auto-injector, or needle-free injector.

The present disclosure also provides dosing regimens for administering provided compositions that are useful in the methods described herein.

Without wishing to be bound by any particular theory, long-acting formulations (as provided herein) allow for less frequent dosing, which can, e.g., increase patient compliance with a dosing regimen. Provided compositions may be particularly useful in patient populations that are prone to non-compliance (e.g., patients who are taking multiple drugs a day and/or who are taking drugs multiple times a day). Additionally or alternatively, provided compositions may be particularly useful for treating and/or preventing diseases or disorders, wherein compliance to a rigid therapeutic regimen is especially beneficial (e.g., combination therapy which relies on the action of multiple agents together). Therefore, in some embodiments, the present disclosure provides methods of increasing subject compliance with a therapeutic regimen comprising a tenofovir agent.

As described above, provided compositions are long-acting formulations and therefore allow for less frequent dosing than other dosage forms of tenofovir agents. Accordingly, in some embodiments, provided compositions are administered once a day, once a week, twice a month, once a month, once every two months, or once every three months.

Under some circumstances, it may be beneficial to administer a loading dose of a tenofovir agent prior to and/or concurrently with provided long-acting formulations, in order to, e.g., achieve a suitable release profile. As used herein, a “loading dose” is one or more doses of an active agent administered in addition to a long-acting formulation. A loading dose may be used to compensate for inadequate plasma levels of the active agent, while a steady state concentration is reached from the long-acting formulation. In some embodiments, methods of administering a provided composition, further comprise administering a loading dose of a tenofovir agent, which may be the same or different as the tenofovir agent in the provided composition. In some such embodiments, the loading dose is administered prior to and/or concurrently with administering a provided composition. In some such embodiments, the loading dose is administered orally or by injection.

In some embodiments, methods of administering a provided composition do not further comprise administering a loading dose.

### Exemplary Embodiments

The following numbered aspects, while non-limiting, are exemplary of certain aspects of the present disclosure:

1. A composition comprising:
(i) an active agent comprising a tenofovir agent; and
(ii) a vehicle comprising a non-polymeric, non-water soluble high viscosity liquid carrier material (HVLCM) having a viscosity of at least 5000 cP at 37° C. that does not crystallize neat at 25° C. and 1 atmosphere.
   2. The composition of aspect 1, wherein the HVLCM is or comprises at least one member selected from sucrose acetate isobutyrate, a stearate ester, propylene glycol, glyceryl, diethylaminoethyl, glycol, a stearate amide, a long-chain fatty acid amide, N,N′-ethylene distearamide, stearamide monoethanolamine (MEA), stearamide diethanolamine (DEA), ethylene bistearamide, cocoamine oxide, a long-chain fatty alcohol, cetyl alcohol, stearyl alcohol, long-chain ester, myristyl myristate, beheny erucate, a glyceryl phosphate, and acetylated sucrose distearate.
   3. The composition of aspect 1 or 2, wherein the HVLCM is or comprises sucrose acetate isobutyrate.
   4. The composition of any one of aspects 1-3, wherein the active agent comprises particles having a median particle size, as measured by laser diffraction, ranging from 0.5 micrometers to 100 micrometers.
   5. The composition of any one of the preceding aspects, wherein the active agent comprises tenofovir alafenamide, or a salt thereof.
   6. The composition of any one of the preceding aspects, wherein the active agent comprises tenofovir alafenamide or a salt thereof having a water solubility of less than or equal to 1 mg/mL.
   7. The composition of any one of the preceding aspects, wherein the tenofovir agent is selected from tenofovir alafenamide hemipamoate, tenofovir alafenamide sebacate, tenofovir alafenamide napsylate, tenofovir alafenamide orotate, tenofovir alafenamide vanillate, and tenofovir alafenamide bis-xinafoate.
   8. The composition of any one of the preceding aspects, wherein the active agent comprises tenofovir alafenamide sebacate.
   9. The composition of any one of the preceding aspects, wherein the composition comprises from about 1 wt % to about 50 wt %, about 2 wt % to about 40 wt %, about 5 wt % to about 30 wt %, about 10 wt % to about 25 wt %, or about 10 wt % to about 20 wt % active agent, based on weight of the vehicle or weight of the composition.
   10. The composition of any one of the preceding aspects, wherein the composition comprises from about 5 wt % to about 95 wt %, about 5 wt % to about 90 wt %, about 10 wt % to about 90 wt %, about 25 wt % to about 80 wt %, about 30 wt % to about 70 wt %, or about 40 wt % to about 60 wt % HVLCM or sucrose acetate isobutyrate, based on weight of the vehicle or weight of the composition.
   11. The composition of any one of the preceding aspects, wherein the composition further comprises a solvent.
   12. The composition of any one of the preceding aspects, wherein the composition further comprises an organic solvent.
   13. The composition of any one of the preceding aspects, wherein the composition further comprises a hydrophilic solvent.
   14. The composition of any one of the preceding aspects, wherein the composition further comprises a hydrophobic solvent.
   15. The composition of aspect 11, wherein the solvent comprises at least one member selected from N-methyl-pyrrolidone (NMP), dimethylsulfoxide (DMSO), propylene carbonate (PC), benzyl alcohol (BA), benzyl benzoate (BB), dimethylacetamide, caprylic/capric triglyceride, polyoxyethylene ester of 12-hydroxystearic acid, ethanol, ethyl lactate, glycofurol, propylene glycol, acetone, methyl acetate, ethyl acetate, methyl ethyl ketone, triacetin, dimethylformamide, tetrahydrofuran, caprolactam, caprolactone, decylmethylsulfoxide, oleic acid, tocopherol, linoleic acid, oleic acid, ricinoleic acid, pyrrolidone, diethyl phthalate, isopropylidene glycerol, and 1-dodecylazacycloheptan-2-one.
   16. The composition of aspect 11, wherein the solvent comprises at least one member selected from N-methyl-pyrrolidone (NMP), dimethylsulfoxide (DMSO), propylene carbonate (PC), benzyl benzoate (BB), dimethylacetamide, caprylic/capric triglyceride, polyoxyethylene ester of 12-hydroxystearic acid, ethanol, ethyl lactate, glycofurol, propylene glycol, acetone, methyl acetate, ethyl acetate, methyl ethyl ketone, triacetin, dimethylformamide, tetrahydrofuran, caprolactam, caprolactone, decylmethylsulfoxide, oleic acid, tocopherol, linoleic acid, oleic acid, ricinoleic acid, pyrrolidone, diethyl phthalate, isopropylidene glycerol, and 1-dodecylazacycloheptan-2-one.
   17. The composition of aspect 11, wherein the solvent comprises at least one of N-methyl-pyrrolidone (NMP), dimethylsulfoxide (DMSO), propylene carbonate (PC), benzyl alcohol (BA), benzyl benzoate (BB), ethanol, and glycofurol.
   18. The composition of aspect 11, wherein the solvent comprises propylene carbonate (PC).
   19. The composition of any one of the preceding aspects, wherein the composition does not comprise N-methyl-pyrrolidone (NMP) and/or does not comprise ethanol.
   20. The composition of any one of the preceding aspects, wherein the composition comprises from about 10 wt % to about 90 wt %, about 10 wt % to about 80 wt %, about 10 wt % to about 60 wt %, about 10 wt % or about 40 wt %, or about 15 wt % to about 35 wt % solvent, based on weight of the vehicle or weight of the composition.
   21. The composition of any one of the preceding aspects, wherein the composition further comprises a polymer.
   22. The composition of aspect 21, wherein the polymer is linear or branched.
   23. The composition of aspects 21 or 22, wherein the polymer comprises a homopolymer.
   24. The composition of any one of aspects 21-23, wherein the polymer comprises a copolymer.
   25. The composition of any one of aspects 21-24, wherein the polymer comprises a lactic acid-based polymer.
   26. The composition of any one of aspects 21-25, wherein the polymer comprises an alkoxy end group, an acid end group, or hydroxyl end group.
   27. The composition of any one of aspects 21-26, wherein the polymer comprises an alkoxy end group that consists of 2 to 24 carbons or 8 to 24 carbons.
   28. The composition of aspect 27, wherein the alkoxy end group consists of 12 carbons.
   29. The composition of any one of aspects 21-28, wherein the polymer is initiated with a member selected from fatty alcohol and diol.
   30. The composition of any one of aspects 21-29, wherein the polymer is initiated with 1,6-hexanediol.
   31. The composition of any one of aspects 21-29, wherein the polymer is initiated with dodecanol.
   32. The composition of any one of aspects 21-31, wherein the polymer comprises poly(lactic acid)(glycolic acid).
   33. The composition of any one of aspects 21-32, wherein the polymer comprises lactic acid repeat units and glycolic acid repeat units in a molar ratio of from about 50:50 to about 100:0, about 70:30 to about 100:0, about 75:25 to about 100:0, or about 85:15 to about 95:5.
   34. The composition of any one of aspects 21-33, wherein the polymer has a weight average molecular weight of less than about 50,000 Daltons, less than about 40,000 Daltons, or less than about 30,000 Daltons, or from about 1000 Daltons to about 50,000 Daltons, about 4000 Daltons to about 40,000 Daltons, about 6000 Daltons to about 30,000 Daltons, about 10,000 Daltons to about 25,000 Daltons, or about 15,000 Daltons to about 20,000 Daltons.
   35. The composition of any one of aspects 21-33, wherein the polymer has a weight average molecular weight of less than about 70,000 Daltons, less than about 60,000 Daltons, less than about 50,000 Daltons, less than about 40,000 Daltons, or less than about 30,000 Daltons, or from about 1000 Daltons to about 50,000 Daltons, about 4000 Daltons to about 40,000 Daltons, about 6000 Daltons to about 30,000 Daltons, about 10,000 Daltons to about 25,000 Daltons, or about 15,000 Daltons to about 20,000 Daltons.
   36. The composition of any one of aspects 21-35, wherein the polymer has a weight average molecular weight after gamma irradiation that is from about 85% to about 99.9%, from about 90% to about 99%, or from about 95% to about 98%, relative to the weight average molecular of the polymer before gamma irradiation.
   37. The composition of any one of aspects 21-36, wherein the composition comprises less than about 40 wt %, less than about 30 wt %, less than about 20 wt %, or less than about 10 wt %, or from about 1 wt % to about 40 wt %, about 2 wt % to about 30 wt %, about 3 wt % to about 20 wt %, or about 5 wt % to about 10 wt % polymer, based on weight of the vehicle or weight of the composition.
   38. The composition of any one of the preceding aspects, wherein the composition does not comprise cellulose acetate butyrate.
   39. The composition of any one of aspects 21-38, wherein the weight ratio of the sucrose acetate isobutyrate to the polymer to the solvent is about 1:0.1-2:0.3-10, or 1:0.2-1:0.4-5, or 1:0.3-0.5:0.5-1.
   40. The composition of any one of the preceding aspects, wherein the vehicle is monophasic when stored at 25° C. for 7 days.
   41. The composition of any one of the preceding aspects, wherein the vehicle is monophasic when stored at 25° C. for 1 month.
   42. The composition of any one of the preceding aspects, wherein the composition has a viscosity of less than 10,000 cP at a shear rate of 100 s⁻¹ at 25° C.
   43. The composition of any one of the preceding aspects, wherein the composition has a viscosity of less than 20,000 cP or less than 10,000 cP at a shear rate of 100 s⁻¹ at 25° C.
   44. The composition of any one of the preceding aspects, wherein the composition has a viscosity of from about 50 cP to about 8000 cP or about 500 cP to about 6000 cP at a shear rate of 150 s⁻¹ at 25° C.
   45. The composition of any one of the preceding aspects, wherein the composition further comprises at least one member selected from viscosity enhancers, antioxidants, preservatives, and particle stabilizers.
   46. The composition of any one of the preceding aspects, wherein the composition comprises from about 0.001 wt % to about 0.35 wt % water, based on total weight of the composition.
   47. The composition of any one of the preceding aspects, wherein when the composition is placed in phosphate buffered saline at 37° C. (e.g., at pH 6.0 or 7.4), the amount of active agent released from the composition after 4 weeks is from about 20% to about 100%, about 20% to about 80%, about 40% to about 100%, about 50% to about 100%, or about 40% to about 80% of the total amount of the active agent in the composition.
   48. The composition of any one of the preceding aspects, wherein when the composition is placed in phosphate buffered saline at 37° C. (e.g., at pH 6.0 or 7.4), the amount of active agent released from the composition after 24 hours is less than about 40%, less than about 30%, less than about 20%, or less than about 10% of the amount released after 28 days.
   49. The composition any one of the preceding aspects, wherein when the composition is placed in phosphate buffered saline at 37° C. (e.g., at pH 6.0 or 7.4), the amount of active agent released after 28 days is greater than about 30%, greater than about 40%, greater than about 50%, greater than about 60%, greater than about 70%, or greater than about 80% of a total amount of active agent in the composition.
   50. The composition any one of the preceding aspects, wherein when the composition is placed in phosphate buffered saline at 37° C. (e.g., at pH 6.0 or 7.4), the amount of active agent released after 28 days is greater than about 20%, greater than about 30%, greater than about 40%, greater than about 50%, greater than about 60%, greater than about 70%, or greater than about 80% of a total amount of active agent in the composition.
   51. The composition of any one of the preceding aspects, wherein the composition has been sterilized.
   52. The composition of any one of the preceding aspects, wherein the composition has been gamma-irradiated.
   53. The composition of aspect 52, wherein the composition has been exposed to an average gamma irradiation dose of less than about 25 kGy.
   54. The composition of aspect 52 or 53, wherein the composition has been exposed to an average gamma irradiation dose from about 15 kGy to about 25 kGy.
   55. The composition of any one of aspects 52-54, wherein, after gamma-irradiation, the composition comprises at least 95%, at least 97%, at least 98%, or at least 99% of the tenofovir agent, relative to the amount of the tenofovir agent before gamma irradiation.
   56. The composition of any one of aspects 52-55, wherein, after gamma-irradiation, the composition comprises no more than about 5%, no more than about 3%, no more than about 2%, or no more than about 1% additional degradation products, relative to the amount of degradation products before gamma irradiation.
   57. The composition of any one of the preceding aspects, wherein the composition does not comprise risperidone.
   58. The composition of any one of the preceding aspects, wherein the composition achieves a therapeutically effective plasma concentration of the active agent, or a metabolite thereof, for at least about 7 days, about 14 days, about 21 days, about 28 days, or more, when the composition is administered subcutaneously as a single dose to a subject.
   59. The composition of any one of the preceding aspects, wherein the composition has been stored.
   60. The composition of any one of the preceding aspects, wherein the composition has been stored at from about 0° C. to about 20° C., about 1° C. to about 10° C., or about 2° C. to about 8° C.
   61. A unit dosage form comprising the composition of any one of the preceding claims.
   62. The unit dosage form of aspect 61, wherein the composition is contained within a vial.
   63. The unit dosage form of aspect 61, wherein the composition is contained within a syringe.
   64. The unit dosage form of aspect 61, wherein the composition is contained within a needle-free injector.
   65. A receptacle containing the composition of any one of aspects 1-60.
   66. A needle-free injector comprising the composition of any one of aspects 1-60.
   67. A composition as defined in any one of aspects 1-60 for use as a medicament.
   68. A composition as defined in any one of aspects 1-60 for use in a method of treating and/or preventing HIV and/or HBV infection.
   69. Use of a composition as defined in any one of aspects 1-60 for the manufacture of a medicament for treating and/or preventing HIV and/or HBV infection.
   70. A process of sterilizing the composition of any one of aspects 1-60, comprising gamma-irradiating the composition.
   71. A method of administering a therapeutically effective dose of a tenofovir agent to a subject in need thereof, the method comprising administering to the subject the composition of any one of aspects 1-60 or the unit dosage form of any one of aspects 61-64.
   72. The method of aspect 71, wherein the administration achieves a plasma tenofovir concentration greater than about 0.01 ng/mL, about 0.1 ng/mL, or about 0.5 ng/mL for at least about 10 days, about 20 days, about 25 days, about 30 days, about 35 days, about 40 days, about 45 days, about 50 days, about 55 days, about 60 days, about 65 days, or longer.
   73. The method of aspect 71 or 72, wherein the administration achieves an intracellular tenofovir diphosphate concentration in peripheral blood mononuclear cells greater than about 10 nM for at least about 10 days, about 20 days, about 25 days, about 30 days, about 35 days, about 40 days, about 45 days, about 50 days, about 55 days, about 60 days, about 65 days, or longer.
   74. The method of any one of aspects 71-73, wherein the composition or unit dosage form has been established to achieve a plasma tenofovir concentration greater than about 0.01 ng/mL, about 0.1 ng/mL, or about 0.5 ng/mL for at least about 10 days, about 20 days, about 25 days, about 30 days, about 35 days, about 40 days, about 45 days, about 50 days, about 55 days, about 60 days, about 65 days, or longer in a dog subject.
   75. The method of any one of aspects 71-74, wherein the composition or unit dosage form has been established to achieve an intracellular tenofovir diphosphate concentration in peripheral blood mononuclear cells greater than about 10 nM for at least about 10 days, about 20 days, about 25 days, about 30 days, about 35 days, about 40 days, about 45 days, about 50 days, about 55 days, about 60 days, about 65 days, or longer in a dog subject.
   76. The method of any one of aspects 71-75, wherein the administering comprises administering the composition or unit dosage form subcutaneously.
   77. The method of any one of aspects 71-76, wherein the composition is self-administered.
   78. The method of any one of aspects 71-77, wherein the composition is administered by a non-health care professional.
   79. The method of any one of aspects 71-78, wherein the composition is administered with a needle and syringe.
   80. The method of aspect 79, wherein the needle has a length of less than or equal to 1 inch.
   81. The method of aspect 79, wherein the needle has a length of less than or equal to ⅝ inch.
   82. The method of aspect 79, wherein the needle has a length of less than or equal to 0.5 inch.
   83. The method of any one of aspects 71-82, wherein the composition is administered with a pre-filled syringe or an auto-injector.
   84. The method of any one of aspects 71-83, wherein the composition is administered once a month.
   85. The method of any one of aspects 71-84, wherein the subject is receiving or has received an additional therapeutic agent.
   86. A method of treating and/or preventing HIV infection, the method comprising administering the composition of any one of aspects 1-60 or the unit dosage form of any one of aspects 61-64.
   87. A method of treating and/or preventing HBV infection, the method comprising administering the composition of any one of aspects 1-60 or the unit dosage form of any one of aspects 61-64.
   88. A method of manufacturing the composition of any one of the preceding aspects, comprising:
(a) providing the tenofovir agent; and
(b) combining the tenofovir agent with the vehicle to form the composition.
   89. The method of aspect 88, further comprising reducing the amount of water in the composition.
   90. The method of aspect 89, wherein the reducing the amount of water in the composition comprises placing the mixture under an inert gas, such as nitrogen.
   91. The method of any one of aspects 88-90, further comprising heating the mixture.
   92. The method of any one of aspects 88-97, further comprising mixing the mixture.

### EXAMPLES

### Abbreviations

ACN acetonitrile
BB benzyl benzoate
DCM dichloromethane
DD dodecanol
DMSO dimethylsulfoxide
EtOH ethanol
ND not determined
NMP N-methyl-2-pyrrolidone
PC propylene carbonate
PLA poly(lactic acid)
PLA-0 PLA with C₆-C₁₂ aliphatic chain ester end group (MW: <20 kDa)
PLA-1 DL-PLA lactic acid terminated (MW: 14 kDa)
PLA-2 DL-PLA lactic acid terminated (MW: 16 kDa)
PLA-3 DL-PLA initiated with 1-dodecanol (MW: 16 kDa)
PLA-4 DL-PLA (MW: 13 kDa)
PLGA poly(lactic acid)(glycolic acid)
50-50 PLGA-DD 50:50 DL-PLGA initiated with 1-dodecanol (MW: 7 kDa)
50-50 PLGA-LA 50:50 DL-PLGA lactic acid terminated (MW: 6 kDa)
65-35 PLGA-DD 65:35 DL-PLGA initiated with 1-dodecanol (MW: 7-8 kDa)
65-35 PLGA-2 65:35 PLGA initiated with 1-dodecanol (MW: 48.4 kDa)
75-25 PLGA 75:25 DL-PLGA initiated with 1-dodecanol (MW: 8 kDa)
75-25 PLGA-2 75:25 PLGA initiated with 1-dodecanol (MW: 40 kDa)
75-25 PLGA-3 75:25 PLGA initiated with 1-dodecanol (MW: 51 kDa)
75-25 PLGA-4 75:25 PLGA initiated with 1-dodecanol (MW: 29 kDa)
85-15 PLGA 85:15 PLGA terminated with hydroxyacid (MW: 40-80 kDa)
90-10 PLGA-1 90:10 DL-PLGA initiated with 1-dodecanol (MW: 18 kDa)
90-10 PLGA-2 90:10 DL-PLGA initiated with 1-dodecanol (MW: 8 kDa)
90-10 PLGA-3 90:10 DL-PLGA initiated with 1-dodecanol (MW: 11 kDa)
SAIB sucrose acetate isobutyrate
TAF tenofovir alafenamide
TFV-DP tenofovir diphosphate

### Example 1. Preparation of Vehicle Compositions

A representative method of making a formulation comprising SAIB, lactic acid-based polymer, and solvent follows:

SAIB was heated in a 60° C. oven. Solvent was weighed into a container with a stir bar. Lactic acid-based polymer(s) were added to the solvent with stirring until dissolution was achieved. Heated SAIB was added and stirred until a homogeneous composition was obtained. In some cases, the resulting vehicle was filtered through a flat sheet filter with a pore size of 5 microns.

Another representative method of making a formulation comprising SAIB, lactic acid-based polymer, and solvent follows:

Poly(lactic acid)(glycolic acid) (PLGA) was removed from cold storage and allowed to warm to room temperature. The polymer was weighed in a glass jar. Next, propylene carbonate (PC) was dispensed into the glass jar. To dissolve the PLGA in the PC, the mixture was placed in a rotator and rotated at 20 rpm at room temperature for about 12 hours. SAIB was heated to 80° C. for approximately an hour. The heated SAIB was poured into the glass jar containing the PLGA and PC. The mixture was rotated in an oven at 50° C. at 20 rpm for about 2 hours. The jar was removed from the oven and allowed to cool to room temperature.

Vehicle compositions were prepared according to Table 1A and Table 1B below. The viscosities of the vehicles were measured at a shear rate of 100 s⁻¹ to 500 s⁻¹ at 25° C. Unless noted otherwise, the vehicles remained as a single phase when maintained at 25° C. for a one week period.

TABLE 1A

| Vehicle | | Vehicle Viscosity |
|---|---|---|
| No. | Vehicle (w/w/w %) | (25° C., cP) |
| V1 | SAIB/NMP/85-15 PLGA | |
| | (65/25/10) | |
| V2 | SAIB/EtOH/PLA-0 | |
| | (65/25/10) | |
| V3 | SAIB/NMP/75-25 PLGA | 411 |
| | (50/30/20) | |
| V4 | SAIB/DMSO/75-25 PLGA | 435 |
| | (48/32/20) | |
| V5 | SAIB/PC/75-25 PLGA | 384 |
| | (44/37/19) | |
| V6 | SAIB/BB/75-25 PLGA | 255 |
| | (30/55/15) | |
| V7 | SAIB/BB/PLA-1 | 186 |
| | (15/70/15) | |
| V8 | SAIB/PC/PLA-3 | 260 |
| | (33/47/20) | |
| V9 | SAIB/PC/65-35 PLGA-DD | 332 |
| | (44/37/19) | |
| V10 | SAIB/NMP/BB/90-10 PLGA-1 | 218 |
| | (35/25/25/15) | |
| V11 | SAIB/PC/90-10 PLGA-1 | 252 |
| | (40/45/15) | |
| V12 | SAIB/BB/65-35 PLGA-DD | 115 |
| | (45/50/5) | |
| V13 | SAIB/PC/BB/90-10 PLGA-1 | 226 |
| | (30/27.5/27.5/15) | |
| V14 | SAIB/PC/BB/90-10 PLGA-1 | 170 |
| | (40/25/25/10) | |
| V15 | SAIB/BB/90-10 PLGA-1 | 446 |
| | (10/70/20) | |
| V16 | SAIB/PC/75-25 PLGA | 384 |
| | (44/37/19) | |
| V17 | SAIB/BB/PLA-1 | 186 |
| | (15/70/15) | |
| V18 | SAIB/BB/PLA-1 | 186 |
| | (15/70/15) | |
| V19 | SAIB/BB/90-10 PLGA-1 | 446 |
| | (10/70/20) | |
| V22 | SAIB/NMP/75-25 PLGA | 411 |
| | (50/30/20) | |
| V23 | SAIB/DMSO/75-25 PLGA | 435 |
| | (48/32/20) | |
| V24 | SAIB/NMP/75-25 PLGA | 411 |
| | (50/30/20) | |
| V25 | SAIB/DMSO/75-25 PLGA | 435 |
| | (48/32/20) | |
| N26 | SAIB/PC/75-25 PLGA | 384 |
| | (44/37/19) | |
| N27 | SAIB/PC/75-25 PLGA | 1435 |
| | (53/28/19) | |
| V28 | SAIB/PC/75-25 PLGA | 1004 |
| | (33/37/30) | |
| V29 | SAIB/PC/90-10 PLGA | 995 |
| | (44/37/19) | |
| V30 | SAIB/PC/75-25 PLGA | 384 |
| | (44/37/19) | |
| V31 | SAIB/DMSO/75-25 PLGA | 1212 |
| | (55/25/20) | |
| V32 | SAIB/DMSO/75-25 PLGA | 962 |
| | (38/32/30) | |
| V33 | SAIB/DMSO/PLA-3 | 789 |
| | (48/32/20) | |
| V34 | SAIB/DMSO/75-25 PLGA | 435 |
| | (48/32/20) | |
| V35 | SAIB/BB/PLA-2 | 1664 |
| | (43/42/15) | |
| V36 | SAIB/NMP/75-25 PLGA | 1365 |
| | (57/23/20) | |
| V37 | SAIB/BB/PLA-3 | 1331 |
| | (43/42/15) | |
| V38 | SAIB/BB/90-10 PLGA-1 | 1931 |
| | (30/50/20) | |
| V39 | SAIB/BB/DMSO/90-10 PLGA-1 | 1452 |
| | (34/40/6/20) | |
| V40 | SAIB/NMP/90-10 PLGA-1 | 1032 |
| | (50:30:20) | |
| V41 | SAIB/PC/PLA-2 | 907 |
| | (44/37/19) | |
| V42 | SAIB/PC/65-35 PLGA-DD | 1221 |
| | (53/28/19) | |
| V43 | SAIB/PC/75-25 PLGA | 639 |
| | (62/28/10) | |
| V44 | SAIB/PC/75-25 PLGA | 583 |
| | (53/32/15) | |
| V45 | SAIB/PC/BB/75-25 PLGA | 403 |
| | (43.5/37/0.5/19) | |
| V46 | SAIB/PC/BB/75-25 PLGA | 388 |
| | (43.5/36/1.5/19) | |
| V47 | SAIB/PC/BB/75-25 PLGA | 422 |
| | (43/35/3/19) | |
| V48 | SAIB/PC/IPM/75-25 PLGA | 374 |
| | (43/36/2/19) | |
| V49 | SAIB/PC/50-50 PLGA-DD/PLA-2 | 787 |
| | (44/37/5/14) | |
| V50 | SAIB/PC/50-50 PLGA-DD | |
| | (53/28/19) | |
| V51 | SAIB/PC/50-50 PLGA-DD | 427 |
| | (44/37/19) | |
| V52 | SAIB/PC/90-10 PLGA-2 | 1176 |
| | (53/28/19) | |
| V53 | SAIB/PC/50-50 PLGA-LA | |
| | (43/37/19) | |
| V54 | SAIB/PC/PLA-2/50-50 PLGA-LA | 911 |
| | (44/37/17.5/1.5) | |
| V55 | SAIB/PC/PLA/50-50 PLGA-LA | 777 |
| | (44/37/14/5) | |
| V56 | SAIB/PC/75-25 PLGA/50-50 PLGA-LA | 441 |
| | (44/37/17.5/1.5) | |
| V57 | SAIB/PC/75-25 PLGA/50-50 PLGA-LA | 471 |
| | (44/37/14/5) | |
| V58 | SAIB/PC/90-10 PLGA-1 | 1092 |
| | (62/28/10) | |
| V59 | SAIB/PC/PLA-3 | 871 |
| | (44/37/19) | |
| V60 | SAIB/PC/75-25 PLGA | 384 |
| | (44/37/19) | |
| V61 | SAIB/PC/90-10 PLGA-1 | 995 |
| | (44/37/19) | |
| V62 | SAIB/PC/90-10 PLGA-1 | 995 |
| | (44/37/19) | |
| V63 | SAIB/PC/PLA-3 | 272 |
| | (32/49/19) | |
| V64 | SAIB/PC/BB/PLA-3 | 265 |
| | (31.5/48/1.5/19) | |
| V65 | SAIB/PC/90-10 PLGA-1 | 1092 |
| | (62/28/10) | |
| V66 | SAIB/PC/PLA-3 | 101 |
| | (41/49/10) | |
| V67 | SAIB/PC/PLA-3 | 61 |
| | (23/58/19) | |
| V68 | SAIB/PC/PLA-3 | 61 |
| | (23/58/19) | |
| V69 | SAIB/PC/90-10 PLGA-1 | 974 |
| | (52/33/15) | |
| V70 | SAIB/PC/90-10 PLGA-1 | 958 |
| | (71/24/5) | |
| V71 | SAIB/PC/90-10 PLGA-1 | 753 |
| | (60/30/10) | |
| V72 | SAIB/PC/90-10 PLGA-1 | 995 |
| | (44/37/19) | |
| V73 | SAIB/PC | 959 |
| | (79/21) | |
| V74 | SAIB/PC/NMP/90-10 PLGA-1 | 928 |
| | (46/27/8/19) | |
| V75 | SAIB/PC/NMP/90-10 PLGA-1 | 734 |
| | (61.5/22/6.5/10) | |
| V76 | SAIB/PC/DMSO/90-10 PLGA-1 | 727 |
| | (61/22/7/10) | |
| V77 | SAIB/PC/90-10 PLGA-1 | 995 |
| | (44/37/19) | |
| V78 | SAIB/PC/90-10 PLGA-1 | 753 |
| | (60/30/10) | |
| V79 | SAIB/PC/PLA-3 | 728 |
| | (44/37/19) | |
| V80 | SAIB/PC/PLA-3 | 656 |
| | (60/30/10) | |
| V81 | SAIB/PC/PLA-3 | 728 |
| | (44/37/19) | |
| V82 | SAIB/PC/PLA-3 | 728 |
| | (44/37/19) | |
| V83 | SAIB/PC/90-10 PLGA-l/PLA-3 | 707 |
| | (60/30/5/5) | |
| V84 | SAIB/PC/90-10 PLGA-1 | 974 |
| | (52/33/15) | |
| V85 | SAIB/PC/90-10 PLGA-1 | 958 |
| | (71/24/5) | |
| V90 | SAIB/NMP/75-25 PLGA | 403 |
| | (50/30/20) | |
| V91 | SAIB/PC/75-25 PLGA | 412 |
| | (44/37/19) | |
| V92 | SAIB/BB/75-25 PLGA | 1623 |
| | (40/40/20) | |
| V93 | SAIB/DMSO/75-25 PLGA | 429 |
| | (48/32/20) | |
| V94 | SAIB/BB/PLA-4 | 639 |
| | (20/60/20) | |
| V95 | SAIB/NMP/75-25 PLGA | 34 |
| | (20/60/20) | |
| V96 | SAIB/PC/PLGA (20/60/20) | 73 |
| | (75:25 PLGA) | |
| V97 | SAIB/BB/75-25 PLGA | 318 |
| | (20/60/20) | |
| V98 | SAIB/DMSO/PLGA (20/60/20) | 40 |
| | (75:25 PLGA) | |
| V99 | SAIB/BA/EtOH/75-25 PLGA | 15 |
| | (20/30/20/20) | |
| V100 | SAIB/NMP/65-35 PLGA | 409 |
| | (50/30/20) | |
| V101 | SAIB/NMP/90-10 PLGA-3 | 585 |
| | (50/30/20) | |
| V102 | SAIB/GF/75-25 PLGA | 1141 |
| | (44/37/19) | |
| V103 | SAIB/GF/75-25 PLGA | 270 |
| | (20/60/20) | |
| V104 | SAIB/NMP/90-10 PLGA-2 | |
| | (50/30/20) | |
| V105 | SAIB/PC/90-10 PLGA-2 | |
| | (44/37/19) | |
| V106 | SAIB/NMP/75-25 PLGA | |
| | (50/30/20) | |
| V107 | SAIB/PC/75-25 PLGA | |
| | (44/37/19) | |
| V108 | SAIB/DMSO/PLGA | |
| | (48/32/20) | |
| V109 | SAIB/BB/PLA-4 | |
| | (20/60/20) | |
| V110 | SAIB/DMSO/75-25 PLGA | |
| | (20/60/20) | |
| V111 | SAIB/NMP/90-10 PLGA-3 | |
| | (50/30/20) | |

TABLE 1B

| Vehicle | | Vehicle Viscosity |
|---|---|---|
| No. | Vehicle (w/w/w %) | (25° C., cP) |
| V112 | SAIB/PC/65-35 PLGA-2 | 1867 |
| | (25/55/20) | |
| V113 | SAIB/PC/90-10 PLGA-1 | 1882 |
| | (38/37/25) | |
| V114 | SAIB/PC/DMSO/90-10 PLGA-1 | 1990 |
| | (50/21/9/20) | |
| V115 | SAIB/PC/EtOH/90-10 PLGA-1 | 1530 |
| | (53/21.5/5.5/20) | |
| V116 | SAIB/PC/75-25 PLGA-2 | 2108 |
| | (34/46/20) | |
| V117 | SAIB/PC/75-25 PLGA-3 | 1951 |
| | (25/55/20) | |
| V118 | SAIB/PC/75-25 PLGA-4 | 1780 |
| | (37/43/20) | |

### Example 2. Synthesis of Tenofovir Alafenamide

Methods of synthesizing tenofovir alafenamide were described in PCT Publication No. WO 02/08241 and U.S. Pat. No. 8,664,386, each of which is hereby incorporated by reference in its entirety.

Methods for synthesizing salts and solid forms of tenofovir alafenamide were described in WO 2013/025788, WO 2016/205141, and WO 2018/144390, all of which are hereby incorporated by reference in their entirety. For example, crystalline tenofovir alafenamide sebacate can be prepared according to the following procedure: Tenofovir alafenamide (about 1 g) was mixed with sebacic acid (about 0.4 g) and acetone (about 10 mL). The solution was put in a glass vial with an open lid and allowed to evaporate to give tenofovir alafenamide sebacate Form I.

Tenofovir alafenamide sebacate Form I was characterized by X-ray powder diffraction (XRPD). XRPD patterns were collected on a PANanalytical XPERT-PRO diffractometer at ambient conditions under the following experimental settings: 45 KV, 40 mA, Kα1=1.5406 Å, scan range 2 to 40°, step size 0.0084 or 0.0167°, measurement time: 5 min. The XRPD pattern of tenofovir alafenamide sebacate Form I is shown in FIG. 1 and summarized in Table 2.

TABLE 2

| Peak | Relative |
|---|---|
| Position | Intensity |
| [°2θ] | [%] |
| 5.3 | 14 |
| 6.6 | 100 |
| 9.4 | 70 |
| 9.6 | 76 |
| 10.5 | 5 |
| 11.7 | 29 |
| 12.6 | 10 |
| 14.0 | 6 |
| 14.8 | 41 |
| 15.7 | 38 |
| 16.9 | 9 |
| 18.7 | 51 |
| 19.3 | 44 |
| 19.8 | 57 |
| 20.9 | 11 |
| 21.6 | 7 |
| 22.1 | 38 |
| 22.9 | 7 |
| 23.4 | 22 |
| 23.8 | 22 |
| 25.3 | 7 |
| 26.2 | 18 |
| 26.5 | 9 |
| 27.4 | 7 |
| 28.2 | 12 |
| 28.7 | 5 |
| 29.0 | 11 |
| 33.3 | 6 |
| 37.9 | 6 |

Tenofovir alafenamide sebacate Form I was also characterized by differential scanning calorimetry (DSC). DSC thermograms were collected on a TA Instruments Q2000 system equipped with a 50 position auto-sampler. The calibration for energy and temperature was carried out using certified indium. Typically 1-5 mg of each sample, in a pin-holed aluminum pan, was heated at 10° C./min from 25° C. to 300° C. A purge of dry nitrogen at 50 mL/min was maintained over the sample throughout the measurement. The DSC thermogram of tenofovir alafenamide sebacate Form I is shown in FIG. 2. Tenofovir alafenamide sebacate has a solubility of approx. 0.7 mg/mL in water at 22° C.

### Example 3. Solubility of Tenofovir Agents

The release of an active agent from SAIB/polymer/solvent formulations (e.g., provided vehicle formulations) depends on a variety of factors, including the solubility of the active agent in the solvent of provided formulations. This Example demonstrates the solubility of TAF free base and in salt forms with various solvents.

Briefly, samples were prepared by mixing active agent and solvent and were tested at various time points after mixing and after storage at various temperatures. The samples were tested for the solubility of the active agent (mg/mL), calculated as the free base equivalent. The results and compositions tested are shown in Table 3. Each data point is the average of two replicates unless otherwise specified. The data points with an asterisk (*) were taken at 4 days, as opposed to 5 days.

TABLE 3

| | Solubility (mg/mL, Calculated as Free Base Equivalent) | | | | |
|---|---|---|---|---|---|
| | | | TAF | TAF | |
| | | | Hemi- | Hemi- | TAF |
| | TAF Free base | | fumarate | pamoate | Sebacate |
| | 37° C./ | 37° C./ | 37° C./ | 37° C./ | 37° C./ |
| Solvent | 1 day | 5 days | 5 days | 5 days | 5 days |
| DCM | 146 | 384 | 97 | 220 | 216 |
| DMSO | 96 | 182 | 132 | 203 | 109 |
| NMP | 96 | 161 | 160 | 93 | 88 |
| ACN | 15 | 13 | 11 | 8 | 3 |
| PC | 15 | 26 | 22 | 46 | 6 |
| BB | Not | 5.0 | 3.9* | 1.5* | 2.2* |
| | determined | (n = 1)* | | | |

The present disclosure encompasses the recognition that solvents can be chosen in order to achieve a desirable release rate from the formulation. In some embodiments, a solvent is chosen in which the active agent (e.g., tenofovir agent) is less soluble in order to provide a slower release rate from the formulation. In some embodiments, a solvent is chosen in which the active agent (e.g., tenofovir agent) is more soluble in order to provide a faster release rate from the formulation.

### Example 4. Preparation of Provided Compositions

Provided compositions were prepared according to the following general procedure: Tenofovir agent was added to a vehicle composition (prepared as described in Example 1), followed by homogenization. Before being combined with the vehicle, the tenofovir agent typically had a d90 particle size of about 20 to 30 microns, except for formulation F59 which used TAF sebacate milled to a d90 particle size of about 3 microns.

The formulations described in Table 4A and Table 4B were prepared according to the above procedure. In Table 4A and Table 4B, % active loading (w/w) is based on TAF free base equivalence values. The viscosities of the formulations were measured at a shear rate of 100 s⁻¹ to 500 s⁻¹ at 25° C.

TABLE 4A

| | | % | | | |
|---|---|---|---|---|---|
| | | Active | | | Form. |
| Form. | | Loading | Solution or | | Viscosity |
| No. | Active Agent | (w/w) | Suspension | Vehicle | (25° C., cP) |
| F1 | TAF sebacate | 8.6 | Suspension | V1 | |
| F2 | TAF free base | 8.6 | Solution | V2 | |
| F3 | TAF free base | 8.6 | Suspension | V1 | |
| F4 | TAF free base | 7.8 | Suspension | V3 | 618 |
| F5 | TAF free base | 7.8 | Solution | V4 | 725 |
| F6 | TAF free base | 7.8 | Suspension | V5 | 553 |
| F7 | TAF free base | 7.8 | Suspension | V6 | 371 |
| F8 | TAF free base | 7.8 | Suspension | V7 | 292 |
| F9 | TAF free base | 7.8 | Suspension | V8 | 390 |
| F10 | TAF free base | 7.8 | Suspension | V9 | 503 |
| F11 | TAF free base | 7.8 | Suspension | V10 | 316 |
| F12 | TAF free base | 7.8 | Suspension | V11 | 367 |
| F13 | TAF free base | 7.8 | Suspension | V12 | 196 |
| F14 | TAF free base | 7.8 | Suspension | V13 | 345 |
| F15 | TAF free base | 7.8 | Suspension | V14 | 262 |
| F16 | TAF free base | 7.8 | Suspension | V15 | 630 |
| F17 | TAF sebacate | 7.8 | Suspension | V16 | 793 |
| F18 | TAF sebacate | 7.8 | Suspension | V17 | 492 |
| F19 | TAF hemipamoate | 7.8 | Suspension | V18 | 301 |
| F20 | TAF hemipamoate | 7.8 | Suspension | V19 | 673 |
| F23 | TAF sebacate | 7.8 | Suspension | V22 | |
| F24 | TAF sebacate | 7.8 | Solution | V23 | 758 |
| F25 | TAF hemipamoate | 7.8 | Solution | V24 | |
| F26 | TAF hemipamoate | 7.8 | Solution | V25 | |
| F27 | TAF hemipamoate | 7.8 | Suspension | V26 | |
| F28 | TAF sebacate | 7.8 | Suspension | V27 | 2609 |
| F29 | TAF sebacate | 7.8 | Suspension | V28 | 2070 |
| F30 | TAF sebacate | 7.8 | Suspension | V29 | 2063 |
| F31 | TAF sebacate | 15.6 | Suspension | V30 | 2317 |
| F32 | TAF free base | 7.8 | Suspension | V31 | 2036 |
| F33 | TAF free base | 7.8 | Solution | V32 | 1516 |
| F34 | TAF free base | 7.8 | Solution | V33 | 1344 |
| F35 | TAF free base | 15.6 | Suspension | V34 | 1126 |
| F36 | TAF hemipamoate | 7.8 | Suspension | V35 | 2445 |
| F37 | TAF free base | 7.8 | Suspension | V36 | 2123 |
| F38 | TAF hemipamoate | 7.8 | Suspension | V37 | 1940 |
| F39 | TAF hemipamoate | 7.8 | Suspension | V38 | 2770 |
| F40 | TAF hemipamoate | 7.8 | Suspension | V39 | 3358 |
| F41 | TAF free base | 7.8 | Suspension | V40 | 1626 |
| F42 | TAF sebacate | 7.8 | Suspension | V41 | 1912 |
| F43 | TAF sebacate | 7.8 | Suspension | V42 | 2304 |
| F44 | TAF sebacate | 7.8 | Suspension | V43 | 1283 |
| F45 | TAF sebacate | 7.8 | Suspension | V44 | 1222 |
| F46 | TAF sebacate | 7.8 | Suspension | V45 | 833 |
| F47 | TAF sebacate | 7.8 | Suspension | V46 | 853 |
| F48 | TAF sebacate | 7.8 | Suspension | V47 | 895 |
| F49 | TAF sebacate | 7.8 | Suspension | V48 | 775 |
| F50 | TAF sebacate | 7.8 | Suspension | V49 | 1478 |
| F51 | TAF sebacate | 7.8 | Suspension | V51 | 840 |
| F52 | TAF sebacate | 7.8 | Suspension | V52 | 2250 |
| F53 | TAF sebacate | 7.8 | Suspension | V53 | 743 |
| F54 | TAF sebacate | 7.8 | Suspension | V54 | 1687 |
| F55 | TAF sebacate | 7.8 | Suspension | V55 | 1568 |
| F56 | TAF sebacate | 7.8 | Suspension | V56 | 879 |
| F57 | TAF sebacate | 7.8 | Suspension | V57 | 937 |
| F58 | TAF sebacate | 7.8 | Suspension | V58 | 2280 |
| F59^{b} | TAF sebacate | 7.8 | Suspension | V58 | |
| F60 | TAF sebacate | 7.8 | Suspension | V59 | 1610 |
| F61 | TAF sebacate | 23.4 | Suspension | V60 | |
| F62 | TAF sebacate | 15.6 | Suspension | V61 | 4694 |
| F63 | TAF sebacate | 23.4 | Suspension | V62 | |
| F64 | TAF sebacate | 23.4 | | V63 | 14900 |
| F65 | TAF sebacate | 23.4 | | V64 | 16300 |
| F66 | TAF sebacate | 15.6 | Suspension | V65 | 5137 |
| F67 | TAF sebacate | 15.6 | | V66 | 13800 |
| F68 | TAF sebacate | 23.4 | | V67 | 14300 |
| F69 | TAF hemipamoate | 23.4 | NA | V68 | |
| F70 | TAF sebacate | 15.6 | Suspension | V69 | 3784 |
| F71 | TAF sebacate | 15.6 | Suspension | V70 | 4113 |
| F72 | TAF sebacate | 19.5 | Suspension | V71 | 5679 |
| F73 | TAF sebacate | 19.5 | Suspension | V72 | 6739 |
| F74 | TAF sebacate | 19.5 | Suspension | V73 | 7672 |
| F75 | TAF sebacate | 19.5 | Suspension | V74 | 4204 |
| F76 | TAF sebacate | 19.5 | Suspension | V75 | 3758 |
| F77 | TAF sebacate | 19.5 | Suspension | V76 | 3251 |
| F78 | TAF sebacate | 23.4 | Suspension | V77 | 7185 |
| F79 | TAF sebacate | 23.4 | Suspension | V78 | 5778 |
| F80 | TAF sebacate | 23.4 | Suspension | V79 | 2644 |
| F81 | TAF sebacate | 23.4 | Suspension | V80 | 4745 |
| F82 | TAF sebacate | 27.3 | Suspension | V81 | 8544 |
| F83 | TAF sebacate | 31.2 | | V82 | |
| F84 | TAF sebacate | 23.4 | Suspension | V83 | 5251 |
| F85 | TAF sebacate | 23.4 | Suspension | V84 | 6427 |
| F86 | TAF sebacate | 23.4 | Suspension | V85 | 6596 |
| F87 | TAF sebacate | 11.7 | Suspension | V86 | 1930 |

^{a}Phase separation observed.
^{b}Active agent was milled to a d90 particle size of about 3 microns prior to combining with vehicle.

TABLE 4B

| | | % | | | |
|---|---|---|---|---|---|
| | | Active | | | Form. |
| Form. | | Loading | Solution or | | Viscosity |
| No. | Active Agent | (w/w) | Suspension | Vehicle | (25° C., cP) |
| F88 | TAF sebacate | 7.8 | Suspension | V112 | |
| F89 | TAF sebacate | 7.8 | Suspension | V113 | |
| F90 | TAF sebacate | 7.8 | Suspension | V114 | |
| F91 | TAF sebacate | 7.8 | Suspension | V115 | |
| F92 | TAF sebacate | 7.8 | Suspension | V116 | 3009 |
| F93 | TAF sebacate | 7.8 | Suspension | V117 | 2639 |
| F94 | TAF sebacate | 7.8 | Suspension | V118 | 2178 |

### Example 5. Water Content of Provided Compositions

A vehicle composition including 62/28/10 (wt %) SAIB/PC/PLGA-2 was prepared as described in Example 4 above, and a formulation was prepared by using that vehicle in a formulation including 7.8 wt % TAF sebacate (calculated as the free base equivalent).

The vehicle was initially treated under 2 different conditions: (1) 5 g of the vehicle was transferred into 20 mL scintillation vials and the lids were removed. Half of the vials were left on a laboratory bench at ambient temperature, and half were stored in a reference standard chamber containing dessicator. (2) 20 g of the vehicle was transferred into a 60 mL jar. The vehicle was stirred in a 40° C. oven using stir bar and magnetic stirrers. Samples were taken at various timepoints and tested for water content before and after each treatment (Table 5).

TABLE 5

| | % Water | | | | | |
|---|---|---|---|---|---|---|
| | | | | Average | | |
| Sample | n = 1 | n = 1 | n = 1 | (n = 3) | Std dev | % RSD |
| T = 0 | 0.21 | 0.21 | 0.21 | 0.21 | 0.00 | 0.60 |
| Ambient, 48 hrs | 0.24 | 0.25 | 0.24 | 0.24 | 0.00 | 0.69 |
| Ambient with | 0.31 | 0.31 | 0.31 | 0.31 | 0.00 | 0.38 |
| Desiccators, 48 | | | | | | |
| hrs | | | | | | |
| Ambient, 96 hrs | 0.32 | 0.32 | 0.33 | 0.32 | 0.01 | 1.66 |
| Ambient with | 0.39 | 0.38 | 0.38 | 0.38 | 0.00 | 1.00 |
| Desiccators, 96 | | | | | | |
| hrs | | | | | | |
| 40° C., 8 hrs | 0.10 | 0.10 | 0.11 | 0.10 | 0.00 | 2.95 |
| 40° C., | 0.13 | 0.13 | 0.14 | 0.13 | 0.00 | 2.96 |
| Overnight | | | | | | |

Containers of the vehicle alone and formulation comprising active agent were placed into a glove box containing nitrogen and stirred overnight using stirs bars and magnetic stirrers. The vehicle and formulation comprising active agent were tested for water content both before and after exposure to nitrogen in the glove box, and samples were taken at various timepoints during the nitrogen exposure. Tables 6 and 7 below show the results for the vehicle and formulation, respectively.

TABLE 6

| | % Water | | | | |
|---|---|---|---|---|---|
| Sample | | | Average | | |
| (vehicle) | n = 1 | n = 1 | (n = 2) | Std dev | % RSD |
| T = 0 | 0.21 | 0.23 | 0.22 | 0.02 | 8.08 |
| T = 2 hrs | 0.20 | 0.19 | 0.19 | 0.00 | 1.97 |
| T = 6 hrs | 0.14 | 0.13 | 0.13 | 0.01 | 8.06 |
| T = 19 hrs | 0.06 | 0.04 | 0.05 | 0.01 | 16.20 |
| T = 25 hrs | 0.04 | 0.02 | 0.03 | 0.01 | 30.08 |

TABLE 7

% Water

| Sample | | | Average | | |
|---|---|---|---|---|---|
| (formulation) | n = 1 | n = 1 | (n = 2) | Std dev | % RSD |
| T = 0 | 0.23 | 0.23 | 0.23 | 0.00 | 0.75 |
| T = 2 hrs | 0.19 | 0.20 | 0.19 | 0.00 | 1.02 |
| T = 6 hrs | 0.12 | 0.13 | 0.13 | 0.00 | 1.68 |
| T = 19 hrs | 0.04 | 0.05 | 0.04 | 0.00 | 8.13 |
| T = 25 hrs | 0.02 | 0.03 | 0.03 | 0.00 | 12.78 |

As can be seen from Tables 6 and 7, exposure to nitrogen surprisingly reduced the water content from 0.22% to 0.03% (vehicle alone), and from 0.23% to 0.03% (formulation).

### Example 6. Irradiation Effect

Injectable formulations are often irradiated to render them aseptic for use. This Example demonstrates the stability of the formulations following irradiation.

Representative samples were prepared as generally described in Example 4 above with components as summarized in Table 8. Samples were gamma irradiated at 15-20 kGy, and the samples were tested at various time points after irradiation and after storage at various temperatures. The samples were tested for the concentration of active agent in the sample. The concentration of active agent in the formulation was essentially unchanged by irradiation after storage under various conditions.

TABLE 8

| | | 2 wk, | 4 wk, | 4 wk, | |
|---|---|---|---|---|---|
| | t = 0 | 37° C. | 37° C. | 25° C. | Post |
| | Conc | Conc | Conc | Conc | irradiation |
| Form. | (mg/g) | (mg/g) | (mg/g) | (mg/g) | Conc (mg/g) |
| No. | Avg | Avg | Avg | Avg | Avg |
| F4 | 80.7 | 74.2 | 68.4 | 77.1 | 82.8 |
| F5 | 80.0 | 74.6 | 67.3 | 76.3 | 77.7 |
| F6 | 80.8 | 77.3 | 75.6 | 79.3 | 79.1 |
| F7 | 80.5 | 77.2 | 76.1 | 73.9 | 78.4 |
| F8 | 81.4 | 75.8 | 73.3 | 77.8 | 78.0 |
| F9 | 80.6 | 76.6 | 74.3 | 78.5 | 77.7 |
| F10 | 80.5 | 76.4 | 73.2 | 78.8 | 78.0 |
| F11 | 80.4 | 73.8 | 66.7 | | 78.5 |
| F12 | 80.4 | 77.7 | 75.9 | | 78.8 |
| F13 | 81.8 | 77.5 | 77.3 | 78.6 | 78.5 |
| F14 | 79.5 | 77.9 | 76.3 | | 78.4 |
| F15 | 79.9 | 77.5 | 75.8 | | 78.2 |
| F16 | 80.3 | 77.8 | 76.9 | | 78.4 |
| F17 | 79.8 | 77.5 | 83.7 | | 79.6 |
| F18 | 82.6 | 81.9 | 80.9 | | 80.3 |
| F19 | 81.7 | 81.0 | 79.6 | | 82.4 |
| F20 | 82.2 | 80.4 | 82.6 | | 81.9 |
| F24 | | | | | 79.9 |

To determine the effect of irradiation on the polymers in the formulation, the weight average molecular weight of the polymer was evaluated after various times and storage conditions following irradiation. Tables 9 and 10 show results of representative samples. As can be seen from Tables 9 and 10, irradiation does not significantly affect the degradation rate of polymers in tested formulations, but storage at increased temperatures resulted in degradation of polymers in some cases.

TABLE 9

| | MW (Da) | | |
|---|---|---|---|
| Form. | T = 0 | T = 0 | 2 wk 37° C. |
| No. | (nonirradiated) | (irradiated) | (nonirradiated) |
| F4 | 8302 | 7898 | 5808 |
| F5 | 8480 | 8201 | 5393 |
| F6 | 8893 | 8608 | 8189 |
| F7 | 8902 | 8821 | 8497 |
| F8 | 9093 | 9786 | 8877 |
| F9 | 15293 | 14409 | 13524 |
| F10 | 8058 | 7760 | 6962 |
| F11 | 16849 | 15597 | 8439 |
| F12 | 17952 | 16252 | 14606 |
| F13 | 8000 | 7959 | 7634 |
| F14 | 17914 | 17006 | 15242 |
| F15 | 18041 | 17086 | 14257 |
| F16 | 17666 | 17240 | 15920 |
| F17 | 8909 | 8660 | 8556 |
| F18 | 10863 | 10649 | 9989 |
| F19 | 11070 | 11067 | 10276 |
| F20 | 18054 | 17698 | 17408 |

TABLE 10

| Form. | MW (% of T = 0) | | | |
|---|---|---|---|---|
| No. | Irradiated | 2 wk 37° C. | 4 wk 37° C. | 4 wk 25° C. |
| F4 | 95.1 | 70.0 | 53.8 | 79.5 |
| F5 | 96.7 | 63.6 | 43.6 | 69.4 |
| F6 | 96.8 | 92.1 | 82.2 | 96.1 |
| F7 | 99.1 | 95.5 | 88.7 | 97.5 |
| F8 | 107.6 | 97.6 | 88.9 | 100.4 |
| F9 | 94.2 | 88.4 | 76.0 | 93.7 |
| F10 | 96.3 | 86.4 | 72.4 | 91.9 |
| F11 | 92.6 | 50.1 | 35.9 | 66.3 |
| F12 | 90.5 | 81.4 | 67.0 | 90.8 |
| F13 | 99.5 | 95.4 | 89.9 | 97.0 |
| F14 | 94.9 | 85.1 | 71.8 | 91.7 |
| F15 | 94.7 | 79.0 | 64.2 | 88.6 |
| F16 | 97.6 | 90.1 | 78.3 | 93.5 |
| F17 | 97.2 | 96.0 | 90.4 | 100.3 |
| F18 | 98.0 | 92.0 | 81.2 | 97.0 |
| F19 | 100.0 | 92.8 | 82.6 | 98.7 |
| F20 | 98.0 | 96.4 | 90.8 | 99.2 |
| F24 | 94.4 | | | |

### Example 7. Injection Testing of Provided Compositions

The injection characteristics of certain formulations were investigated. Several formulations were tested for injection time using a 1 mL Exel syringe under 5 lbf, delivering 0.5 mL (nominally) volume and using 19 G 1″ (TW) needles. Two syringes were tested per formulation. The results are summarized in Table 11.

TABLE 11

| Form. | Injection |
|---|---|
| No. | Time (sec) |
| F62 | 5.0, 4.8 |
| F73 | 7.6, 7.7 |
| F78 | 11.5, 12.3 |
| F79 | 10.5, 11.0 |

The injection characteristics of formulation F58 were also investigated. Using an Instron materials tester and 3 mL BD disposable syringes with Terumo 19 G×⅝″ needles, two injection tests were performed on each of five vials of formulation for a total of five readings at each of two conditions: (1) to deliver 2.0 mL in 10 seconds using an injection speed of 3.5 mm/s; and (2) to deliver 0.2 mL in 5 seconds using an injection speed of 0.7 mm/s. After each injection test, the amount delivered was measured and recorded. The force during injection was reported from 40% to 80% of the distance traveled at the speed specified in the protocol.

Table 12 presents the results of injection test (1) with F58. The average glide force for the 2.0 mL injections was 43.70 Newtons. Table 13 presents the results of injection test (2) with F58. The average glide force for the 0.2 mL injections was 11.98 Newtons.

TABLE 12

| | Room | Glide | Volume | Time |
|---|---|---|---|---|
| | Temp. | Force | Delivered | Required |
| Run | (° C.) | (N) | (mL) | (s) |
| 1 | 24.2 | 45.29 | 2.07 | 10.3 |
| 2 | 24.4 | 42.95 | 2.05 | 10.3 |
| 3 | 24.3 | 41.72 | 2.05 | 10.3 |
| 4 | 23.6 | 43.73 | 2.03 | 10.2 |
| 5 | 23.9 | 44.80 | 2.01 | 10.1 |
| Average | 24.1 | 43.70 | 2.04 | 10.2 |

TABLE 13

| | Room | Glide | Volume | Time |
|---|---|---|---|---|
| | Temp. | Force | Delivered | Required |
| Run | (° C.) | (N) | (mL) | (s) |
| 1 | 24.0 | 11.90 | 0.21 | 5.3 |
| 2 | 24.1 | 11.01 | 0.21 | 5.2 |
| 3 | 24.3 | 12.79 | 0.20 | 5.1 |
| 4 | 24.0 | 11.33 | 0.21 | 5.4 |
| 5 | 24.1 | 12.86 | 0.21 | 5.2 |
| Average | 24.1 | 11.98 | 0.21 | 5.2 |

The force required at higher speed injection was surprisingly lower than the force required at lower speed injection. Specifically, the higher speed injection was five times faster than the lower speed injection (3.5 mm/s v. 0.7 mm/s). The force required at the higher speed injection (average of 43.70 Newtons) was less than five times the force required at the lower speed injection (average 11.98 Newtons).

### Example 8. In Vitro Release of Provided Compositions

The present disclosure provides formulations that allow for prolonged release when administered (e.g., via injection). This Example provides representative embodiments of formulations that achieve prolonged release.

Representative samples were prepared as generally described in Example 4 above with components as summarized in Table 14. The release of active agent from the samples was measured in aqueous buffer (Dulbecco's PBS, pH 7.4 or 20 mM KH₂PO₄, pH 6.0, 0.9% NaCl) at 37° C. Briefly, 0.5 mL of sample formulation (room temperature) was placed in a fixed surface area cup, which was moved to 100 mL fresh 37° C. buffer at each time point; gentle agitation was performed during release rate testing.

TABLE 14

TAF Delivered 0-24 hrs (%)

| Form. | | St dev |
|---|---|---|
| No. | Average | (n = 3) |
| F17 | 4.8 | 0.1 |
| F5 | 11.1 | 6.4 |
| F20 | 18.9 | 0.7 |
| F4 | 19.0 | 11.5 |
| F24 | 19.5 | 11.3 |
| F10 | 25.4 | 7.2 |
| F19 | 26.5 | 13.3 |
| F26 | 28.4 | 5.9 |
| F23 | 35.5 | 1.3 |
| F18 | 36.6 | 3.7 |
| F6 | 37.4 | 3.4 |
| F25 | 40.8 | 13.2 |
| F27 | 41.4 | 0.9 |
| F12 | 42.8 | 5.8 |
| F9 | 48.9 | 5.2 |
| F7 | 49.6 | 4.8 |
| F13 | 51.7 | 3.9 |
| F8 | 69.3 | 9.0 |
| F14 | 73.9 | 2.4 |
| F15 | 75.0 | 3.9 |
| F16 | 81.1 | 15.4 |
| F11 | 85.8 | 2.5 |

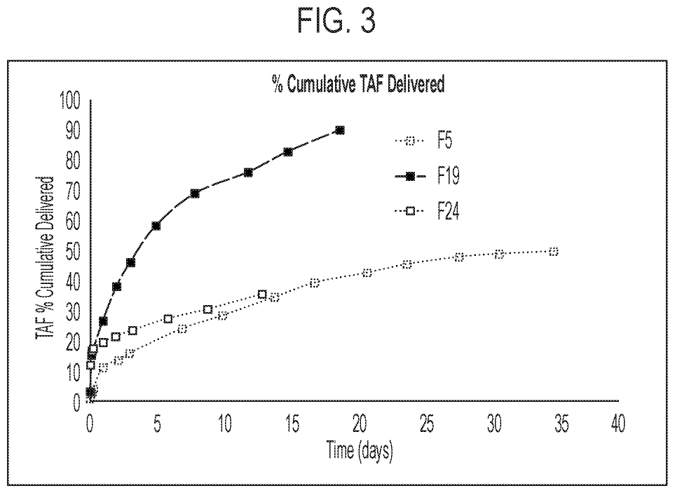

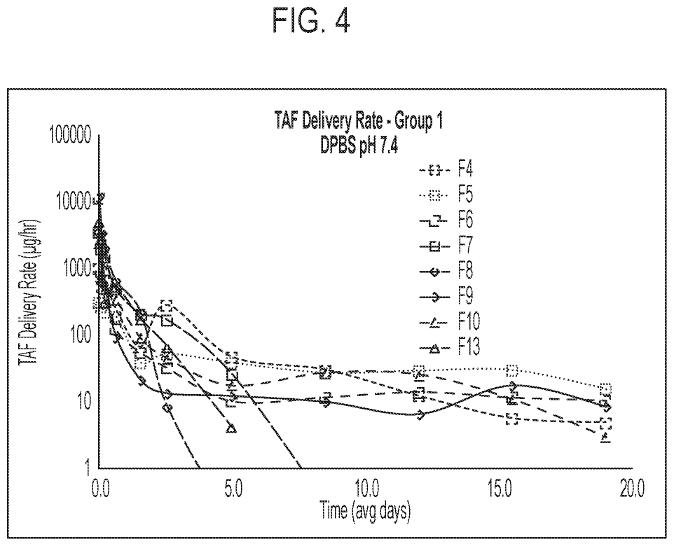

The cumulative release (%) of TAF from selected formulations of Table 14 is plotted in FIG. 3. For formulation F5 in FIG. 3, Dulbecco's PBS pH 7.4 buffer was used for the first 10 days. For all other time points in FIG. 3, 20 mM KH₂PO₄, pH 6.0, with 0.9% NaCl buffer was used. FIG. 4 depicts the delivery rate (μg/h) of TAF of selected formulations of Table 14. For formulations F4, F5, F6, F7 and F10 in FIG. 4, Dulbecco's PBS pH 7.4 buffer was used for the first 10 days. For all other time points in FIG. 4, 20 mM KH₂PO₄, pH 6.0, with 0.9% NaCl buffer was used.

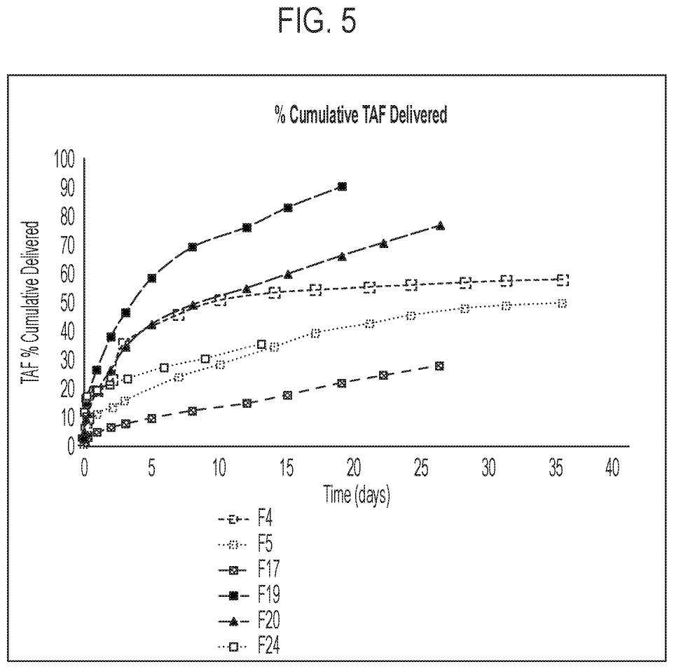

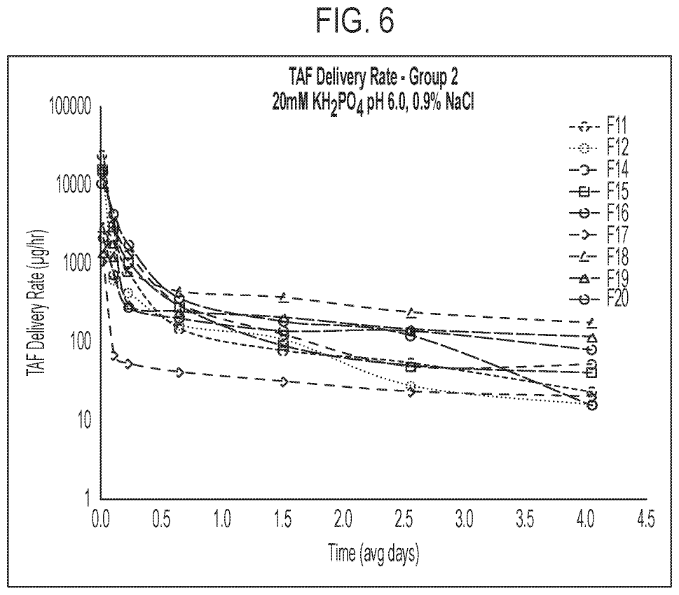

The cumulative release (%) of TAF from selected formulations of Table 14 is plotted in FIG. 5. For formulations F4 and F5 in FIG. 5, Dulbecco's PBS pH 7.4 buffer was used for the first 10 days. For all other time points in FIG. 5, 20 mM KH₂PO₄, pH 6.0, with 0.9% NaCl buffer was used. FIG. 6 depicts the delivery rate (μg/h) of TAF of selected formulations of Table 14 in 20 mM KH₂PO₄, pH 6.0, with 0.9% NaCl buffer.

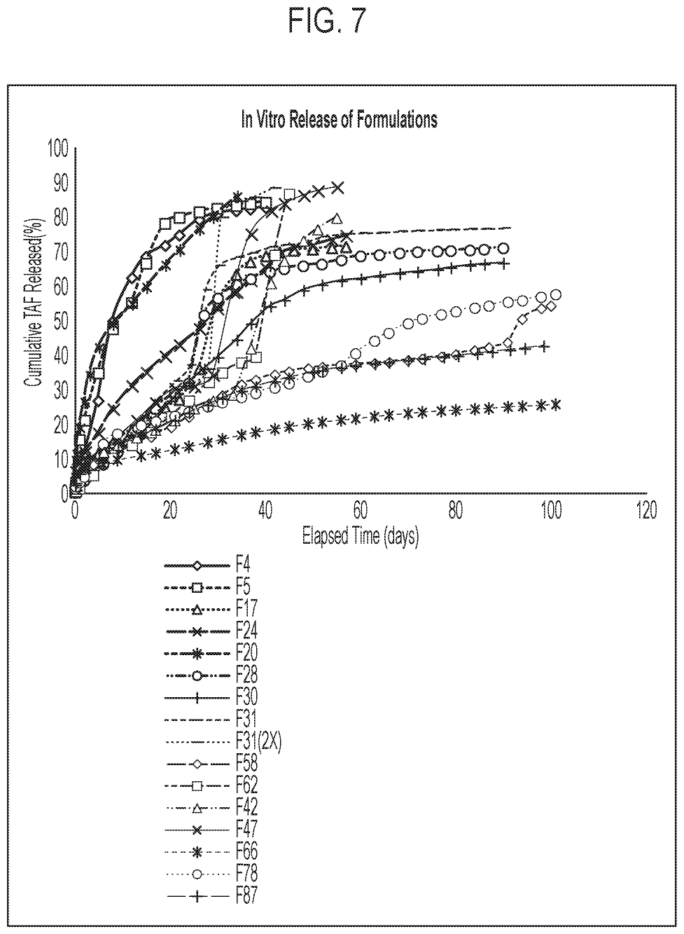

The cumulative release (%) of TAF from additional selected formulations of Table 14 is depicted in FIG. 7. For formulations F4 and F5 in FIG. 7, Dulbecco's PBS pH 7.4 buffer was used for the first 10 days. For all other time points in FIG. 7, 20 mM KH₂PO₄, pH 6.0, with 0.9% NaCl buffer was used.

### Example 9. In Vitro Release of Provided Compositions

The present disclosure provides formulations that allow for prolonged release when administered (e.g., via injection) and/or those that allow for desirable initial release profiles (e.g., to avoid an initial burst release of active agent). This Example provides representative embodiments of formulations that achieve prolonged release and/or desirable initial release profiles.

Representative samples were prepared as generally described in Example 4 above. The release of active agent from the samples was measured in aqueous buffer (Dulbecco's PBS, pH 7.4) at 37° C. Briefly, 0.5 mL of sample formulation (room temperature) was placed in a fixed surface area cup, which was moved to 100 mL fresh 37° C. buffer at each time point; gentle agitation was performed during release rate testing.

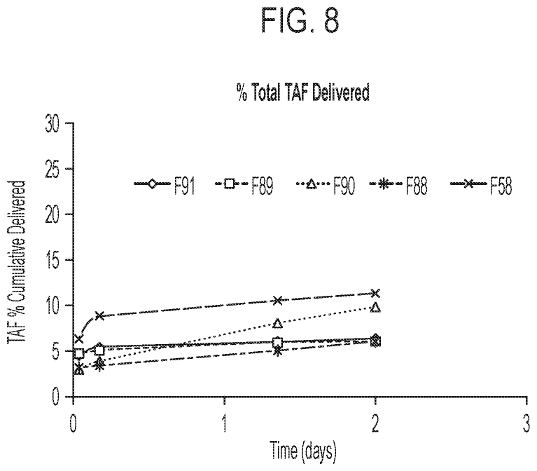

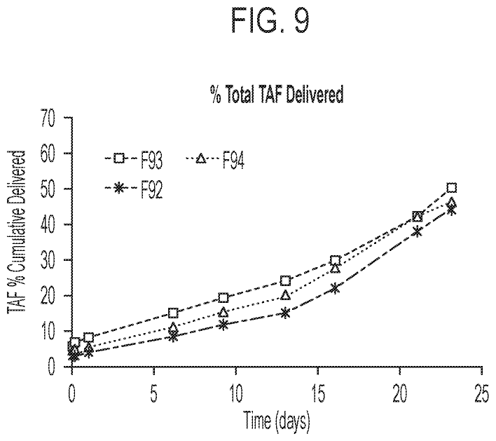

FIG. 8 shows cumulative release (%) of TAF from selected formulations of Table 4B over a 2-day period. FIG. 9 shows cumulative release (%) of TAF from selected formulations over a 24-day period.

### Example 10. Pharmacokinetics of Provided Compositions in Dogs

Provided compositions were administered via single subcutaneous (SC) injection to male beagle dogs (e.g., 0.5 mL provided formulation containing a dose of 45 mg TAF free base equivalent). Plasma and peripheral blood mononuclear cell (PBMC) samples were collected at certain time points postdose. Plasma concentrations of TAF and PBMC concentrations of TFV-DP were determined via LC-MS/MS, and the number of days was recorded for which a detectable level of TAF or TFV-DP was observed.

Table 15 summarizes the results of the PK evaluation in dogs.

TABLE 15

| | Detectable | TAF | Detectable | TFV-DP |
|---|---|---|---|---|
| | TAF plasma | plasma | TFV-DP | intracellular |
| Form. | level | LLOQ | intracellular | LLOQ |
| No. | (days)^{a} | (ng/mL)^{b} | level (days)^{c} | (nM)^{d} |
| F1 | 1.5 | 0.5 | ND | 10 |
| F2 | 3.3 | 0.5 | 6 | 10 |
| F3 | 2.2 | 0.5 | ND | 10 |
| F4 | 4.70 | 0.1 | ND | 10 |
| F5 | 4.0 | 0.1 | ND | 10 |
| F17 | 77.7 | 0.01 | 85.0 | 10 |
| F20 | 3.30 | 0.1 | 37.0 | 10 |
| F24 | 35.0 | 0.01 | ND | 10 |
| F28 | 34.0 | 0.01 | ND | 10 |
| F30 | 49.3 | 0.01 | 49.7 | 10 |
| F31 | 41.3 | 0.01 | 80.0 | 10 |
| F42 | 20.6 | 0.01 | ND | 10 |
| F47 | 26.0 | 0.01 | ND | 10 |
| F58 | 33.0 | 0.01 | 54.3 | 10 |
| F59 | 29.0 | 0.01 | 49.0 | 10 |
| F62 | 39.3 | 0.01 | 54.3 | 10 |
| F66 | 41.3 | 0.01 | 27 | 10 |
| F78 | 45.0 | 0.01 | 27 | 10 |
| F87 | 28.3 | 0.01 | ND | 10 |

^{a}Time of last detected TAF plasma concentration; all values are average of 3 dogs.
^{b}Lower limit of quantification for TAF plasma concentration determination by LC-MS/MS assay.
^{c}Time of last detected intracellular TFV-DP concentration in PBMCs; all values are average of 3 dogs.
^{d}Lower limit of quantification for intracellular TFV-DP concentration in PBMCs determined by LC-MS/MS assay. LLOQ of cell counting assay was 2.0 million cells/sample.

### Example 11. Effect of Polymer Molecular Weight on Pharmacokinetics of Provided Compositions in Dogs

Provided compositions were administered via single subcutaneous (SC) injection to male beagle dogs and were evaluated as described in Example 9. Each formulation tested in this Example comprised 7.8 wt % tenofovir alafenamide sebacate and a vehicle comprising SAIB/PC/PLGA 90-10, 62/28/10). The PLGA 90-10 had varying MWs.

TABLE 16

| | PLGA | Detectable | TAF | Detectable | TFV-DP |
|---|---|---|---|---|---|
| | 90-10 | TAF plasma | plasma | TFV-DP | intracellular |
| Form. | MW | level | LLOQ | intracellular | LLOQ |
| No. | (kDa)^{a} | (days)^{b} | (ng/mL)^{c} | level (days)^{d} | (nM)^{e} |
| F58 | 18 | 33.0 | 0.01 | 54.3 | 10 |
| F59 | 18 | 29.0 | 0.01 | 49.0 | 10 |
| F95 | 13 | 23.0 | 0.01 | 42.0 | 10 |
| F96 | 16 | 15.6 | 0.01 | 30.0 | 10 |
| F97 | 20 | 15.6 | 0.01 | 36.0 | 10 |

^{a}Measured molecular weight.
^{b}Time of last detected TAF plasma concentration; all values are average of 3 dogs.
^{c}Lower limit of quantification for TAF plasma concentration determination by LC-MS/MS assay.
^{d}Time of last detected intracellular TFV-DP concentration in PBMCs; all values are average of 3 dogs.
^{e}Lower limit of quantification for intracellular TFV-DP concentration in PBMCs determined by LC-MS/MS assay. LLOQ of cell counting assay was 2.0 million cells/sample.

### INCORPORATION BY REFERENCE

All publications, patents, and patent applications mentioned herein are hereby incorporated by reference in their entirety.

### EQUIVALENTS

Those skilled in the art will recognize, or be able to ascertain, using no more than routine experimentation, numerous equivalents to the specific embodiments described specifically herein. Such equivalents are intended to be encompassed in the scope of the following claims.

1. A composition comprising:
tenofovir alafenamide, or a pharmaceutically acceptable salt thereof; and
(ii) sucrose acetate isobutyrate.

2. The composition of claim 1, wherein the composition comprises from about 25 wt % to about 65 wt % sucrose acetate isobutryate, based on the total weight of the composition.

3. The composition of claim 1 or 2, wherein the composition comprises from about 40 wt % to about 60 wt % sucrose acetate isobutyrate, based on the total weight of the composition.

4. The composition of any one of claims 1-3, wherein the composition further comprises poly(lactic acid)(glycolic acid).

5. The composition of claim 4, wherein the poly(lactic acid)(glycolic acid) comprises lactic acid repeat units and glycolic acid repeat units in a molar ratio from about 70:30 to about 100:0, respectively.

6. The composition of claim 4, wherein the poly(lactic acid)(glycolic acid) comprises lactic acid repeat units and glycolic acid repeat units in a molar ratio from about 65:35 to about 95:5, respectively.

7. The composition of any one of claims 4-6, wherein the poly(lactic acid)(glycolic acid) has a weight average molecular weight from about 5 kDa to about 25 kDa when measured using gel permeation chromatography.

8. The composition of any one of claims 4-6, wherein the poly(lactic acid)(glycolic acid) has a weight average molecular weight from about 15 kDa to about 55 kDa when measured using gel permeation chromatography.

9. The composition of any one of claims 4-8, wherein the composition comprises from about 5 wt % to about 30 wt % poly(lactic acid)(glycolic acid), based on the total weight of the composition.

10. The composition of any one of claims 4-9, wherein the composition comprises from about 5 wt % to about 20 wt % poly(lactic acid)(glycolic acid), based on the total weight of the composition.

11. The composition of any one of the preceding claims, wherein the composition further comprises propylene carbonate.

12. The composition of any one of the preceding claims, wherein the composition comprises from about 10 wt % to about 40 wt % propylene carbonate, based on the total weight of the composition.

13. The composition of any one of claims 1-11, wherein the composition comprises from about 20 wt % to about 60 wt % propylene carbonate, based on the total weight of the composition.

14. The composition of any one of the preceding claims, wherein the composition comprises from about 5 wt % to about 30 wt % tenofovir alafenamide, or a pharmaceutically acceptable salt thereof, based on the total weight of the composition.

15. The composition of claim 14, wherein the tenofovir alafenamide, or a pharmaceutically acceptable salt thereof, is tenofovir alafenamide sebacate.

16. The composition of any one of the preceding claims, wherein the composition comprises, based on the total weight of the composition:
(i) from about 5 wt % to about 15 wt % tenofovir alafenamide sebacate;
(ii) from about 5 wt % to about 10 wt % poly(lactic acid)(glycolic acid);
(iii) from about 20 wt % to about 30 wt % propylene carbonate; and
(iv) from about 50 wt % to about 60 wt % sucrose acetate isobutyrate.

17. The composition of any one of the preceding claims, wherein the composition has been stored for at least 1 week.

18. The composition of any one of the preceding claims, wherein the composition has been sterilized, optionally wherein the composition has been sterilized by gamma irradiation.

19. The composition of any one of the preceding claims, wherein the composition achieves one or more of the following characteristics in a subject when administered subcutaneously to the subject as a single dose:
(1) plasma tenofovir alafenamide concentration greater than 0.01 ng/mL for at least 10 days; and
(2) intracellular tenofovir diphosphate concentration in peripheral blood mononuclear cells greater than 10 nM for at least 10 days.

20. A method of manufacturing the composition of any one of the preceding claims, comprising:
(a) providing tenofovir alafenamide, or a pharmaceutically acceptable salt thereof; and
(b) combining the tenofovir alafenamide, or a pharmaceutically acceptable salt thereof, with sucrose acetate isobutyrate, poly(lactic acid)(glycolic acid), and propylene carbonate to form the composition.

21. A method of administering a therapeutically effective amount of tenofovir alafenamide, comprising administering to a subject the composition of any one of claims 1-19.

22. A method of treating or preventing HIV infection, comprising administering the composition of any one of claims 1-19 to subject in need thereof.

23. A method of treating or preventing HBV infection, comprising administering the composition of any one of claims 1-19 to subject in need thereof.

24. The method of any one of claims 21-23, wherein the subject is receiving or has received an additional therapeutic agent.
